(19) United States
(12) Patent Application Publication
(10) Pub. No.: US 2025/0082774 A1
(43) Pub. Date: Mar. 13, 2025

(54) PEPTIDE LINKERS COMPRISING TWO OR MORE PAYLOADS

(71) Applicant: ARARIS BIOTECH AG, Au (CH)

(72) Inventors: Romain BERTRAND, Au (CH); Isabella ATTINGER-TOLLER, Au (CH); Rachael FAY, Au (CH); Dragan GRABULOVSKI, Au (CH); Philipp SPYCHER, Au (CH)

(21) Appl. No.: 18/803,344

(22) Filed: Aug. 13, 2024

Related U.S. Application Data
(63) Continuation of application No. PCT/EP2023/054455, filed on Feb. 22, 2023.

(30) Foreign Application Priority Data
Feb. 22, 2022 (EP) 22158100.2

(51) Int. Cl. A61K 47/68; A61P 35/00; C07K 16/28; C07K 16/32

(52) U.S. Cl. CPC A61K 47/6851; A61K 47/68031; A61K 47/68037; A61P 35/00; C07K 16/2803; C07K 16/32; C07K 2317/24

(57) ABSTRACT

The present invention relates to a peptide linker comprising (a) an amino acid residue comprising a primary amine; and (b) two or more payloads; wherein each of the two or more payloads can be independently attached to: (i) an N-terminal end of the peptide linker, (ii) a C-terminal end of the peptide linker, or (iii) a side chain of an amino acid residue comprised in the peptide linker. Further, the present invention relates to antibody-payload conjugates comprising the peptide linker of the invention, methods for generating said antibody-payload conjugates and uses thereof.

### RELATED APPLICATIONS

This application is a continuation of International Patent Application No. PCT/EP2023/054455, filed Feb. 22, 2023, which claims priority to European Patent Application No. 22158100.2, filed Feb. 22, 2022, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Nov. 19, 2024, is named 756618_ARA9-007PCCON_ST26.xml and is 107,532 bytes in size.

### BACKGROUND OF THE INVENTION

The present invention relates to methods for generating an antibody-payload conjugate by means of a transglutaminase. The invention further provides peptide linkers comprising two or more payloads for the generation of antibody-payload conjugates. Further encompassed are pharmaceutical compositions comprising the antibody-payload conjugates of the invention and uses thereof.

Antibody-based therapeutics have played an important role in targeted therapy for various disorders, such as cancers and immunological diseases. In recent years, antibody drug conjugates (ADCs) have been explored extensively for effective delivery of drugs to target sites. While many ADCs have shown impressive anti-cancer activity, many patients do not respond to these treatments, experience severe side-effects before signs of efficacy or experience a relapse after a certain period of time, so there is still a large medical need for novel ADC formats which have favorable drug-like properties, can be produced in sufficient quantity and quality at reasonable costs to support drug development, and which are suitable as therapeutics.

A key step in the preparation of an ADC is the covalent conjugation step of a payload to the antibody.

Most ADCs in current clinical development were made by conjugation to endogenous lysine or cysteine residues of the antibody, carefully controlling the average degree of modification to yield an average drug-to-antibody ratio (DAR) in the range of 3.5-4.0. More recently, ADCs with a DAR7-8 showed significantly improved efficacy because of the delivery of much more toxic payloads to the tumor site (Ogitani et al., 2016. Clin Cancer Res, 22(20): 5097-5108).

Enzymatic conjugation has shown great interest since these conjugation reactions are typically fast, site-specific and can be done under physiological conditions. Among the available enzymes, microbial transglutaminase (MTG) from the species Streptomyces mobaraensis has found increasing interest as an attractive alternative to conventional chemical protein conjugation of functional moieties including antibodies. The MTG catalyzes under physiological conditions a transamidation reaction between a ‘reactive’ glutamine of a protein or peptide and a ‘reactive’ lysine residue of a protein or peptide, whereas the latter can also be a simple, low molecular weight primary amine such as a 5-aminopentyl group (Jeger S. et al., 2010, Angew. Chem. Int. Ed., 49, 9995-9997). Therefore, transglutaminases (TGase) transfer a moiety having an amine donor group to an acceptor glutamine residue through transglutamination.

Full-length IgG antibodies of human isotype contain a conserved glutamine residue at position 295 of the heavy chain (Q295). Because this glutamine 295 residue is in close proximity to an N-glycosylation site (N297), it was generally believed that Q295 on the full-length antibody is inaccessible to TGase when the antibody is N-glycosylated. To allow TGase acting on full-length antibodies, the Fc region of the antibody was deglycosylated or mutated to remove the N-glycosylation site prior to the TGase-mediated conjugation. For example, Jeger et al. described that conjugation of an antibody using transglutaminase as an enzyme happens at the Q295 residue, however, conjugation was only possible upon removal of the glycan moiety at the asparagine residue 297 (N297) with PNGase F, while glycosylated antibodies could not be conjugated efficiently (conjugation efficiency below 20%) (Jeger S. et al., 2010, Angew. Chem. Int. Ed., 49, 9995-9997; Mindt T. et al. 2008, Bioconj Chem, 9, 271-278).

Alternatively, glutamine-containing sequence “tags” have been inserted into the antibodies' light or heavy chains to provide acceptor glutamine sites (see for example WO 2012/059882). Hence, historically, site-specific ADC technologies relied on engineered antibody mutants, which may result in potential immunogenicity and in vivo instability.

Hu and Allen discovered that conjugation at Q295 of a native, glycosylated antibody can be achieved with an engineered transglutaminase (WO 2015/191883). Indeed, the authors showed higher conjugation efficiencies using engineered transglutaminases compared to wild-type transglutaminases.

More recently, Spycher et al. disclosed a wild-type transglutaminase-based conjugation approach which does not require prior deglycosylation of the antibody for payload conjugation (Spycher et al., WO 2019/057772 and WO 2020/188061). Surprisingly, Spycher et al. could show high conjugation efficiencies with lysine- or glycine-based linkers.

Schematically, Hu and Allen (WO 2015/191883), as well as Spycher et al. (WO 2019/057772 and WO 2020/188061), described two-step and one-step conjugation approaches. However, neither Hu and Allen nor Spycher et al. experimentally demonstrated the conjugation of linkers comprising two or more payloads to native glycosylated antibodies in a single step. In contrast, it was postulated that a two-step process, in which a linker comprising two functional groups is conjugated to an antibody in a first step and the payloads are then chemically coupled to the antibody-linker conjugate in a second step, is required for obtaining DAR4 ADCs. In particular, it was assumed that direct conjugation of linkers comprising two or more payloads to antibodies in a single step will be inefficient due to steric hindrance at the binding pocket of the transglutaminase. Another concern was that linkers comprising two or more payloads have been reported to have low solubility and are prone to aggregation.

More recently, a research group reported the preparation of DAR4 ADCs with the use of a wild-type transglutaminase (Yamazaki et al. 2021, Nat Comm). The synthesis of the ADCs required both antibody re-engineering (mutation of N297 to an alanine and therefore remove the glycan moiety at the asparagine residue 297) and a two-step chemo-enzymatic approach, further indicating that it was difficult to obtain DAR4 ADCs in a straightforward one-step process.

From a manufacturing viewpoint, a one-step process is clearly preferred. Unfortunately, efficient conjugation of native glycosylated antibodies with linkers comprising two or more payloads has not been achieved so far. Consequently, there is a need in the art for linkers with two or more payloads that can be efficiently conjugated tow native glycosylated antibodies.

Thus, the objective technical problem of the present invention can be formulated as the provision of linkers comprising two or more payloads for the efficient conjugation to native glycosylated antibodies.

### SUMMARY OF THE INVENTION

The present invention is characterized in the herein provided embodiments and claims. In particular, the present invention relates, inter alia, to the following embodiments:

1. A peptide linker comprising
a) an amino acid residue comprising a primary amine; and
b) two or more payloads;
wherein each of the two or more payloads can be independently attached to:
i) an N-terminal end of the peptide linker,
ii) a C-terminal end of the peptide linker, or
iii) a side chain of an amino acid residue comprised in the peptide linker.

2. The peptide linker according to embodiment 1, wherein the primary amine comprised in the amino acid residue is
a) a primary amine in a side chain of a lysine, a lysine derivative or a lysine mimetic; or
b) a primary amine comprised in an N-terminal amino acid residue having the structure NH₂—(Y)—COOH.

3. The peptide linker according to embodiment 2, wherein Y is (R₂C)ₙ and wherein n is an integer ranging from 1 to 20, from 1 to 15, from 1 to 10.

4. The peptide linker according to embodiment 3, wherein at least one R moiety of each —(R₂C)— monomer is hydrogen or wherein both R moieties of each —(R₂C)— monomer are hydrogen.

5. The peptide linker according to any one of embodiments 1 to 4, wherein the linker comprises not more than 25, 20, 15, 14, 13, 12, 11, 10, 9, 8, 7, 6, 5, 4 amino acid residues.

6. The peptide linker according to any one of embodiments 1 to 5, wherein the linker comprises at least one arginine and/or histidine residue.

7. The peptide linker according to any one of embodiments 1 to 6, wherein the linker comprises the sequence motif RK.

8. The peptide linker according to any one of embodiments 1 to 7, wherein the linker comprises any one of the amino acid sequences set forth in SEQ ID NO:1-29.

9. The peptide linker according to any one of embodiments 1 to 8, wherein the linker comprises between 2 and 4 payloads.

10. The peptide linker according to any one of embodiments 1 to 9, wherein at least one of the two or more payloads is attached to the peptide linker via a chemical linker.

11. The peptide linker according to embodiment 10, wherein the chemical linker is an enzymatically and/or chemically cleavable linker.

12. The peptide linker according to embodiment 10 or 11, wherein the chemical linker is or comprises a self-immolative linker.

13. The peptide linker according to embodiment 12, wherein the self-immolative linker comprises
a) a p-aminobenzyl alcohol moiety; or
b) a 2,4-bis(hydroxymethyl)aniline moiety; or
c) a p-aminobenzyl quaternary ammonium; or
d) a ethylenediamine-based moiety; or
e) an (aminomethyl)pyrrolidine-based moiety; or
f) aminomethyl-based moiety.

14. The peptide linker according to embodiment 13, wherein the hydroxyl group comprised in the p-aminobenzyl alcohol moiety forms a carbamate with a payload.

15. The peptide linker according to embodiment 13, wherein each of the hydroxyl groups comprised in the 2,4-bis(hydroxymethyl)aniline moiety forms a carbamate with a payload.

16. The peptide linker according to embodiment 13, wherein the quaternary ammonium cation comprised in the p-aminobenzyl quaternary ammonium originates from an amine comprised in the payload.

17. The peptide linker according to embodiment 13, wherein the amino group comprised in the ethylenediamine-based moiety, or in the (aminomethyl)pyrrolidine-based moiety, forms a carbamate with a payload.

18. The peptide linker according to embodiment 13, wherein the amino group comprised in the aminomethyl-based moiety forms an hemiaminal, or a thiohemiaminal, with a payload.

19. The peptide linker according to any one of embodiments 1 to 18, wherein at least one payload is attached to a side chain of an amino acid residue comprised in the peptide linker.

20. The peptide linker according to embodiment 19, wherein at least one payload is attached to a side chain of a glutamate, aspartate, tryptophan, cysteine, lysine, tyrosine, serine or threonine residue, or their respective derivatives or mimetics.

21. The peptide linker according to any one of embodiments 1 to 20, wherein the peptide linker comprises two peptide moieties, and wherein the two peptide moieties are connected via their N-terminal amino acid residues with a dicarboxylic acid linker, or an activated version thereof.

22. The peptide linker according to any one of embodiments 1 to 21, wherein the payload is at least one of:
a toxin;
a cytokine;
a growth factor;
a radionuclide;
a hormone;
an anti-viral agent;
an anti-bacterial agent;
a fluorescent dye;
an immunoregulatory/immunostimulatory agent;
a half-life increasing moiety;
a solubility increasing moiety;
a polymer-toxin conjugate;
a nucleic acid;
a biotin or streptavidin moiety;
a vitamin;
a protein degradation agent (‘PROTAC’);
a ligand or substrate of a receptor;
a target binding moiety; and/or
an anti-inflammatory agent.

23. The peptide linker according to embodiments 22, wherein the toxin is at least one selected from the group consisting of:
a pyrrolobenzodiazepine (e.g., PBD);
an auristatin (e.g., MMAE, MMAF);
a maytansinoid (e.g., maytansine, DM1, DM4, DM21);
a duocarmycin;
a nicotinamide phosphoribosyltransferase (NAMPT) inhibitor;
a tubulysin;
an enediyne (e.g., calicheamicin);
an anthracycline derivative (PNU) (e.g., doxorubicin);
a pyrrole-based kinesin spindle protein (KSP) inhibitor;
a cryptophycin;
a drug efflux pump inhibitor;
a sandramycin;
a thymidylate synthase inhibitor;
an amanitin (e.g., a-amanitin); and
a camptothecin (e.g., exatecans, deruxtecans).

24. The peptide linker according to any one of embodiments 1 to 23, wherein the two or more payloads are identical.

25. The peptide linker according to any one of embodiments 1 to 23, wherein at least two of the two or more payloads differ from each other.

26. The peptide linker according to any one of embodiments 1 to 25, wherein the linker is suitable to serve as substrate for a transglutaminase.

27. An antibody-payload conjugate comprising an antibody conjugated to the peptide linker according to any one of embodiments 1 to 26.

28. The antibody-payload conjugate according to embodiment 27, wherein the peptide linker is conjugated to the antibody via an isopeptide bond formed between a γ-carboxamide group of a glutamine residue comprised in the antibody and the primary amine comprised in an amino acid residue of the peptide linker.

29. The antibody-payload conjugate according to embodiment 27 or 28, wherein the antibody is an IgG antibody.

30. The antibody-payload conjugate according to embodiment 29, wherein the peptide linker is conjugated to a glutamine residue comprised in an Fc domain of the antibody.

31. The antibody-payload conjugate according to embodiment 30, wherein the glutamine residue to which the peptide linker is conjugated is glutamine residue Q295 (EU numbering) of the C_{H}2 domain of an IgG antibody.

32. The antibody-payload conjugate according to embodiment 29, wherein the glutamine residue to which the peptide linker is conjugated has been introduced into the heavy or light chain of the antibody by molecular engineering.

33. The antibody-payload conjugate according to embodiment 32, wherein the glutamine residue that has been introduced into the heavy or light chain of the antibody by molecular engineering is N297Q (EU numbering) of the C_{H}2 domain of an aglycosylated IgG antibody.

34. The antibody-payload conjugate according to embodiment 32, wherein the glutamine residue that has been introduced into the heavy or light chain of the antibody by molecular engineering is comprised in a peptide that has been (a) integrated into the heavy or light chain of the antibody or (b) fused to the N- or C-terminal end of the heavy or light chain of the antibody.

35. The antibody-payload conjugate according to embodiment 34, wherein the peptide comprising the Gln residue has been fused to the C-terminal end of the heavy chain of the antibody.

36. The antibody-payload conjugate according to any one of embodiments 29 to 32 or 34 to 35, wherein the IgG antibody is a glycosylated IgG antibody.

37. The antibody-payload conjugate according to embodiment 36, wherein the IgG antibody is glycosylated at residue N297 (EU numbering) of the C_{H}2 domain.

38. The antibody-payload conjugate according to any one of embodiments 27 to 37, wherein the antibody is selected from the group consisting of: Brentuximab, Trastuzumab, Gemtuzumab, Inotuzumab, Avelumab, Cetuximab, Rituximab, Daratumumab, Pertuzumab, Vedolizumab, Ocrelizumab, Tocilizumab, Ustekinumab, Golimumab, Obinutuzumab, Sacituzumab, Belantamab, Polatuzumab, Enfortumab, Endrecolomab, Gemtuzumab, Loncastuximab, Mecbotamab, Adecatumumab, D93, Gatipotuzumab, Labetuzumab, Tusamitamab, Upifitamab, Lifastuzumab, Mirvetuximab, Sofituzumab, Anetumab, Tisotumab, Cofituzumab, Praluzatamab, Ladriatuzumab, Belantamab, Patritumab, Cetuximab, Nimotuzumab, Matuzumab, Portuzumab, Citatuzumab, Tucotuzumab and Endrecolomab.

39. The antibody-payload conjugate according to any one of embodiments 27 to 38, wherein the antibody is selected from the group consisting of: Brentuximab, Gemtuzumab, Trastuzumab, Inotuzumab, Polatuzumab, Enfortumab, Sacituzumab and Belantamab.

40. The antibody-payload conjugate according to any one of embodiments 27 to 39, wherein the antibody is Polatuzumab or Trastuzumab or Enfortumab.

41. A method for the preparation of an antibody-payload conjugate comprising a step of conjugating a peptide linker according to any of embodiments 1 to 26 to an antibody.

42. A method for the conjugation of a peptide linker comprising two or more payloads to an antibody using a transglutaminase (TG), the method comprising a) mixing the antibody, the peptide linker and the TG within a fluid, thereby conjugating the linker-payload to the antibody in one step under the catalyzing effect of the TG, and b) extracting the conjugate obtained in step a) from the fluid.

43. The method according to embodiment 42, wherein the peptide linker is the peptide linker of any one of embodiments 1 to 26.

44. The method according to embodiment 42 or 43, wherein the peptide linker is conjugated to a glutamine residue comprised in the antibody via a primary amine comprised in an amino acid residue of the peptide linker.

45. The method according to any one of embodiments 41 to 44, wherein the antibody is an antibody fragment.

46. The method according to any one of embodiments 41 to 44, wherein the antibody is an IgA, IgD, IgE, IgG or IgM antibody.

47. The method according to any one of embodiments 41 to 46, wherein the peptide linker is conjugated to a glutamine residue comprised in an Fc domain of the antibody.

48. The method according to any one of embodiments 41 to 47, wherein the glutamine residue to which the peptide linker is conjugated is glutamine residue Q295 (EU numbering) of the C_{H}2 domain of an IgG antibody.

49. The method according to any one of embodiments 41 to 47, wherein the glutamine residue to which the peptide linker is conjugated has been introduced into the heavy or light chain of the antibody by molecular engineering.

50. The method according to embodiment 49, wherein the glutamine residue that has been introduced into the heavy or light chain of the antibody by molecular engineering is N297Q (EU numbering) of the C_{H}2 domain of an aglycosylated IgG antibody.

51. The method according to embodiment 50, wherein the glutamine residue that has been introduced into the heavy or light chain of the antibody by molecular engineering is comprised in a peptide that has been (a) integrated into the heavy or light chain of the antibody or (b) fused to the N- or C-terminal end of the heavy or light chain of the antibody.

52. The method according to embodiment 51, wherein the peptide comprising the Gln residue has been fused to the C-terminal end of the heavy chain of the antibody.

53. The method according to any one of embodiments 41 to 49 or 51 to 52, wherein the antibody is a glycosylated IgG antibody.

54. The method according to embodiment 53, wherein the IgG antibody is glycosylated at residue N297 (EU numbering) of the C_{H}2 domain.

55. The method according to any one of embodiments 41 to 54, wherein the antibody is selected from the group consisting of: Brentuximab, Trastuzumab, Gemtuzumab, Inotuzumab, Avelumab, Cetuximab, Rituximab, Daratumumab, Pertuzumab, Vedolizumab, Ocrelizumab, Tocilizumab, Ustekinumab, Golimumab, Obinutuzumab, Sacituzumab, Belantamab, Polatuzumab, Enfortumab, Endrecolomab, Gemtuzumab, Loncastuximab, Mecbotamab, Adecatumumab, D93, Gatipotuzumab, Labetuzumab, Tusamitamab, Upifitamab, Lifastuzumab, Mirvetuximab, Sofituzumab, Anetumab, Tisotumab, Cofituzumab, Praluzatamab, Ladriatuzumab, Belantamab, Patritumab, Cetuximab, Nimotuzumab, Matuzumab, Portuzumab, Citatuzumab, Tucotuzumab and Endrecolomab.

56. The method according to any one of embodiments 41 to 55, wherein the antibody is selected from the group consisting of: Brentuximab, Gemtuzumab, Trastuzumab, Inotuzumab, Polatuzumab, Enfortumab, Sacituzumab and Belantamab.

57. The method according to any one of embodiments 41 to 56, wherein the antibody is Polatuzumab or Trastuzumab or Enfortumab.

58. The method according to any one of embodiments 41 to 57, wherein the peptide linker is conjugated to a γ-carboxamide group of a Gln residue comprised in the antibody.

59. The method according to any one of embodiments 41 to 58, wherein the peptide linker is suitable for conjugation to a glycosylated antibody with a conjugation efficiency of at least 20%, 30%, 40%, 50%, 60%, 70%, 75%, 80%, 85%, 90% or 95%.

60. The method according to any one of embodiments 41 to 59, wherein the transglutaminase is a microbial transglutaminase (MTG).

61. The method according to embodiment 60, wherein the microbial transglutaminase is derived from a Streptomyces species, in particular Streptomyces mobaraensis.

62. The method according to any one of embodiments 41 to 61, wherein the antibody is contacted with 2-100 molar equivalents of linker.

63. The method according to any one of embodiments 41 to 62, wherein the antibody is added to the conjugation reaction at a concentration of 0.1-50 mg/mL.

64. The method according to any one of embodiments 41 to 63, wherein the transglutaminase is added to the conjugation reaction at a concentration of less than 200 U/mg antibody.

65. The method according to any one of embodiments 41 to 64, wherein the conjugation reaction is carried out in a buffered solution.

66. The method according to embodiment 65, wherein the buffered solution comprises
a) a pH ranging from 5 to 10; and/or
b) a buffer concentration ranging from 10 to 1000 mM; and/or
c) a salt concentration ranging below 250 mM.

67. An antibody-payload conjugate which has been produced with the method according to any one of embodiments 41 to 66.

68. A pharmaceutical composition comprising the antibody-payload conjugate according to any one of embodiments 27 to 40 or embodiment 67 and at least one pharmaceutically acceptable ingredient.

69. The pharmaceutical composition according to embodiment 68 comprising at least one additional therapeutically active agent.

70. The antibody-payload conjugate according to any one of embodiments 27 to 40 or embodiment 67, or the pharmaceutical composition according to embodiment 68 or 69 for use in therapy and/or diagnostics.

71. The antibody-payload conjugate according to any one of embodiments 27 to 40 or embodiment 67, or the pharmaceutical composition according to embodiment 68 or 69 for use in the treatment of a patient
suffering from,
being at risk of developing, and/or
being diagnosed for
   a neoplastic disease, a neurological disease, an autoimmune disease, an inflammatory disease or an infectious disease.

72. The antibody-payload conjugate or the pharmaceutical composition for use according to embodiment 71, wherein the antibody-payload conjugate comprises Polatuzumab and wherein the neoplastic disease is a B-cell associated cancer.

73. The antibody-payload conjugate or the pharmaceutical composition for use according to embodiment 72, wherein the B-cell associated cancer is non-Hodgkin lymphoma, in particular wherein the B-cell associated cancer is diffuse large B-cell lymphoma.

74. The antibody-payload conjugate or the pharmaceutical composition for use according to embodiment 72 or 73, wherein the antibody-payload conjugate or the pharmaceutical composition is administered in combination with bendamustine and/or rituximab.

75. The antibody-payload conjugate or the pharmaceutical composition for use according to embodiment 71, wherein the antibody-payload conjugate comprises Trastuzumab and wherein the neoplastic disease is a HER2-positive cancer, in particular HER2-positive breast, gastric, ovarian or lung cancer.

76. The antibody-payload conjugate or the pharmaceutical composition for use according to embodiment 75, wherein the antibody-payload conjugate or the pharmaceutical composition is administered in combination with lapatinib, capecitabine and/or a taxane.

77. The antibody-payload conjugate or the pharmaceutical composition for use according to embodiment 71, wherein the antibody-payload conjugate comprises Enfortumab or an Enfortumab variant and wherein the neoplastic disease is a Nectin-4 positive cancer, in particular Nectin-4 positive pancreatic cancer, lung cancer, bladder cancer or breast cancer.

78. The antibody-payload conjugate or the pharmaceutical composition for use according to embodiment 77, wherein the antibody-payload conjugate or the pharmaceutical composition is administered in combination with a platinum-based chemotherapeutic agent and/or Pembrolizumab.

79. Use of the antibody-payload conjugate according to any one of embodiments 27 to 40 or embodiment 67, or the pharmaceutical composition according to embodiment 68 or 69 for the manufacture of a medicament for the treatment of a patient
suffering from,
being at risk of developing, and/or
being diagnosed for
   a neoplastic disease, neurological disease, an autoimmune disease, an inflammatory disease or an infectious disease.

80. A method of treating or preventing a neoplastic disease, said method comprising administering to a patient in need thereof the antibody-payload conjugate according to any one of embodiments 27 to 40 or embodiment 67, or the pharmaceutical composition according to embodiment 68 or 69.

That is, the present invention is based, at least in part, on the surprising finding that peptide linkers comprising two or more payloads can be efficiently conjugated to native glycosylated antibodies. As can be seen in the appended Examples, peptide linkers comprising two or more payloads can be conjugated to native glycosylated antibodies in a single reaction step with exceptionally high efficiencies of at least 60%. Moreover, peptide linkers comprising two payloads can be conjugated to native glycosylated antibodies in a single reaction step with 80-100% efficiency.

To the surprise of the inventors, it was found out that peptide linkers according to this invention are particularly well-suited for 1-step conjugation of ADCs with a DAR≥4, in contrast to non-peptide linkers that achieved only less than 30% conjugation efficiency (such as amino-PEG linkers known in the art; see Example 9).

Even more surprisingly, high conjugation efficiencies have been achieved with all peptide linkers. It is important to note that quantitative conjugation of native glycosylated antibodies with linkers comprising two or more payloads, in one step, was never reported. In this light, it has to be considered even more surprising that peptide linkers comprising two or more bulky payloads can be conjugated to native glycosylated antibodies with such high efficiencies.

Besides these exceptionally high conjugation efficiencies, the inventors found that antibodies that have been conjugated with the peptide linkers of the invention are active in vivo (see Example 11). Moreover, it was surprisingly found that antibodies conjugated with peptide linkers comprising payloads at their N-terminal and C-terminal ends have stronger anti-tumor activity than the benchmark antibody Enfortumab vedotin.

In a particular embodiment, the invention relates to a peptide linker comprising
a) an amino acid residue comprising a primary amine; and
b) two or more payloads;
   wherein each of the two or more payloads can be independently attached to:
i) an N-terminal end of the peptide linker,
ii) a C-terminal end of the peptide linker, or
iii) a side chain of an amino acid residue comprised in the peptide linker.

Accordingly, the invention relates to a peptide linker comprising two or more payloads covalently attached to a peptide moiety. In the prior art, two-step approaches for generating antibody-payload conjugates comprising a drug-to-antibody (DAR) ratio >4 have been postulated, wherein, in a first step, a linker comprising two reactive groups is conjugated to a glycosylated antibody by means of a microbial transglutaminase and, in a second step, payload molecules are coupled to the reactive groups comprised in the antibody-linker conjugate (WO 2019/057772 and WO 2015/191883). In particular, it has been considered difficult to conjugate linkers comprising bulky payloads, such as toxins, to native glycosylated antibodies in a single reaction step due to limited accessibility of the endogenous conjugation site Q295 and limited space in the substrate binding pocket of the transglutaminase. Despite these known difficulties, the inventors have surprisingly found that the peptide linkers of the present invention comprising two or more payloads can be conjugated to glycosylated antibodies with exceptionally high conjugation efficiencies.

A “peptide linker”, within the meaning of the present invention, is a molecule comprising at least two amino acid residues, wherein the two amino acid residues are coupled via a peptide bond. It is envisioned that the peptide linker is suitable as a substrate for a microbial transglutaminase. In particular, it is envisioned that the peptide linker is suitable for conjugation to a glutamine residue comprised in an antibody. For that, the peptide linker according to the invention has to comprise at least one amino acid residue comprising a primary amine.

Thus, in a particular embodiment, the invention relates to the peptide linker according to the invention, wherein the primary amine comprised in the amino acid residue is
a) a primary amine in a side chain of a lysine, a lysine derivative or a lysine mimetic; or
b) a primary amine comprised in an N-terminal amino acid residue having the structure NH2-(Y)—COOH.

That is, in a preferred embodiment, the amino acid residue comprising the primary amine is a lysine residue. In such embodiments, the peptide linker comprises a peptide moiety comprising at least one lysine residue.

However, the linker according to the invention may also comprise a lysine mimetic or a lysine derivative, provided that the lysine mimetic or lysine derivative comprises a free primary amine in the amino acid side chain.

In certain embodiments, the amino acid residue comprising the primary amine may be a lysine mimetic. The term “lysine mimetic”, as used herein, refers to a compound that has a structure different from lysine, but that has similar characteristics as lysine and may thus be used to replace lysine in a peptide or protein without significantly altering the function and/or structure of said peptide or protein. In certain embodiments, a lysine mimetic may differ from lysine in the length or composition of the aliphatic chain that connects the primary amine and the α-carbon atom. Thus, in certain embodiments, the lysine mimetic may be ornithine, homolysine or 2,7-diaminoheptanoic acid (exemplary linker containing an homolysine is shown in FIG. 23). In certain embodiments, the lysine mimetic may be a beta-amino acid, such as beta-homolysine.

In certain embodiments, the amino acid residue comprising the primary amine may be a lysine derivative. The term “lysine derivative”, as used herein, refers to a lysine or lysine mimetic, wherein one or more functional groups comprised in the lysine or lysine mimetic is (are) modified or substituted. Within the present invention, it is preferred that the amino group in the side chain of the lysine derivative is unmodified, such that is available for conjugation to a glutamine residue in a protein. Thus, the “lysine derivative” comprised in the peptide linker of the present invention preferably comprises a modified or substituted α-amino and/or a-carboxyl group.

In certain embodiments, the primary amine comprised in the amino acid residue may be a primary amine comprised in an N-terminal amino acid residue having the structure NH₂—(Y)—COOH.

In certain embodiments, the primary amine may be the α-amino group of an α-amino acid. The a-amino acid may be any proteinogenic α-amino acid, including alanine, arginine, asparagine, aspartic acid, cysteine, glutamic acid, glutamine, glycine, histidine, isoleucine, leucine, lysine, methionine, phenylalanine, serine, threonine, tryptophan, tyrosine and valine.

In a particularly preferred embodiment, the primary amine may be the α-amino group of a glycine residue. In such embodiments, it is preferred that the glycine residue is the N-terminal amino acid residue of the peptide linker, such that the α-amino group is available for conjugation to a glycine residue via a microbial transglutaminase.

The amino acid comprising the primary amine may be a non-canonical or a synthetic amino acid. A “non-canonical amino acid”, as used herein, may be any amino acid that is not part of the set of proteinogenic amino acids, but that can be obtained from a natural source. However, it has to be noted that some non-canonical amino acids may also be found in naturally occurring peptides and/or proteins. A “synthetic amino acid”, as used herein, may be any molecule that falls under the general definition of an amino acid (NH₂—(Y)—COOH), i.e., that comprises an amino group and a carboxyl group, but that is not found in nature. Thus, non-natural amino acids are preferably obtained by chemical synthesis. It is to be understood that the differentiation between a non-canonical amino acid and a synthetic amino acid may be uncertain in some instances. For example, an amino acid that is defined as a synthetic amino acid may be, at a later time point, identified in nature and thus reclassified as a non-canonical amino acid. The non-canonical or synthetic amino acid may be an α-, β-, γ-, δ-, or ε-amino acid.

In certain embodiments, the amino acid comprising the primary amine may have the structure NH₂—(Y)—COOH.

In certain embodiments, the moiety Y may comprise a carbon comprising framework of 1 to 200 atoms, optionally a carbon comprising framework of at least 10 atoms, e.g. 10 to 100 atoms or 20 to 100 atoms, substituted at one or more atoms, optionally wherein the carbon comprising framework is a linear hydrocarbon or comprises a cyclic group, a symmetrically or asymmetrically branched hydrocarbon, monosaccharide, disaccharide, linear or branched oligosaccharide (asymmetrically branched or symmetrically branched), other natural linear or branched oligomers (asymmetrically branched or symmetrically branched), or more generally any dimer, trimer, or higher oligomer (linear, asymmetrically branched or symmetrically branched) resulting from any chain-growth or step-growth polymerization process.

Y may further be any straight, branched and/or cyclic C₂₋₃₀ alkyl, C₂₋₃₀ alkenyl, C₂₋₃₀ alkynyl, C₂₋₃₀ heteroalkyl, C₂₋₃₀ heteroalkenyl, C₂₋₃₀ heteroalkynyl, optionally wherein one or more homocyclic aromatic compound radical or heterocyclic compound radical may be inserted; notably, any straight or branched C₂₋₅ alkyl, C₅ₛ₋₁₀ alkyl, C₁₁₋₂₀ alkyl, —O—C₁₋₅ alkyl, —O—C₅₋₁₀ alkyl, —O—C₁₁₋₂₀ alkyl, or (CH₂—CH₂—O—)₁₋₂₄ or (CH₂)ₓ₁—(CH₂—O—CH₂)₁₋₂₄—(CH₂)ₓ₂— group, wherein x1 and x2 are independently an integer selected among the range of 0 to 20, an amino acid, an oligopeptide, glycan, sulfate, phosphate, or carboxylate. In some embodiments, Y may comprise a C₂₋₆ alkyl group.

In a particular embodiment, the invention relates to the peptide linker according to the invention, wherein Y is —(R₂C)ₙ— and wherein n is an integer ranging from 1 to 20, from 1 to 15, from 1 to 10.

That is, Y may have the structure

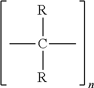

In certain embodiments, Y may be a substituted or unsubstituted alkyl or alkenyl chain. When Y is a substituted or unsubstituted alkenyl chain, it is to be understood that at least two R moieties attached to consecutive carbon molecules have to be absent.

The term “substituted alkyl”, as used herein, generally refers to an alkyl group with an additional group or groups attached to any carbon of the alkyl group. That is, the substituted alkyl may comprise the structure —(R₂C)ₙ—, wherein each R may independently be a hydrogen or a functional group such as an alkyl, lower alkyl, aryl, acyl, halogen, alkylhalo, hydroxy, amino, alkoxy, alkylamino, acylamino, acyloxy, aryloxy, aryloxyalkyl, mercapto, both saturated and unsaturated cyclic hydrocarbon, heterocycle, and other organic group.

In certain embodiments, the amino acid comprising the primary amine may have the structure NH₂—(Y)—COOH, wherein Y is —(R₂C)ₙ— and wherein n is 1, 2, 3, 4, 5, 6, 7, 8, 9, 10, 11, 12, 13, 14, 15, 16, 17, 18, 19 or 20. In certain embodiments, at least 1, 2, 3, 4 or 5 moieties R comprised in the structure —(R₂C)ₙ— may be a functional group such as an alkyl, lower alkyl, aryl, acyl, halogen, alkylhalo, hydroxy, amino, alkoxy, alkylamino, acylamino, acyloxy, aryloxy, aryloxyalkyl, mercapto, both saturated and unsaturated cyclic hydrocarbon, heterocycle, and other organic group.

In a particular embodiment, the invention relates to the peptide linker according to the invention, wherein at least one R moiety of each —(R₂C)— monomer is hydrogen.

That is, in certain embodiments, one R moiety of each —(R₂C)— monomer may be a hydrogen, while the other R moiety may be a functional group such as an alkyl, lower alkyl, aryl, acyl, halogen, alkylhalo, hydroxy, amino, alkoxy, alkylamino, acylamino, acyloxy, aryloxy, aryloxyalkyl, mercapto, both saturated and unsaturated cyclic hydrocarbon, heterocycle, and other organic group. Alternatively, one R moiety of each —(R₂C)— monomer may be hydrogen and the other R moiety may be absent (in case of alkenes). In certain embodiments, some —(R₂C)— monomers comprised in a moiety Y may comprise two hydrogen substituents and some —(R₂C)— monomers comprised in the same moiety Y may comprise one hydrogen substituent and one substituent R as defined herein.

In a particular embodiment, the invention relates to the peptide linker according to the invention, wherein both R moieties of each —(R₂C)— monomer are hydrogen.

In certain embodiments the structure —(R₂C)ₙ— may be an unsubstituted alkyl chain wherein all moieties R comprised in the structure —(R₂C)ₙ— are hydrogen atoms. That is, in certain embodiments, the structure —(R₂C)ₙ— may be a methyl, ethyl, propyl, butyl, pentyl, hexyl, heptyl, octyl, nonyl, decyl, undecyl, dodecyl, tridecyl, tetradecyl, pentadecyl, hexadecyl, heptadecyl or octadecyl group.

That is, in certain embodiments, the amino acid comprising the primary amine may have the structure NH₂—(Y)—COOH, wherein Y is —(CH₂)ₙ— and wherein n is an integer from 1 to 20. In certain embodiments, the amino acid comprising the primary amine may have the structure NH₂—(Y)—COOH, wherein Y is —(CH₂)ₙ— and wherein n is an integer from 1 to 15. in a certain embodiments, the amino acid comprising the primary amine may have the structure NH₂—(Y)—COOH, wherein Y is —(CH₂)ₙ— and wherein n is an integer from 1 to 10. in certain embodiments, the amino acid comprising the primary amine may have the structure NH₂—(Y)—COOH, wherein Y is —(CH₂)ₙ— and wherein n is an integer from 1 to 9. in certain embodiments, the amino acid comprising the primary amine may have the structure NH₂—(Y)—COOH, wherein Y is —(CH₂)ₙ— and wherein n is an integer from 1 to 8. in certain embodiments, the amino acid comprising the primary amine may have the structure NH₂—(Y)—COOH, wherein Y is —(CH₂)ₙ— and wherein n is an integer from 1 to 7. in certain embodiments, the amino acid comprising the primary amine may have the structure NH₂—(Y)—COOH, wherein Y is —(CH₂)ₙ— and wherein n is an integer from 1 to 6.

In certain embodiments, Y may have the structure —(CH₂)ₙ—, wherein n is 1. That is, in certain embodiments, the amino acid comprising the primary amine may be glycine.

In certain embodiments, Y may have the structure —(CH₂)ₙ—, wherein n is 2. That is, in certain embodiments, the amino acid comprising the primary amine may be P-alanine.

In certain embodiments, Y may have the structure —(CH₂)ₙ—, wherein n is 3. That is, in certain embodiments, the amino acid comprising the primary amine may be 4-aminobutyric acid.

In certain embodiments, Y may have the structure —(CH₂)ₙ—, wherein n is 4. That is, in certain embodiments, the amino acid comprising the primary amine may be 5-aminopentanoic acid. (Exemplary linker containing 5-aminopentanoic acid is shown in FIG. 24) In certain embodiments, Y may have the structure —(CH₂)ₙ—, wherein n is 5. That is, in certain embodiments, the amino acid comprising the primary amine may be 6-aminohexanoic acid.

In certain embodiments, Y may have the structure —(CH₂)ₙ—, wherein n is 6. That is, in certain embodiments, the amino acid comprising the primary amine may be 7-aminoheptanoic acid.

In certain embodiments, Y may have the structure —(CH₂)ₙ—, wherein n is 7. That is, in certain embodiments, the amino acid comprising the primary amine may be 8-aminooctanoic acid.

In certain embodiments, Y may have the structure —(CH₂)ₙ—, wherein n is 8. That is, in certain embodiments, the amino acid comprising the primary amine may be 9-aminononanoic acid.

In certain embodiments, Y may have the structure —(CH₂)ₙ—, wherein n is 9. That is, in certain embodiments, the amino acid comprising the primary amine may be 10-aminodecanoic acid.

In certain embodiments, Y may have the structure —(CH₂)ₙ—, wherein n is 10. That is, in certain embodiments, the amino acid comprising the primary amine may be 11-aminoundecanoic acid.

In certain embodiments, the amino acid comprising the primary amine may have the structure NH₂—(CH₂)ₙ—X—(CH₂)ₙ—COOH, wherein X is a substituted or unsubstituted alkyl or heteroalkyl chain and wherein n is an integer from 0-20, from 0-10 or from 0-6.

That is, in certain embodiments, the amino acid comprising the primary amine may have the structure NH₂—(CH₂)ₙ—X—COOH, wherein X is a substituted or unsubstituted alkyl or heteroalkyl chain and wherein n is an integer from 1-20, from 1-10 or from 1-6.

In certain embodiments, the amino acid comprising the primary amine may have the structure NH₂—X—(CH₂)ₙ—COOH, wherein X is a substituted or unsubstituted alkyl or heteroalkyl chain and wherein n is an integer from 1-20, from 1-10 or from 1-6.

In a preferred embodiment, the amino acid comprising the primary amine comprises at least one methylene group (CH₂). More preferably, the at least one methylene group is directly coupled to the primary amine. That is, the amino acid comprising the primary amine preferably comprises the structure NH₂—CH₂—.

In a preferred embodiment, the invention relates to the peptide linker according to the invention, wherein the primary amine comprised in the amino acid residue is
a) a primary amine in a side chain of a lysine, a lysine derivative or a lysine mimetic; or
b) a primary amine comprised in an N-terminal amino acid residue having the structure NH2-(CH₂)ₙ—COOH, wherein n is an integer ranging from 1 to 10.

In certain embodiments, a payload is attached to the N-terminal end of the peptide linker. In such embodiments, it is preferred that the primary amine comprised in the amino acid residue is a primary amine in a side chain of a lysine, a lysine derivative or a lysine mimetic; more preferably a primary amine in a side chain of a lysine residue.

In a particular embodiment, the invention relates to the peptide linker according to the invention, wherein the linker comprises not more than 25, 20, 15, 14, 13, 12, 11, 10, 9, 8, 7, 6, 5, 4 amino acid residues.

The peptide linker according to the invention preferably comprises at least two amino acid residues and not more than 25 amino acid residues. In a preferred embodiment, all amino acid residues comprised in the peptide linker according to the invention form a single peptide. However, it is to be understood that the peptide linker may comprise two or more peptide moieties. For example, in certain embodiments, the peptide linker may comprise two peptide moieties, wherein the two peptide moieties are connected to each other covalently, but not by a peptide bond. Examples of such peptide linkers will be given further below.

In a particular embodiment, the invention relates to the peptide linker according to the invention, wherein the net charge of the linker is neutral or positive.

The net charge of a peptide is usually calculated at neutral pH (7.0). In the simplest approach, the net charge is determined by adding the number of positively charged amino acid residues (Arg, Lys and His) and the number of negatively charged ones (Asp and Glu) and calculate the difference of the two groups. In cases where the linker comprises non-canonical amino acids or amino acid derivatives comprising a charged functional group, the skilled person is capable of calculating the net charge at neutral pH accordingly.

In certain embodiments, the payloads may also contribute to the net charge of the linker. However, the skilled person is aware of methods to calculate the net charge of the entire linker, including any payloads, preferably at neutral pH (7.0).

In certain embodiments, the net charge of a peptide linker is calculated solely based on the amino acid residues comprised in the linker, including amino acid mimetics and amino acid derivatives. Thus, in a particular embodiment, the invention relates to the peptide linker according to the invention, wherein the net charge of the amino acid residues comprised in the peptide linker is neutral or positive.

In a particular embodiment, the invention relates to the peptide linker according to the invention, wherein the linker comprises no negatively-charged amino acid residues.

That is, the linker may be free of negatively charged amino acid residues, including negatively-charged amino acid mimetics and amino acid derivatives. A negatively charged amino acid residue is an amino acid, amino acid mimetic or amino acid derivative which carries a negative charge at neutral pH (7.0).

Negatively charged canonical amino acids are glutamic acid and aspartic acid. However, negatively charged non-canonical amino acids, amino acid mimetics and amino acid derivatives are known in the art.

It has to be noted that the peptide linker according to the invention may comprise one or more glutamate or aspartate residue. However, it is preferred that the carboxyl group comprised in the aspartate or glutamate side chain is coupled to a payload.

In a particular embodiment, the invention relates to the peptide linker according to the invention, wherein the linker comprises at least one positively-charged amino acid residue.

In certain embodiments, the peptide linker comprises a positively charged lysine residue, which provides the primary amine for the transglutaminase-mediated conjugation to an antibody. However, it is preferred herein that the peptide linker comprises at least one additional positively charged amino acid. The additional positively charged amino acid may be a canonical amino acid residue, such as arginine or histidine. However, the additional positively charged amino acid may also be a non-canonical amino acid.

In a particular embodiment, the invention relates to the peptide linker according to the invention, wherein the linker comprises at least one arginine residue.

It has been demonstrated herein that linkers comprising an arginine residue can be conjugated to glycosylated antibodies with high efficiency. Thus, it is preferred herein that the peptide linker according to the invention comprises at least one arginine residue. It is to be noted that the arginine residue may also be replaced by an arginine mimetic or arginine derivative.

The arginine residue may be located at any position of the peptide linker. In certain embodiments, the arginine residue is adjacent to the amino acid residue comprising the primary amine. In certain embodiments, the arginine residue is coupled to the N-terminus of the amino acid comprising the primary amine, i.e., a lysine residue, a lysine mimetic or a lysine derivative (e.g., RK motif). In certain embodiments, the arginine residue is coupled to the C-terminus of the amino acid comprising the primary amine, i.e., a lysine residue, a lysine mimetic or a lysine derivative (e.g., KR motif). In certain embodiments, the arginine residue is coupled to the amino acid comprising the primary amine, i.e., a lysine residue, a lysine mimetic or a lysine derivative, via another amino acid residue, preferably an alanine residue (KAR or RAK motif). In certain embodiments, the peptide linker comprises an arginine and a histidine residue.

In a particular embodiment, the invention relates to the peptide linker according to the invention, wherein the linker comprises at least one histidine residue.

It has been demonstrated herein that linkers comprising a histidine residue can be conjugated to glycosylated antibodies with high efficiency. Thus, it is preferred herein that the peptide linker according to the invention comprises at least one histidine residue. It is to be noted that the histidine residue may also be replaced by a histidine mimetic or histidine derivative.

The histidine residue may be located at any position of the peptide linker. In certain embodiments, the histidine residue is adjacent to the amino acid residue comprising the primary amine. In certain embodiments, the histidine residue is coupled to the N-terminus of the amino acid comprising the primary amine, i.e., a lysine residue, a lysine mimetic or a lysine derivative (e.g., HK motif). In certain embodiments, the histidine residue is coupled to the C-terminus of the amino acid comprising the primary amine, i.e., a lysine residue, a lysine mimetic or a lysine derivative (e.g., KH motif). In certain embodiments, the histidine residue is coupled to the amino acid comprising the primary amine, i.e., a lysine residue, a lysine mimetic or a lysine derivative, via another amino acid residue, preferably an alanine residue (KAH or HAK motif). In certain embodiments, the peptide linker comprises a histidine and an arginine residue.

In a particular embodiment, the invention relates to the peptide linker according to the invention, wherein the linker comprises the sequence motif RK.

It has been shown by the inventors that peptide linkers comprising the sequence motif RK (arginyl-lysyl) can be conjugated to glycosylated antibodies with exceptionally high efficiency, even if the linker comprises two or more payloads. It is to be understood that the lysine residue comprised in the RK motif contains the primary amine via which the peptide linker is conjugated to a glutamine residue comprised in the antibody. That is, the lysine residue comprised in the RK motif is preferably the amino acid comprising the primary amine.

It is preferred herein that the motif RK consists of the amino acids arginine and lysine. However, it is to be understood that the arginine and/or the lysine residue may be substituted with an arginine mimetic/derivative and/or a lysine mimetic/derivative.

That is, in certain embodiments, the motif RK may comprise an arginine mimetic. The term “arginine mimetic”, as used herein, refers to a compound that has a structure that is different from arginine, but that has similar characteristics as arginine and may thus be used to replace arginine in a peptide or protein without significantly altering the function and/or structure of said peptide or protein. An arginine mimetic may differ from arginine in length or composition of the aliphatic chain that connects the guanidino group and the a-carbon atom. Alternatively, or in addition, arginine mimetics may differ from arginine in the guanidino group itself. That is, the arginine mimetic may comprise a functional group with similar physicochemical properties as the guanidino group. In certain embodiments, the arginine mimetic may be homoarginine, 2-amino-3-guanidino-propionic acid, β-ureidoalanine or citrulline.

In certain embodiments, the motif RK may comprise an arginine derivative. The term “arginine derivative”, as used herein, refers to an arginine or arginine mimetic, wherein one or more functional groups comprised in the arginine or arginine mimetic is (are) modified or substituted. An arginine derivative may be arginine or an arginine mimetic, wherein the guanidino group is substituted or modified. In certain embodiments, the arginine derivative may be ω-methylarginine. In embodiments, where the residue R is located in the N-terminal position of the linker, R may be an arginine derivative wherein the α-amino group is modified or substituted. In certain embodiments the α-amino group of the arginine or arginine mimetic may be acetylated.

In certain embodiments, the motif RK may comprise a lysine mimetic or lysine derivative as defined elsewhere herein.

In certain embodiments, the motif RK may comprise a lysine mimetic/derivative and an arginine mimetic/derivative.

In certain embodiments, the lysine residue, or the lysine mimetic or lysine derivative, may be separated from the arginine residue, or the arginine mimetic or arginine derivative, by one amino acid residue. That is, the peptide linker of the invention may comprise the sequence motif RXK or KXR, wherein X may be any amino acid. In a preferred embodiment, the lysine residue, or the lysine mimetic or lysine derivative, may be separated from the arginine residue, or the arginine mimetic or arginine derivative, by an alanine residue. That is, the peptide linker of the invention may comprise the sequence motif RAK or KAR. It has been demonstrated in Example 10 that a linker comprising the sequence motif KAR can be conjugated to glycosylated antibodies with exceptionally high conjugation efficiency.

In a particular embodiment, the invention relates to the peptide linker according to the invention, wherein the linker comprises the sequence motif HK.

It has been shown by the inventors that peptide linkers comprising the sequence motif HK (histidyl-lysyl) can be conjugated to glycosylated antibodies with very high efficiency, even if the linker comprises two or more payloads (e.g., Example 3). It is to be understood that the lysine residue comprised in the HK motif contains the primary amine via which the peptide linker is conjugated to a glutamine residue comprised in the antibody. That is, the lysine residue comprised in the HK motif is preferably the amino acid comprising the primary amine.

It is preferred herein that the motif HK consists of the amino acids histidine and lysine. However, it is to be understood that the histidine and/or the lysine residue may be substituted with a histidine mimetic/derivative and/or a lysine mimetic/derivative.

That is, in certain embodiments, the motif HK may comprise a histidine mimetic. The term “histidine mimetic”, as used herein, refers to a compound that has a structure that is different from histidine, but that has similar characteristics as histidine and may thus be used to replace histidine in a peptide or protein without significantly altering the function and/or structure of said peptide or protein. A histidine mimetic may differ from histidine in length or composition of the aliphatic chain that connects the imidazole group and the a-carbon atom. Alternatively, or in addition, histidine mimetics may differ from histidine in the imidazole group itself. That is, the histidine mimetic may comprise a functional group with similar physicochemical properties as the imidazole group. In certain embodiments, the histidine mimetic may be homohistidine.

In certain embodiments, the motif HK may comprise a histidine derivative. The term “histidine derivative”, as used herein, refers to a histidine or histidine mimetic, wherein one or more functional groups comprised in the histidine or histidine mimetic is (are) modified or substituted. A histidine derivative may be histidine or a histidine mimetic, wherein the imidazole group is substituted or modified. In embodiments, where the residue H is located in the N-terminal position of the linker, H may be a histidine derivative wherein the α-amino group is modified or substituted. In certain embodiments the α-amino group of the histidine or histidine mimetic may be acetylated.

In certain embodiments, the motif HK may comprise a lysine mimetic or lysine derivative as defined elsewhere herein.

In certain embodiments, the motif HK may comprise a lysine mimetic/derivative and a histidine mimetic/derivative.

In certain embodiments, the lysine residue, or the lysine mimetic or lysine derivative, may be separated from the arginine residue, or the arginine mimetic or arginine derivative, by one amino acid residue. That is, the peptide linker of the invention may comprise the sequence motif HXK or KXH, wherein X may be any amino acid. In a preferred embodiment, the lysine residue, or the lysine mimetic or lysine derivative, may be separated from the arginine residue, or the arginine mimetic or arginine derivative, by an alanine residue. That is, the peptide linker of the invention may comprise the sequence motif HAK or KAH.

In a particular embodiment, the invention relates to the peptide linker according to the invention, wherein the linker comprises any one of the amino acid sequences set forth in SEQ ID NO:1-29 or 82-93.

That is, the peptide linker according to the invention may comprise any one of the amino acid sequences set forth in SEQ ID NOs: 1-29 or 82-93.

That is in certain embodiments, the peptide linker may comprise the peptide sequence RKAA (SEQ ID NO:1). Several linkers comprising the sequence RKAA have been shown herein (see FIGS. 1, 2, 3, 4, 13, 14, 17, 20, 21, 30, 35, 36, 37 and 39). Preferably, one or more payloads are attached to the N- and/or C-terminus of the peptide RKAA.

In certain embodiments, the peptide linker may comprise the peptide sequence ARK (SEQ ID NO:2). A linker comprising the sequence ARK is exemplified in FIG. 5. Preferably, one or more payloads are attached to the N- and/or C-terminus of the peptide ARK.

In certain embodiments, the peptide linker may comprise the peptide sequence RKARA (SEQ ID NO:3). A linker comprising the sequence RKARA is exemplified in FIG. 6. Preferably, one or more payloads are attached to the N- and/or C-terminus of the peptide RKARA.

In certain embodiments, the peptide linker may comprise the peptide sequence RKAAAA (SEQ ID NO:4). A linker comprising the sequence RKAAAA is exemplified in FIG. 7. Preferably, one or more payloads are attached to the N- and/or C-terminus of the peptide RKAAAA.

In certain embodiments, the peptide linker may comprise the peptide sequence RKAAAAAA (SEQ ID NO:5). A linker comprising the sequence RKAAAAAA is exemplified in FIG. 8. Preferably, one or more payloads are attached to the N- and/or C-terminus of the peptide RKAAAAAA.

In certain embodiments, the peptide linker may comprise the peptide sequence RKAASGSG (SEQ ID NO:6). A linker comprising the sequence RKAASGSG is exemplified in FIG. 9. Preferably, one or more payloads are attached to the N- and/or C-terminus of the peptide RKAASGSG.

In certain embodiments, the peptide linker may comprise the peptide sequence RKHA (SEQ ID NO:7). A linker comprising the sequence RKHA is exemplified in FIG. 10. Preferably, one or more payloads are attached to the N- and/or C-terminus of the peptide RKHA.

In certain embodiments, the peptide linker may comprise the peptide sequence RKHAAA (SEQ ID NO:8). A linker comprising the sequence RKHAAA is exemplified in FIG. 11. Preferably, one or more payloads are attached to the N- and/or C-terminus of the peptide RKHAAA.

In certain embodiments, the peptide linker may comprise the peptide sequence GGR (SEQ ID NO:9). A linker comprising the sequence GGR is exemplified in FIG. 15. Preferably, one or more payloads are attached to the N- and/or C-terminus of the peptide GGR.

In certain embodiments, the peptide linker may comprise the peptide sequence GGRG (SEQ ID NO:10).

A linker comprising the sequence GGRG is exemplified in FIGS. 16 and 18. Preferably, one or more payloads are attached to the N- and/or C-terminus of the peptide GGRG.

In certain embodiments, the peptide linker may comprise the peptide sequence EARKAA (SEQ ID NO:11). A linker comprising the sequence EARKAA is exemplified in FIG. 19. Preferably, one or more payloads are attached to the N- and/or C-terminus of the peptide EARKAA. In addition, it is preferred that one or more payloads are attached to the side chain of the glutamate residue. It is to be understood that when an amine comprising payload is attached to the side chain of the glutamate residue, the peptide sequence of the linker may also be viewed as QARKAA (SEQ ID NO:84).

In certain embodiments, the peptide linker may comprise the peptide sequence RKAEA (SEQ ID NO:12). A linker comprising the sequence RKAEA is exemplified in FIG. 22. Preferably, one or more payloads are attached to the N- and/or C-terminus of the peptide RKAEA. In addition, it is preferred that one or more payloads are attached to the side chain of the glutamate residue. It is to be understood that when an amine comprising payload is attached to the side chain of the glutamate residue, the peptide sequence of the linker may also be viewed as RKAQA (SEQ ID NO:85).

In certain embodiments, the peptide linker may comprise the peptide sequence HKA (SEQ ID NO:13). A linker comprising the sequence HKA is exemplified in FIG. 12. Preferably, one or more payloads are attached to the N- and/or C-terminus of the peptide HKA.

In certain embodiments, the peptide linker may comprise the peptide sequence RhKAA (SEQ ID NO:14). A linker comprising the sequence RhKAA is exemplified in FIG. 23. Preferably, one or more payloads are attached to the N- and/or C-terminus of the peptide RhKAA.

In certain embodiments, the peptide linker may comprise the peptide sequence XGRG (SEQ ID NO:15), wherein X has the structure NH2-(CH₂)ₙ—COOH, wherein n is an integer from 1-20, preferably from 1-10. A linker comprising the sequence XGRG is exemplified in FIG. 24. Preferably, one or more payloads are attached to the C-terminus of the peptide XGRG.

In certain embodiments, the peptide linker may comprise the peptide sequence RKVCit (SEQ ID NO:16). A linker comprising the sequence RKVCit is exemplified in FIG. 25. Preferably, one or more payloads are attached to the N- and/or C-terminus of the peptide RKVCit.

In certain embodiments, the peptide linker may comprise the peptide sequence RKAR (SEQ ID NO:17). A linker comprising the sequence RKAR is exemplified in FIG. 26. Preferably, one or more payloads are attached to the N- and/or C-terminus of the peptide RKAR.

In certain embodiments, the peptide linker may comprise the peptide sequence RKVA (SEQ ID NO:18). A linker comprising the sequence RKVA is exemplified in FIG. 27. Preferably, one or more payloads are attached to the N- and/or C-terminus of the peptide RKVA.

In certain embodiments, the peptide linker may comprise the peptide sequence KAR (SEQ ID NO:19). A linker comprising the sequence KAR is exemplified in FIGS. 28 and 42. Preferably, one or more payloads are attached to the N- and/or C-terminus of the peptide KAR.

In certain embodiments, the peptide linker may comprise the peptide sequence RKEAA (SEQ ID NO:20). A linker comprising the sequence RKEAA is exemplified in FIG. 29. Preferably, one or more payloads are attached to the N- and/or C-terminus of the peptide RKEAA. In addition, it is preferred that one or more payloads are attached to the side chain of the glutamate residue. It is to be understood that when an amine comprising payload is attached to the side chain of the glutamate residue, the peptide sequence of the linker may also be viewed as RKQAA (SEQ ID NO:86).

In certain embodiments, the peptide linker may comprise the peptide sequence RKDA (SEQ ID NO:82). A linker comprising the sequence RKDA is exemplified in FIG. 43. Preferably, one or more payloads are attached to the N- and/or C-terminus of the peptide RKDA. In addition, it is preferred that one or more payloads are attached to the side chain of the aspartate residue. It is to be understood that when an amine comprising payload is attached to the side chain of the aspartate residue, the peptide sequence of the linker may also be viewed as RKNA (SEQ ID NO:83).

In certain embodiments, the peptide linker may comprise the peptide sequence ERKAA (SEQ ID NO:21). A linker comprising the sequence ERKAA is exemplified in FIG. 30. Preferably, one or more payloads are attached to the N- and/or C-terminus of the peptide ERKAA. In addition, it is preferred that one or more payloads are attached to the side chain of the glutamate residue. It is to be understood that when an amine comprising payload is attached to the side chain of the glutamate residue, the peptide sequence of the linker may also be viewed as QRKAA (SEQ ID NO:87).

In certain embodiments, the peptide linker may comprise the peptide sequence RKAH (SEQ ID NO:22). A linker comprising the sequence RKAH is exemplified in FIG. 31. Preferably, one or more payloads are attached to the N- and/or C-terminus of the peptide RKAH.

In certain embodiments, the peptide linker may comprise the peptide sequence RKAN (SEQ ID NO:23). A linker comprising the sequence RKAN is exemplified in FIG. 32. Preferably, one or more payloads are attached to the N- and/or C-terminus of the peptide RKAN.

In certain embodiments, the peptide linker may comprise the peptide sequence RKGGFG (SEQ ID NO:24). A linker comprising the sequence RKGGFG is exemplified in FIG. 33. Preferably, one or more payloads are attached to the N- and/or C-terminus of the peptide RKGGFG.

In certain embodiments, the peptide linker may comprise the peptide sequence RKGP (SEQ ID NO:25). A linker comprising the sequence RKGP is exemplified in FIG. 34. Preferably, one or more payloads are attached to the N- and/or C-terminus of the peptide RKGP.

In certain embodiments, the peptide linker may comprise the peptide sequence KRKAA (SEQ ID NO:26). A linker comprising the sequence KRKAA is exemplified in FIG. 36. Preferably, one or more payloads are attached to the N- and/or C-terminus of the peptide KRKAA. In addition, it is preferred that one or more payloads are attached to the side chain of one of the lysine residues, preferably the N-terminal lysine residue.

In certain embodiments, the peptide linker may comprise the peptide sequence SRKAA (SEQ ID NO:27). A linker comprising the sequence SRKAA is exemplified in FIG. 37. Preferably, one or more payloads are attached to the N- and/or C-terminus of the peptide SRKAA. In addition, it is preferred that one or more payloads are attached to the side chain of the serine residue.

In certain embodiments, the peptide linker may comprise the peptide sequence DDRKAA (SEQ ID NO:28). A linker comprising the sequence DDRKAA is exemplified in FIG. 39. Preferably, one or more payloads are attached to the N- and/or C-terminus of the peptide DDRKAA. In addition, it is preferred that one or more payloads are attached to the side chains of the aspartate residues. It is to be understood that when an amine comprising payload is attached to a side chain of an aspartate residue, the peptide sequence of the linker may also be viewed as DNRKAA (SEQ ID NO:88), NDRKAA (SEQ ID NO:89) or NNRKAA (SEQ ID NO:90).

In certain embodiments, the peptide linker may comprise the peptide sequence EERKValCit (SEQ ID NO:29). A linker comprising the sequence EERKValCit is exemplified in FIG. 40. Preferably, one or more payloads are attached to the N- and/or C-terminus of the peptide EERKValCit. In addition, it is preferred that one or more payloads are attached to the side chains of the glutamate residues. It is to be understood that when an amine comprising payload is attached to a side chain of a glutamate residue, the peptide sequence of the linker may also be viewed as EQRKValCit (SEQ ID NO:91), QERKValCit (SEQ ID NO:92) or QQRKValCit (SEQ ID NO:93).

In certain embodiments, the peptide linker is any one of the linkers shown in FIG. 1-40 or 42-43.

In a particular embodiment, the invention relates to the peptide linker according to the invention, wherein the linker comprises between 2 and 4 payloads.

The peptide linker according to the invention may be used for the generation of antibody-payload conjugates having a payload-to-antibody ratio of 4 or higher by means of a microbial transglutaminase. Native glycosylated antibodies have a single conjugation site at glutamine residue 295 (Q295) of the heavy chain. Since antibodies comprise two heavy chains, conjugating a linker with two payloads to each of the glutamine residues results in an antibody-payload conjugate comprising 4 payloads. Analogously, conjugating a linker with three or four payloads to each of the glutamine residues results in an antibody-payload conjugate comprising 6 or 8 payloads, respectively. Thus, in certain embodiments, the peptide linker according to the invention comprises 2, 3 or 4 payloads.

The inventors identified different ways to couple two or more payloads to a peptide linker. In certain embodiments, two payloads may be coupled to the C-terminal end of a peptide linker (see FIGS. 1, 2, 4, 5, 6, 7, 8, 9, 10, 11, 12, 13, 15, 16, 17, 18, 19, 20, 21, 23, 24, 25, 28, 36, 37 and 40). In other embodiments two payloads may be coupled to the N-terminal end of a peptide linker (see FIG. 20). In yet another embodiment, one or two payloads may be coupled each to the N-terminal end of a peptide linker and to the C-terminal end of a peptide linker (see FIGS. 3, 14, 20, 21, 22, 26, 27, 31, 32, 33, 34, and 38).

In embodiments where no payload is attached to the N- or C-terminus of a peptide linker, it is preferred that the respective terminus is modified. That is, the N-terminus of a peptide linker is preferably acetylated and the C-terminus of a peptide linker is preferably amidated.

In addition to coupling payloads to the terminal ends of a peptide linker, one or more payloads may also be coupled to amino acid side chains (see FIGS. 19, 22, 29, 30, 35, 36, 37, 39 and 40). The skilled person is aware of amino acid residues having functional groups in their amino acid side chains that allow for coupling of a payload. Amino acids having functional groups in their side chains include, but are not limited to, those described by deGruiter et al in Biochemistry 2017, 56, 30, 3863-3873. Additionally, payloads may also be coupled to the side chain of non-canonical amino acids, including but not limited to pAcF, CpK, pAMF, SCpHK, AzK, Sec.

That is, in a particular embodiment, the invention relates to the peptide linker according to the invention, wherein at least one payload is attached to a side chain of a glutamate, aspartate, tryptophan, cysteine, lysine, tyrosine, serine or threonine residue comprised in the peptide linker.

In a particular embodiment, one or two payloads may be attached to the carboxylic acid of a glutamate or aspartate side chain (see FIGS. 19, 22, 29, 30, 39 and 40).

In a particular embodiment, one or two payloads may be attached to the amine of a lysine side chain (see FIG. 36).

In a particular embodiment, one or two payloads may be attached to the thiol of a cysteine side chain (see FIG. 35).

In a particular embodiment, one or two payloads may be attached to the hydroxyl of a serine, threonine, or tyrosine side chain (see FIG. 37).

The payloads may be directly coupled to the peptide linker. For example, an amine-comprising payload may be coupled to the C-terminal end of a peptide linker via an isopeptide bond (see FIG. 27). Similarly, a carboxyl-comprising payload may be coupled to the N-terminal end of a peptide linker via an isopeptide bond (see FIG. 32) or a thiol-comprising payload may be coupled to the side chain of a cysteine residue comprised in the peptide linker.

However, it is preferred herein that the payloads are coupled to the peptide linker via a chemical linker. In particular when two payloads are to be attached either to the N-terminal end or the C-terminal end of the peptide linker, the use of a chemical linker between the two payloads and the N- or C-terminal end is preferred.

Accordingly, in a particular embodiment, the invention relates to the peptide linker according to the invention, wherein at least one of the two or more payloads is attached to the peptide linker via a chemical linker.

Within the present invention, it is preferred that at least one of the two or more payloads is coupled to the peptide linker via a chemical linker. However, even more preferably, all payloads are coupled to the peptide linker via a chemical linker.

A chemical linker can have various purposes. In certain embodiments, the chemical linker merely functions as an “adapter” to couple one payload to a peptide linker. For example, a chemical linker comprising an amine group may be used for coupling a payload to the C-terminal end of a peptide linker via an amide bond. In such embodiments, it is preferred that the chemical linker comprises one or more functional groups other than the amine to allow coupling of the payloads to the chemical linker via these additional functional groups.

In certain embodiments, the chemical linker functions as an “amplifier moiety” to couple several payloads to a peptide linker. For example, a chemical linker comprising a disubstituted amine may be used as a dendron to attach two payloads (Exemplary linker containing amplifiers are shown in FIGS. 36 and 37). Another example of an amplifier is the 2,6-bis-(hydroxymethyl)-p-cresol moiety (as shown in FIG. 28).

Similarly, chemical linkers comprising a carboxyl group may be used for coupling one or more payload to the N-terminal end of a peptide linker via an amide bond. For example, a dicarboxylic acid molecule may be used for coupling an amine-comprising payload to the N-terminal end of a peptide (see FIGS. 22, 31, and 33).

Further, chemical linkers comprising a compatible functional group may be used for coupling a payload to an amino acid side chain comprising in a peptide linker.

In any of the embodiments disclosed above, the skilled person is capable of identifying a chemical linker that is suitable for coupling a payload to a peptide linker, whether the chemical serves as an “adapter” or an “amplifier moiety”. That is, the skilled person is able to identify a linker having the functional groups that are required for coupling a payload of interest to a functional group comprised in the peptide linker.

However, the chemical linker may not only function as an adapter between the payload(s) and the peptide linker, but also fulfill other functions.

That is, in certain embodiments, the invention relates to the peptide linker according to the invention, wherein the chemical linker is an enzymatically and/or chemically cleavable linker.

The cleavable linker may be any enzymatically and/or chemically cleavable linker known in the art, including, but not limited to, those described by Bargh et al (Chem. Soc. Rev., 2019, 48, 4361), which is fully incorporated herein by reference.

Cleavable linkers have the advantage that the release of the payloads from the antibody can be controlled and/or facilitated. For example, one or more payloads may be coupled to the peptide linker via an enzymatically and/or chemically cleavable chemical linker.

In certain embodiments, the chemical linker is cleavable in vivo. Cleavable linkers may include chemically or enzymatically unstable or degradable linkages. Cleavable linkers generally rely on biological processes to liberate the payload, such as reduction in the cytoplasm, exposure to acidic conditions in the lysosome, or cleavage by specific proteases or other enzymes within, or outside, the cell. Cleavable linkers generally incorporate one or more chemical bonds that are either chemically or enzymatically cleavable. In certain embodiments, a linker comprises a chemically labile group such as hydrazone and/or disulfide groups. Linkers comprising chemically labile groups exploit differential properties between the plasma and some cytoplasmic compartments. The intracellular conditions to facilitate payload release for hydrazone containing linkers are the acidic environment of endosomes and lysosomes, while the disulfide containing linkers are reduced in the cytosol, which contains high thiol concentrations, e.g., glutathione. In certain embodiments, the plasma stability of a linker comprising a chemically labile group may be increased by introducing steric hindrance using substituents near the chemically labile group.

Acid-labile groups, such as hydrazone or carbonate, remain intact during systemic circulation in the blood's neutral pH environment (pH 7.3-7.5) and undergo hydrolysis and release the payload once the ADC is internalized into mildly acidic endosomal (pH 5.0-6.5) and lysosomal (pH 4.5-5.0) compartments of the cell. This pH dependent release mechanism has been associated with nonspecific release of the payload. To increase the stability of the hydrazone group of the linker, the linker may be varied by chemical modification, e.g., substitution, allowing tuning to achieve more efficient release in the lysosome with a minimized loss in circulation. Hydrazone- or carbonate-containing linkers may contain additional cleavage sites, such as additional acid-labile cleavage sites and/or enzymatically labile cleavage sites. Exemplary linker with a carbonate acid-labile group is shown in FIG. 34.

Other acid-labile groups that may be included in chemical linkers include cis-aconityl-containing linkers. Cis-Aconityl chemistry uses a carboxylic acid juxtaposed to an amide bond to accelerate amide hydrolysis under acidic conditions.

Cleavable chemical linkers may also include a disulfide group. Disulfides are thermodynamically stable at physiological pH and are designed to release the payload upon internalization inside cells, wherein the cytosol provides a significantly more reducing environment compared to the extracellular environment. Scission of disulfide bonds generally requires the presence of a cytoplasmic thiol cofactor, such as (reduced) glutathione (GSH), such that disulfide-containing linkers are reasonably stable in circulation, selectively releasing the payload in the cytosol. The intracellular enzyme protein disulfide isomerase, or similar enzymes capable of cleaving disulfide bonds, may also contribute to the preferential cleavage of disulfide bonds inside cells. GSH is reported to be present in cells in the concentration range of 0.5-10 mM compared with a significantly lower concentration of GSH or cysteine, the most abundant low-molecular weight thiol, in circulation at approximately 5 μM. Tumor cells, where irregular blood flow leads to a hypoxic state, result in enhanced activity of reductive enzymes and therefore even higher glutathione concentrations. In certain embodiments, the in vivo stability of a disulfide-containing linker may be enhanced by chemical modification of the linker, e.g., use of steric hinderance adjacent to the disulfide bond. Exemplary linker with a disulfide group is shown in FIG. 35.

Another type of cleavable linker that may be used is a chemical linker that is specifically cleaved by an enzyme. Such linkers are typically peptide-based or include peptidic regions that act as substrates for enzymes. Peptide based linkers tend to be more stable in plasma and extracellular milieu than chemically labile linkers. Peptide bonds generally have good serum stability, as lysosomal proteolytic enzymes have very low activity in blood due to endogenous inhibitors and the unfavorably high pH value of blood compared to lysosomes. Release of a payload from an antibody occurs specifically due to the action of lysosomal proteases, e.g., cathepsin, legumain, and plasmin. These lysosomal proteases may be present at elevated levels within certain tumor cells, but can also be found extracellularly, in the tumor microenvironment. Peptide-based linkers could also be cleaved by non-lysosomal extracellular proteases such as matrix metalloproteinases. Non-peptide-based linkers could also be specifically cleaved by glycosidases.

In exemplary embodiments, the cleavable peptide is selected from tetrapeptides such as Gly-Phe-Leu-Gly (SEQ ID NO:30), Ala-Leu-Ala-Leu (SEQ ID NO:31), Gly-Gly-Phe-Gly (SEQ ID NO:32) or dipeptides such as Ala-Ala, Ala-Arg, Val-Cit, Val-Ala, Met-(D)Lys, Asn-(D)Lys, Val-(D)Asp, Phe-Lys, Ile-Val, Asp-Val, His-Val, NorVal-(D)Asp, Ala-(D)Asp, Met-Lys, Asn-Lys, lie-Pro, Me3Lys-Pro, PhenylGly-(D)Lys, Met-(D)Lys, Asn-(D)Lys, Pro-(D)Lys, Met-(D)Lys, Asn-(D)Lys, Met-(D)Lys, Asn-(D)Lys. In certain embodiments, dipeptides are preferred over longer polypeptides due to hydrophobicity of the longer peptides. That is, linkers comprising an amino acid as set forth in SEQ ID NO:1-29 or 82-93 may further comprise any of the dipeptide or tetrapeptide motifs listed above. Preferably, the dipeptide or tetrapeptide motifs listed above are directly coupled to a payload or are coupled to a payload via a self-immolative spacer.

Enzymatically cleavable linkers may include a self-immolative spacer to spatially separate the payload from the site of enzymatic cleavage. The direct attachment of a payload to a peptide linker can result in proteolytic release of an amino acid adduct of the payload, thereby impairing its activity. The use of a self-immolative spacer allows for the elimination of the fully active, chemically unmodified payload upon amide or glycosidic bond hydrolysis.

In certain embodiments, the invention relates to the peptide linker according to the invention, wherein the self-immolative linker comprises
a) a p-aminobenzyl alcohol moiety; or
b) a 2,4-bis(hydroxymethyl)aniline moiety; or
c) a p-aminobenzyl quaternary ammonium; or
d) an ethylenediamine-based moiety; or
e) an (aminomethyl)pyrrolidine-based moiety; or
f) an aminomethyl-based moiety.

One self-immolative spacer is the bifunctional para-aminobenzyl alcohol group, which is linked to the peptide through the amino group, forming an amide bond, while amine-containing drugs may be attached through carbamate functionalities to the benzylic hydroxyl group of the linker (PABC). The resulting prodrugs are activated upon protease-mediated cleavage, leading to a 1,6-elimination reaction releasing the unmodified drug, carbon dioxide, and remnants of the linker group. Heterocyclic variants of this self-immolative group have also been described. See for example, U.S. Pat. No. 7,989,434, incorporated herein by reference. The para-aminobenzyl alcohol moiety may also be used to link a phenol- or hydroxyl-containing payload through the formation of a carbonate (see FIG. 34). The para-aminobenzyl moiety may also be used to link a tertiary- or heteroaryl-amine-containing payload through the formation of a quaternary ammonium (PABQ) (see FIG. 32). That is, in certain embodiments, the invention relates to the peptide linker according to the invention, wherein the quaternary ammonium cation comprised in the p-aminobenzyl quaternary ammonium originates from an amine comprised in the payload. Preferably, the amine comprised in the payload is a tertiary amine or a heteroaryl-amine.

Another self-immolative spacer is the 2,4-bis(hydroxymethyl)aniline group, which is linked to the peptide through the amino group, forming an amide bond, while amine-containing drugs may be attached through two carbamate functionalities via the two benzylic hydroxyl groups of the linker. The resulting prodrugs are activated upon protease-mediated cleavage, leading to payload release via successive 1,6- and 1,4-elimination processes.

For hydroxyl-containing drugs, suitable self-immolative spacers include, but are not limited to, ethylenediamine-based carbamate (EDA) (se FIG. 22 31), (aminomethyl)pyrrolidine-based carbamate (AMP) (see FIG. 33), or the aminomethyl moeity (AM) (See FIGS. 30, 39 and 40). The release mechanism of this latter utilizes the lability of the hemiaminal functionality, which readily undergoes 1,2-elimination to release the desired alcohol.

For thiol-containing drugs, suitable self-immolative spacers include, but are not limited to, the aminomethyl moeity (AM) (See FIG. 35). The release mechanism of this latter utilizes the lability of the thiohemiaminal functionality, which readily undergoes 1,2-elimination to release the desired thiol.

In some embodiments, the enzymatically cleavable linker is a R-glucuronic acid-based linker. Facile release of the payload may be realized through cleavage of the R-glucuronide glycosidic bond by the lysosomal enzyme R-glucuronidase. This enzyme is present abundantly within lysosomes and is overexpressed in some tumor types, while the enzyme activity outside cells is low. R-Glucuronic acid-based linkers may be used to circumvent the tendency of an antibody-payload conjugate to undergo aggregation due to the hydrophilic nature of R-glucuronides (FIG. 38).

As mentioned above, in a particular embodiment, the invention relates to the peptide linker according to the invention, wherein the chemical linker is or comprises a self-immolative linker.

It is preferred herein that the payloads are attached to the peptide linker via a self-immolative linker to facilitate release of the unmodified drug. Even more preferably, the self-immolative linker is coupled to a peptide sequence that is efficiently cleaved by a protease or a peptidase. The cleavable peptide may be defined as part of the peptide linker or as part of the chemical linker that connects the peptide linker with the payload(s).

The self-immolative linker may be any self-immolative linker known in the art. However, it is preferred that the self-immolative linker comprises a p-aminobenzyl alcohol moiety or a 2,4-bis(hydroxymethyl)aniline moiety.

That is, in a particular embodiment, the invention relates to the peptide linker according to the invention, wherein the self-immolative linker comprises a p-aminobenzyl alcohol moiety or a 2,4-bis(hydroxymethyl)aniline moiety.

Self-immolative linkers comprising a p-aminobenzyl alcohol moiety may be used for coupling payloads to the C-terminus of a peptide. That is, the amino group of the p-aminobenzyl alcohol moiety may be coupled to the C-terminal carboxyl group of the peptide linker via an amide bond (see for example FIG. 3). Alternatively, or in addition, the amino group of the p-aminobenzyl alcohol moiety may be coupled to a carboxyl group in the side chain of an aspartate or glutamate residue in the peptide linker via an amide bond (see for example FIG. 19 or 22)

The payload may be coupled to the hydroxyl group of the p-aminobenzyl alcohol moiety via a carbamate. In certain embodiments, the C-terminal amino acid of the peptide linker to which the p-aminobenzyl alcohol moiety may be coupled may be comprised in a motif that is efficiently cleaved by a peptidase, such as, without limitation, the sequence motif valine-citrulline.

It is to be understood that the peptide linker according to the invention may comprise more than one p-aminobenzyl alcohol moiety. For example, a peptide linker according to the invention may comprise two peptide moieties, wherein the two peptide moieties are linked to each other via their N-terminal ends. In such embodiments, the peptide linker has two C-terminal ends and both C-terminal ends may be conjugated to a payload via a p-aminobenzyl alcohol moiety. An exemplary linker having a payload attached to both termini (to the C-terminus via a p-aminobenzyl alcohol moiety and to the N-terminus via a second peptide moiety and a p-aminobenzyl alcohol moiety) is shown in FIG. 3.

A p-aminobenzyl alcohol moiety may also be used for coupling a payload to an amino acid side chain. For example, a payload may be coupled to the carboxyl group in the side chain of a glutamate or aspartate residue via a p-aminobenzyl alcohol moiety. The p-aminobenzyl alcohol moiety may be coupled to the carboxyl group in the side chain of a glutamate or aspartate residue either directly or via one or more amino acid residues as shown in FIGS. 19, 22, 30, 39 and 40. In certain embodiment, the p-aminobenzyl alcohol moiety may be coupled to the carboxyl group in the side chain of a glutamate or aspartate residue via the valine-citrulline or alanine-alanine sequences.

In certain embodiments, an amine comprising payload may be coupled to a carboxyl group in the peptide linker by two or more aminobenzyl alcohol moieties (see FIG. 3)

Self-immolative linkers comprising a 2,4-bis(hydroxymethyl)aniline moiety may be used for coupling two payloads to a single functional group comprised in a peptide linker. That is, a 2,4-bis(hydroxymethyl)aniline moiety may be coupled to a carboxyl group comprised in a peptide linker via its amino group. Payloads may then be coupled to each of the hydroxyl groups via a carbamate. An exemplary peptide linker wherein two payloads are coupled to the C-terminal end of the peptide linker via a 2,4-bis(hydroxymethyl)aniline moiety is shown FIG. 1.

By using linkers comprising 2,4-bis(hydroxymethyl)aniline moieties, peptide linkers comprising more than two payloads may be obtained. For example, a linker comprising four payloads may be obtained by coupling two payloads to the N-terminal end of a peptide linker via a 2,4-bis(hydroxymethyl)aniline moiety (indirectly via a second peptide moiety) and two more payloads to the C-terminal end of a peptide linker via another 2,4-bis(hydroxymethyl)aniline moiety (see FIG. 20). Similarly, peptide linkers comprising three payloads may be obtained by coupling two payloads to the peptide linker via a 2,4-bis(hydroxymethyl)aniline moiety and a third payload via a p-aminobenzyl alcohol moiety (see FIG. 19).

In a particular embodiment, the invention relates to the peptide linker according to the invention, wherein the hydroxyl group comprised in the p-aminobenzyl alcohol moiety forms a carbamate with a payload.

As mentioned above, a payload may be attached to the p-aminobenzyl alcohol moiety via a carbamate. That is, the payload preferably comprises a free amine group that is suitable to undergo formation of a carbamate. The skilled person is aware of methods to form a carbamate between a p-aminobenzyl alcohol moiety and an amine-comprising payload.

In a particular embodiment, the invention relates to the peptide linker according to the invention, wherein the hydroxyl group comprised in the p-aminobenzyl alcohol moiety forms a carbonate with a payload.

A payload may be attached to the p-aminobenzyl alcohol moiety via a carbonate. That is, the payload preferably comprises a free hydroxyl group that is suitable to undergo formation of a carbonate (see FIG. 34). The skilled person is aware of methods to form a carbonate between a p-aminobenzyl alcohol moiety and a hydroxyl-comprising payload.

In a particular embodiment, the invention relates to the peptide linker according to the invention, wherein each of the hydroxyl groups comprised in the 2,4-bis(hydroxymethyl)aniline moiety forms a carbamate with a payload.

That is, the 2,4-bis(hydroxymethyl)aniline moiety comprised in the peptide linker according to the invention may form two carbamates with two individual amine comprising payloads.

In a particular embodiment, the invention relates to the peptide linker according to the invention, wherein the p-aminobenzyl moiety forms a quaternary ammonium with a payload.

As mentioned above, a payload may be attached to the p-aminobenzyl via a quaternary ammonium. That is, the payload preferably comprises a tertiary- or a heteroaryl-amine that is suitable to undergo formation of a quaternary ammonium. The skilled person is aware of methods to form a quaternary ammonium between a p-aminobenzyl moiety and a tertiary- or a heteroaryl-amine-comprising payload.

In a particular embodiment, the invention relates to the peptide linker according to the invention, wherein the self-immolative linker comprises an ethylenediamine carbamate (EDA) moiety.

That is, payloads may be coupled to the peptide linker according to the invention via an ethylenediamine carbamate (EDA) moiety. An EDA moiety may be coupled directly to the C-terminus of a peptide or to an aspartate or glutamate sidechain via an amide bond. EDA moieties preferably undergo carbamate formation with payloads comprising a hydroxyl group. Examples of linker comprising an EDA moiety are shown in FIGS. 22 and 31. An EDA moiety can also be used to connect an amplifier linked to two payloads as shown in FIG. 28.

In a particular embodiment, the invention relates to the peptide linker according to the invention, wherein the self-immolative linker comprises an (aminomethyl)pyrrolidine-based carbamate (AMP) moiety.

That is, payloads may be coupled to the peptide linker according to the invention via an (aminomethyl)pyrrolidine-based carbamate (AMP) moiety. An AMP moiety may be coupled directly to the C-terminus of a peptide or to an aspartate or glutamate sidechain via an amide bond. AMP moieties preferably undergo carbamate formation with payloads comprising a hydroxyl group. Example of linker comprising an AMP moiety is shown in FIG. 33.

In a particular embodiment, the invention relates to the peptide linker according to the invention, wherein the self-immolative linker comprises an aminomethyl (AM) moiety.

That is, payloads may be coupled to the peptide linker according to the invention via an aminomethyl (AM) moiety. An AM moiety may be coupled directly to the C-terminus of a peptide or to an aspartate or glutamate sidechain via an amide bond. AM moieties are preferably used to link payloads comprising a hydroxyl group, thereby forming a hemiaminal. Examples of linker comprising an AM moiety are shown in FIGS. 30, 39 and 40. However, AM moieties can also be used to link payloads comprising a thiol group, thereby forming a thiohemiaminal. Example of linker comprising an AM moiety with a thiol-containing payload is shown in FIG. 35.

In a particular embodiment, the invention relates to the peptide linker according to the invention, wherein at least one payload is attached to a side chain of a glutamate, aspartate, tryptophan, cysteine, lysine, tyrosine, serine, or threonine residue comprised in the peptide linker.

As mentioned above, one or more payloads may be coupled to an amino acid side chain comprised in the peptide linker. The skilled person is aware of chemical linkers that are suitable for coupling a payload to an amino acid side chain, i.e., the carboxyl group in the side chain of a glutamate or aspartate residue, the thiol group in the side chain of a cysteine residue, the amino group in the side chain of a lysine residue or the hydroxy group in the side chain of a tyrosine, serine, or threonine residue. Example of linker comprising a payload on an amino acid side chain are shown in FIGS. 19, 22, 29, 30, 35, 36, 37, 39 and 40.

In a particular embodiment, the invention relates to the peptide linker according to the invention, wherein the peptide linker comprises two peptide moieties, and wherein the two peptide moieties are connected via their N-terminal amino acid residues with a dicarboxylic acid linker (HO₂C—R—CO₂H).

Certain linkers falling within the scope of the present invention comprise two peptide moieties, wherein the two peptide moieties are linked via their N-terminal amino acid residues (see FIG. 3 for an example). The N-terminal amino acids of the two peptide moieties may be linked via a dicarboxylic acid, wherein each carboxylic acid group comprised in the dicarboxylic acid forms an amide bond with an N-terminal amino group of a peptide moiety.

Any dicarboxylic acid may be used to link two peptide moieties via their N-terminal amino acid residues. In certain embodiments, the dicarboxylic acid may be an aliphatic dicarboxylic acid. That is, the dicarboxylic acid may be ethanedioic acid, propanedioic acid, butanedioic acid, pentanedioic acid, hexanedioic acid, heptanedioic acid, octanedioic acid, nonanedioic acid or decanedioic acid. In certain embodiments, two peptide moieties are linked via their N-terminal amino acids with a butanedioic acid molecule (see FIGS. 3, 14, 20, 21, 27 and 34). In certain embodiments, two peptide moieties are linked via their N-terminal amino acids with a pentanedioic acid molecule (see FIG. 26). Aliphatic dicarboxylic acids may comprise substituted or unsubstituted alkyl or alkenyl chains.

In certain embodiments, the dicarboxylic acid may be an aromatic dicarboxylic acid. Aromatic dicarboxylic acids include, without limitation, phthalic acid, isophthalic acid, or terephthalic acid.

It is to be understood that linking two peptide moieties via their N-terminal amino acids results in a peptide construct comprising no free N-terminal amino group that may be suitable for conjugation to a glutamine residue comprised in an antibody. Thus, peptide linkers comprising two N-terminally linked peptide moieties preferably comprise a lysine residue, a lysine mimetic or a lysine derivative to enable conjugation of the peptide linker to a glutamine moiety comprised in an antibody.

In certain embodiments, the first peptide moiety comprised in the peptide linker may comprise any of the amino acid sequences set forth in SEQ ID NO:1-8, 11-14, 16-29 or 82-93. The second peptide moiety may have any amino acid sequence. In certain embodiments, the second peptide moiety may have a length of 2-100, preferably 2-50, more preferably 2-25, even more preferably 2-10, most preferably 2-5 amino acid residues. In certain embodiments, the second peptide moiety may be a dipeptide or a tripeptide. However, it is to be noted that the second peptide moiety may also be a single amino acid or a longer peptide. To enable efficient release of the payload, the second peptide moiety preferably comprises a peptide sequence that is efficiently cleaved by a peptidase. In certain embodiments, the second peptide moiety may have the sequence Asn, Ala, Ala-Ala, Ala-Asn, Val-Ala, Val-Cit, Ala-Arg, Arg-Ala, Ala-Ala-Arg (SEQ ID NO:34), Ala-Arg-Ala (SEQ ID NO:35), Ala-Ala-Asn (SEQ ID NO:36).

That is, in certain embodiments, the peptide linker may have the structure:

[payload1]-[peptide1]-[dicarboxylic acid]-[peptide2]-[payload2]; wherein
[payload 1] and [payload 2] are payloads,
[peptide 1] is a first peptide moiety,
[peptide 2] is a second peptide moiety, and
[dicarboxylic acid] is a dicarboxylic acid;
wherein at least one of the peptide moieties 1 and/or 2 comprises a free amine,
wherein the N-terminal end of peptide 1 and the N-terminal end of peptide 2 are connected via the dicarboxylic acid,
wherein payload 1 is attached to the C-terminal end of peptide 1, preferably via a chemical linker, and
wherein payload 2 is attached to the C-terminal end of peptide 2, preferably via a chemical linker.

Preferably, the peptide moiety comprising a free amine group is a peptide moiety comprising a lysine residue, a lysine mimetic or a lysine derivative, as defined elsewhere herein or a peptide linker comprising any one of the amino acid sequences set forth in SEQ ID NO:1-8, 11-14, 16-29 or 82-93.

In certain embodiments, the peptide linker may comprise a first peptide moiety comprising a sequence set forth in SEQ ID NO:1-8, 11-14, 16-29 or 82-93 and a second moiety comprising the sequence Ala-Ala, wherein the first and second peptide moiety are linked via their N-terminal amino acids with a butanedioic acid molecule.

In certain embodiments, the peptide linker may comprise a first peptide moiety comprising the sequence RKAA and a second moiety comprising the sequence Ala-Ala, wherein the first and second peptide moiety are linked via their N-terminal amino acids with a butanedioic acid molecule.

Instead of coupling two peptide moieties via their N-terminal amino acid residues, a second peptide moiety may also be coupled to an amino acid side chain of a first peptide moiety. That is, the first peptide moiety comprised in the peptide linker may comprise any of the amino acid sequences set forth in SEQ ID NO:6, 11-12, 20-21, 23, 26-29 or 82-93. The second peptide moiety, that is the one positioned on the amino acid side chain, may have any amino acid sequence. In certain embodiments, the second peptide moiety may be a dipeptide or a tripeptide. However, it is to be noted that the second peptide moiety may also be a single amino acid or a longer peptide. To enable efficient release of the payload, the second peptide moiety preferably comprises a peptide sequence that is efficiently cleaved by a peptidase. In certain embodiments, the second peptide moiety may have the sequence Asn, Ala, Ala-Ala, Ala-Asn, Val-Ala, Val-Cit, Ala-Arg, Arg-Ala, Ala-Ala-Arg (SEQ ID NO:34), Ala-Arg-Ala (SEQ ID NO:35), Ala-Ala-Asn (SEQ ID NO:36).

The peptide linker according to the invention comprises two or more payloads. The peptide linkers comprising the two or more payloads are preferably obtained by chemical synthesis.

The skilled person is aware of methods for coupling a payload to an amino acid-based linker by chemical synthesis. For example, an amine-comprising payload (for e.g. auristatin analogs), or a thiol-comprising payload (for e.g. maytansine analogs), or a hydroxyl-containing payload (for e.g. SN-38 analogs) may be attached to the C-terminus of an amino acid-based linker by chemical synthesis. However, the skilled person is aware of further reactions and reactive groups that may be utilized for coupling a payload to the N-terminus, C-terminus or the side chain of an amino acid or amino acid derivative by chemical synthesis. Typical reactions that may be used for coupling a payload to an amino acid-based linker by chemical synthesis include, without limitation: peptide coupling, activated ester coupling (NHS ester, PFP ester), click reaction (CuAAC, SPAAC), Michael addition (thiol maleimide conjugation).

The coupling of payloads to peptides has been extensively described in the prior art, for example by Costoplus et al. (Peptide-Cleavable Self-immolative Maytansinoid Antibody-Drug Conjugates Designed To Provide Improved Bystander Killing. ACS Med Chem Lett. 2019 Sep. 27; 10(10):1393-1399), Sonzini et al. (Improved Physical Stability of an Antibody-Drug Conjugate Using Host-Guest Chemistry. Bioconjug Chem. 2020 Jan. 15; 31(1):123-129), Bodero et al. (Synthesis and biological evaluation of RGD and isoDGR peptidomimetic-a-amanitin conjugates for tumor-targeting. Beilstein J. Org. Chem. 2018, 14, 407-415), Nunes et al. (Use of a next generation maleimide in combination with THIOMAB™ antibody technology delivers a highly stable, potent and near homogeneous THIOMAB™ antibody-drug conjugate (TDC). RSC Adv., 2017, 7, 24828-24832), Doronina et al. (Enhanced activity of monomethylauristatin F through monoclonal antibody delivery: effects of linker technology on efficacy and toxicity. Bioconjug Chem. 2006 January-February; 17(1):114-24), Nakada et al. (Novel antibody drug conjugates containing exatecan derivative-based cytotoxic payloads. Bioorg Med Chem Lett. 2016 Mar. 15; 26(6):1542-1545) and Dickgiesser et al. (Site-Specific Conjugation of Native Antibodies Using Engineered Microbial Transglutaminases. Bioconjug Chem. 2020 Mar. 12. doi: 10.1021/acs.bioconjchem.0c00061).

It is to be understood that the payload may be coupled to the N-terminal and/or to the C-terminal end of a peptide-based or a peptide-comprising linker according to the invention. In certain embodiments, a payload may be coupled directly to the N-terminal amino group or the C-terminal carboxyl group of a peptide or an amino acid residue.

The skilled person is aware of reactive groups that are suitable for coupling a payload to an amino acid residue. For example, an amine-comprising payload may be coupled to the C-terminal carboxyl group of an amino acid residue via an amide bond. Alternatively, a payload comprising a thiol group or and hydroxyl group may be coupled to the C-terminal carboxyl group of an amino acid via a thioester or an ester bond (see FIG. 34), respectively. A payload comprising a carboxylic acid group may be coupled to the N-terminal amino group of an amino acid residue via an amide bond (see FIG. 32).

In certain embodiments, a payload may be coupled indirectly to the N- and/or C-terminal end of a peptide or amino acid residue comprised in the linker according to the invention. The skilled person is aware of linker molecules that may be used to couple a payload to the N-terminal amino group or the C-terminal carboxyl group of an amino acid residue comprised in the linker according to the invention.

In certain embodiments, a payload comprising a hydroxyl group may be coupled to the N-terminus of an amino acid residue via a linker molecule. For example, payloads comprising a hydroxyl group may be coupled to an N-terminal amino group via a carbamate linker.

In certain embodiments, a payload comprising a thiol group may be coupled to the N-terminus of an amino acid residue via a linker molecule. For example, payloads comprising a thiol group may be coupled to an N-terminal amino group via a thiocarbamate linker. Alternatively, payloads comprising a thiol group may be coupled to an N-terminal amino group via an alkyl linker molecule comprising a carboxyl group and a thiol group. In certain embodiments the alkyl linker molecule may be a 3-mercaptopropionic acid linker molecule, wherein the payload forms a di-sulfur bond with the thiol group comprised in the 3-mercaptopropionic acid linker molecule.

In certain embodiments, a payload comprising an amide group may be coupled to the N-terminus of an amino acid residue via a linker molecule. For example, payloads comprising an amine group may be coupled to an N-terminal amino group via a dicarboxylic acid linker molecule, wherein the each of the carboxylic acid groups comprised in the dicarboxylic acid linker forms an amide bond with the payload and the amino group of the N-terminal amino acid residue. Examples of dicarboxylic acids that may be used as linker molecules in the present invention are, without limitation, succinic acid or pimelic acid.

Alternative linker molecules for indirectly coupling payloads to the N-terminus of an amino acid residue comprised in the peptide linker according to the invention or linker molecules that are suitable for indirectly coupling payloads to the C-terminus of an amino acid residue comprised in the peptide linker according to the invention have been described in the art and are encompassed by the present invention.

The inventors surprisingly found that peptide linkers having payloads attached to the N-terminal end and to the C-terminal end of the peptide linker have stronger anti-tumor activity than linkers having all payloads attached to the C-terminal end of the peptide linker (see Example 11 and Example 12).

Accordingly, in a particular embodiment, the invention relates to the peptide linker according to the invention, wherein one or more payload is attached to the N-terminal end of an amine-comprising peptide linker and wherein one or more payload is attached to the C-terminal end of said amine-comprising peptide linker.

In certain embodiments, one or two payloads may be attached to the N-terminal end of an amine-comprising peptide linker and one or two payloads may be attached to the C-terminal end of said amine-comprising peptide linker. That is, the peptide linker may be a DAR4, DAR6 or DAR8 linker.

In certain embodiments, one payload may be attached to the N-terminal end of an amine-comprising peptide linker and one payload may be attached to the C-terminal end of said amine-comprising peptide linker. In such embodiments, the peptide linker may be a “linear” DAR4 linker.

The amine-comprising peptide linker maybe any one of the lysine-comprising peptide linkers disclosed herein, including peptide linkers comprising a lysine mimetic or a lysine derivative as defined herein.

In certain embodiments, the “linear” DAR4 linker may have the following structure (in N->C direction):

[payload1]-[(Aa)ₘ-(Lys)-(Aa)ₙ]-[payload2]; wherein
[payload 1] and [payload 2] are payloads,
Aa may be any amino acid residue;
m and n may be integers ranging from 0 to 10, preferably 0 to 6, more preferably 0 to 4; and
Lys is a lysine residue, a lysine mimetic or a lysine derivative,
wherein [payload 1] is directly or indirectly attached to an N-terminal end of an (Aa) or (Lys) residue, and
wherein [payload 2] is directly or indirectly attached to a C-terminal end of an (Aa) or (Lys) residue.

In certain embodiments, the “linear” DAR4 linker may have the following structure:

[payload1]-[(Aa)ₘ-(Arg/His)-(Aa)ₙ-(Lys)-(Aa)ₒ]-[payload2]; wherein
[payload 1] and [payload 2] are payloads,
Aa may be any amino acid residue;
m, n and o may be integers ranging from 0 to 10, preferably 0 to 6, more preferably 0 to 4; and
Arg may be an arginine residue, an arginine mimetic or an arginine derivative;
His may be a histidine residue, a histidine mimetic or a histidine derivative
Lys is a lysine residue, a lysine mimetic or a lysine derivative,
wherein [payload 1] is directly or indirectly attached to an N-terminal end of an (Aa) or (Arg/His) residue, and
wherein [payload 2] is directly or indirectly attached to a C-terminal end of an (Aa) or (Lys) residue.

In certain embodiments, the “linear” DAR4 linker may have the following structure:

[payload1]-[(Aa)ₘ-(Lys)-(Aa)ₙ-(Arg/His)-(Aa)ₒ]-[payload2]; wherein
[payload 1] and [payload 2] are payloads,
Aa may be any amino acid residue;
m, n and o may be integers ranging from 0 to 10, preferably 0 to 6, more preferably 0 to 4; and
Arg may be an arginine residue, an arginine mimetic or an arginine derivative;
His may be a histidine residue, a histidine mimetic or a histidine derivative
Lys is a lysine residue, a lysine mimetic or a lysine derivative,
wherein [payload 1] is directly or indirectly attached to an N-terminal end of an (Aa) or (Lys) residue, and
wherein [payload 2] is directly or indirectly attached to a C-terminal end of an (Aa) or (Arg/His) residue.

It is to be understood that the payloads may be directly or indirectly attached to the N-terminal end and to the C-terminal end of the peptide linker. In one embodiment, a first payload may be directly attached to the N-terminal amino group of the peptide linker and a second payload may be directly attached to the C-terminal carboxyl group of the peptide linker.

It is, however, preferred that the payloads are indirectly attached to the N-terminal end and to the C-terminal end of the peptide linker, for example with any one of the chemical linkers described herein. In particular, a payload may be indirectly attached to the N-terminal end of the peptide linker via a dicarboxylic acid and a second peptide moiety, as described in more detail elsewhere herein. Furthermore, it is preferred that all payloads are attached to the peptide linker or the chemical linker via a self-immolative moiety, such as any one of the self-immolative moiety disclosed herein.

In a particular embodiment, the invention relates to the peptide linker according to the invention, wherein the payload is at least one of:
a toxin;
a cytokine;
a growth factor;
a radionuclide;
a hormone;
an anti-viral agent;
an anti-bacterial agent;
a fluorescent dye;
an immunoregulatory/immunostimulatory agent;
a half-life increasing moiety;
a solubility increasing moiety;
a polymer-toxin conjugate;
a nucleic acid;
a biotin or streptavidin moiety;
a vitamin;
a protein degradation agent (‘PROTAC’);
a ligand of a receptor;
a target binding moiety; and/or
an anti-inflammatory agent.

In certain embodiments, the payload may be a cytokine. The term “cytokine,” as used herein, means any secreted polypeptide that affects the functions of other cells, and that modulates interactions between cells in the immune or inflammatory response. Cytokines include, but are not limited to monokines, lymphokines, and chemokines regardless of which cells produce them. For instance, a monokine is generally referred to as being produced and secreted by a monocyte, however, many other cells produce monokines, such as natural killer cells, fibroblasts, basophils, neutrophils, endothelial cells, brain astrocytes, bone marrow stromal cells, epidermal keratinocytes, and B-lymphocytes. Lymphokines are generally referred to as being produced by lymphocyte cells. Examples of cytokines include, but are not limited to, interleukin-1 (IL-1), interleukin-6 (IL-6), Tumor Necrosis Factor alpha (TNFα), and Tumor Necrosis Factor beta (TNFβ).

In certain embodiments, the payload may be an anti-inflammatory agent. As used herein, the term “anti-inflammatory agent” means those agent classes whose main mode of action and use is in the area of treating inflammation and also any other agent from another therapeutic class that possesses useful anti-inflammatory effects. Such anti-inflammatory agents include, but are not limited to non-steroidal anti-inflammatory drugs (NSAIDs), disease modifying anti-rheumatic drugs (DMARDs), macrolide antibiotics and statins. Preferably, the NSAIDs include, but are not limited to, salicylates (e.g. aspirin), arylpropionic acids (e.g. ibuprofen), anthranilic acids (e.g. mefenamic acid), pyrazoles (e.g. phenylbutazone), cyclic acetic acids (indomethacin) and oxicams (e.g. piroxicam). Preferably, anti-inflammatory agents for use in the methods of the present invention include sulindac, diclofenac, tenoxicam, ketorolac, naproxen, nabumetone, diflunasal, ketoprofen, arlypropionic acids, tenidap, hydroxychloroquine, sulfasalazine, celecoxib, rofecoxib, meloxicam, etoricoxib, valdecoxib, methotrexate, etanercept, infliximab, adalimumab, atorvastatin, fluvastatin, lovastatin, pravastatin, simvastatin, clarithromycin, azithromycin, roxithromycin, erythromycin, ibuprofen, dexibuprofen, flurbiprofen, fenoprofen, fenbufen, benoxaprofen, dexketoprofen, tolfenamic acid, nimesulide and oxaprozin.

In certain embodiments, the anti-inflammatory agent may be an anti-inflammatory cytokine, which, when conjugated to a target specific antibody, can ameliorate inflammations caused, e.g., by autoimmune diseases. Cytokines with anti-inflammatory activities may be, without limitation, IL-IRA, IL-4, IL-6, IL-10, IL-11, IL-13 or TGF-β.

In certain embodiments, the payload may be a growth factor. The term “growth factor” as used herein refers to a naturally occurring substance capable of stimulating cellular growth, proliferation, cellular differentiation, and/or cellular maturation. Growth factors exist in the form of either proteins or steroid hormones. Growth factors are important for regulating a variety of cellular processes. Growth factors typically act as signaling molecules between cells. However, their ability to promote cellular growth, proliferation, cellular differentiation, and cellular maturation varies between growth factors. A non-limiting list of examples of growth factors includes: basic fibroblast growth factor, adrenomedullin, angiopoietin, autocrine motility factor, bone morphogenetic proteins, brain-derived neurotrophic factor, epidermal growth factor, epithelial growth factor, fibroblast growth factor, glial cell line-derived neurotrophic factor, granulocyte colony-stimulating factor, granulocyte macrophage colony-stimulating factor, growth differentiation factor-9, hepatocyte growth factor, hepatoma-derived growth factor, insulin growth factor, insulin-like growth factor, migration-stimulating factor, myostatin, nerve growth factor, and other neurotrophins, platelet-derived growth factor, transforming growth factor alpha, transforming growth factor beta, tumor-necrosis-factor-alpha, vascular endothelial growth factor, placental growth factor, fetal bovine somatotrophin, and cytokines (e.g. IL-1-cofactor for IL-3 and IL-6, IL-2-t-cell growth factor, IL-3, IL-4, IL-5, IL-6, and IL-7).

In certain embodiments, the payload may be a hormone. The term “hormone”, as used herein, refers to a chemical released by a cell or a gland in one part of the body that sends out messages that affect cells in other parts of the organism. Examples of hormones that are useful in the present invention are, without limitation, melatonin (MT), serotonin (5-HT), thyroxine (T4), triiodothyronine (T3), epinephrine or adrenaline (EPI), norepinephrine or noradrenaline (NRE), dopamine (DPM or DA), antimullerian hormone or mullerian inhibiting hormone (AMH), adiponectin (Acrp30), adrenocorticotropic hormone or corticotrophin (ACTH), angiotensinogen and angiotensin (AGT), antidiuretic hormone or vasopressin (ADH), atrial natriuretic peptide or atriopeptin (ANP), calcitonin (CT), cholecystokinin (CCK), corticotrophin-releasing hormone (CRH), erythropoietin (EPO), follicle-stimulating hormone (FSH), gastrin (GRP), ghrelin, glucagon (GCG), gonadotrophin-releasing hormone (GnRH), growth hormone-releasing hormone (GHRH), human chorionic gonadotrophin (hCG), human placental lactogen (HPL), growth hormone (GH or hGH), inhibin, insulin (INS), insulin-like growth factor or somatomedin (IGF), leptin (LEP), luteinizing hormone (LH), melanocyte stimulating hormone (MSH or α-MSH), orexin, oxytocin (OXT), parathyroid hormone (PTH), prolactin (PRL), relaxin (RLN), secretin (SCT), somatostatin (SRIF), thrombopoietin (TPO), thyroid-stimulating hormone or thyrotropin (TSH), thyrotropin-releasing hormone (TRH), cortisol, aldosterone, testosterone, dehydroepiandrosterone (DHEA), androstenedione, dihydrotestosterone (DHT), estrone, estriol (E3), progesterone, calcitriol, calcidiol, prostaglandins (PG), leukotrienes (LT), prostacyclin (PGI2), thromboxane (TXA2), prolactin releasing hormone (PRH), lipotropin (PRH), brain natriuretic peptide (BNP), neuropeptide Y (NPY), histamine, endothelin, pancreatic polypeptide, renin and enkephalin. In a particular embodiment, the hormone is cortisol.

In certain embodiments, the payload may be an antiviral agent. The term “antiviral agent” as used herein means an agent (compound or biological) that is effective to inhibit the formation and/or replication of a virus in a mammal. This includes agents that interfere with either host or viral mechanisms necessary for the formation and/or replication of a virus in a mammal. Antiviral agents include, for example, ribavirin, amantadine, VX-497 (merimepodib, Vertex Pharmaceuticals), VX—498 (Vertex Pharmaceuticals), Levovirin, Viramidine, Ceplene (maxamine), XTL-001 and XTL-002 (XTL Biopharmaceuticals).

In certain embodiments, the payload may be an antibacterial agent. The term “antibacterial agent” as used herein refers to any substance, compound, a combination of substances, or a combination of compounds capable of: (i) inhibiting, reducing or preventing growth of bacteria; (ii) inhibiting or reducing ability of a bacteria to produce infection in a subject; or (iii) inhibiting or reducing ability of bacteria to multiply or remain infective in the environment. The term “antibacterial agent” also refers to compounds capable of decreasing infectivity or virulence of bacteria.

Suitable antibiotics that may be used as a payload in the present invention include, but are not limited to: a macrolide, a penicillin, a cephalosporin, a quinolone, a fluoroquinolone, a sulphonamide, a tetracycline, a monobactam, a carbapenem, an aminoglycoside, a rifamycin, a beta-lactam, an ansamycin, an oxazolidinone, a streptogramin, a glycopeptide, a polypeptide, and an arsphenamine, or a pharmaceutically acceptable salt thereof, more preferably wherein said antibiotic is selected from erythromycin, azithromycin, clarithromycin, dirithromycin, clindamycin, doxycycline, minocycline, tigecycline, trimethoprim, pyocyanine, vancomycin, streptomycin, dihydrostreptomycin, amikacin, apramycin, arbekacin, astromicin, bekanamycin, dibekacin, framycetin, gentamicin, hygromycin, isepamicin, kanamycin, neomycin, netilmicin, paromomycin, rhodostreptomycin, ribostamycin, sisomycin, spectinomycin, tobramycin, verdamicin, polymixin, glycylcycline, carbapenem, carbacephem, chloramphenicol, clindamycin, lincomycin, daptomycin, novobiocin, clindamycin, ethambutol, fosfomycine, fusidic acid, furazolidone, isoniazid, linezolide, metronidazole, mupirocin, nitrofurantoin, platensimycine, pyrazinamide, quinupristine, benzalkonium, dalfopristine, rifampine, rifampicin, rifabutin, rifaximin, rifalog, tinidazole, viomycin, and capreomycin, or a pharmaceutically acceptable salt thereof.

In certain embodiments, the peptide linker according to the invention comprises rifalog as a payload (see FIG. 32).

In certain embodiments, the payload may be an immunoregulatory agent. The term “immunoregulatory agent” as used herein for combination therapy refers to substances that act to suppress, mask, or enhance the immune system of the host. Examples of immunomodulatory agents include, but are not limited to, proteinaceous agents such as cytokines, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules, iRNA and triple helices), small molecules, organic compounds, and inorganic compounds. In particular, immunomodulatory agents include, but are not limited to, methothrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunomide), T cell receptor modulators, and cytokine receptor modulators.

In certain embodiments, the immunoregulatory agent may be an immunostimulatory agent. The term “immunostimulatory agent” as used herein preferably refers to any substance or substance that can trigger an immune response (e.g., an immune response against a particular pathogen). Immune cell activating compounds include Toll-like receptor (TLR) agonists. Such agonists include pathogen associated molecular patterns (PAMPs), e.g., an infection-mimicking composition such as a bacterially-derived immunomodulator (a.k.a., danger signal) and damage associated molecular pattern (DAMPs), e.g. a composition mimicking a stressed or damaged cell. TLR agonists include nucleic acid or lipid compositions (e.g., monophosphoryl lipid A (MPLA)). In one example, the TLR agonist comprises a TLR9 agonist such as a cytosine-guanosine oligonucleotide (CpG-ODN), a poly(ethylenimine) (PEI)-condensed oligonucleotide (ODN) such as PEI-CpG-ODN, or double stranded deoxyribonucleic acid (DNA). In another example, the TLR agonist comprises a TLR3 agonist such as polyinosine-polycytidylic acid (poly (I:C)), PEI-poly (I:C), polyadenylic-polyuridylic acid (poly (A:U)), PEI-poly (A:U), or double stranded ribonucleic acid (RNA). Other exemplary vaccine immunostimulatory compounds include STING agonists (for ex: STING agonist-3, extracted from patent WO2017175147A1, example 10), lipopolysaccharide (LPS), chemokines/cytokines, fungal beta-glucans (such as lentinan), imiquimod, CRX-527, and OM-174.

In certain embodiments, the immunostimulatory agent may be a toll-like receptor (TLR) 7/8 agonist, such as, without limitation Imiquimod, Resiquimod, 852-A, Vesatolimod, AZD8848, Motolimod or Selgantolimod.

In certain embodiments, the peptide linker of the invention may comprise two different immunostimulatory agents. For example, the peptide linker of the invention may comprise STING agonist 3 and Resiquimod (see FIG. 34).

In certain embodiments, the payload may be a half-life increasing moiety or a solubility increasing moiety. Half-life increasing moieties are, for example, PEG-moieties (polyethyleneglycol moieties; PEGylation), other polymer moieties, PAS moieties (oligopeptides comprising Proline, Alanine and Serine; PASylation), or Serum albumin binders. Solubility increasing moieties are, for example PEG-moieties (PEGylation) or PAS moieties (PASylation).

In certain embodiments, the payload may be a polymer-toxin conjugate. Polymer-toxin conjugates are polymers that are capable of carrying one or many payload molecules. Examples include Fleximer polymer-toxin developed by Mersana therapeutics, PSAR polymer-toxin developed by Mablink, XTEN polymer-toxin developed by Amunix. or A polymer-toxin conjugate may comprise any of the toxins disclosed herein.

In certain embodiments, the payload may be a nucleotide. One example of a nucleic acid payload is MCT-485, which is a very small non-coding double stranded RNA which has oncolytic and immune activating properties, developed by MultiCell Technologies, Inc.

In certain embodiments, the payload may be a fluorescent dye. The term “fluorescent dye” as used herein refers to a dye that absorbs light at a first wavelength and emits at second wavelength that is longer than the first wavelength. In certain embodiment, the fluorescent dye is a near-infrared fluorescent dye, which emits light at a wavelength between 650 and 900 nm. In this region, tissue autofluorescence is lower, and less fluorescence extinction enhances deep tissue penetration with minimal background interference. Accordingly, near-infrared fluorescent imaging may be used to make tissues that are bound by the antibody-payload conjugate of the invention visible during surgery. “Near-infrared fluorescent dyes” are known in the art and commercially available. In certain embodiments, the near-infrared fluorescent dye may be IRDye 800CW, Cy7, Cy7.5, NIR CF750/770/790, DyLight 800 or Alexa Fluor 750.

In certain embodiments, the payload may comprise a radionuclide. The term “radionuclide”, as used herein, relates to medically useful radionuclides, including, for example, positively charged ions of radiometals such as Y, In, Tb, Ac, Cu, Lu, Tc, Re, Co, Fe and the like, such as 90Y, 111In, 67Cu, 77Lu, 99Tc, 161Tb, 225Ac and the like. The radionuclide may be comprised in a chelating agent such as DOTA or NODA-GA. Further, the radionuclide may be a therapeutic radionuclide or a radionuclide that can be used as contrast agent in imaging techniques as discussed below. Radionuclides or molecules comprising radionuclides are known in the art and commercially available.

In certain embodiments, the payload may be a ligand of a receptor or substrate of a receptor. In particular, the payload may be a ligand or substrate of a receptor that is known to be strongly expressed in cancer cells. That is, coupling the ligands or substrates of such receptors to an antibody via the peptide linker of the invention may improve the specificity of the antibody-payload conjugate and may further improve internalization of the antibody-payload conjugate into target cells, such as cancer cells. For example, the payload may be folate to improve the targeting of cancer cells that overexpress the folate receptor FRα. However, the payload may also be a derivative or analog of folate that binds to FRα with high affinity. Further, the payload may be any other ligand or substrate of FRα, in particular a ligand or substrate that binds to FRα with high affinity.

In other embodiments, the payload may be a ligand or substrate of a biotin receptor. That is, the payload may be biotin, a biotin analog or derivative, or any other molecule that binds to a biotin receptor with high affinity.

In other embodiments, the payload may be a ligand or substrate of an epidermal growth factor receptor (EGFR). That is, the payload may be epidermal growth factor (EGF) or any derivative, analog or fragment thereof that binds to EGFR with high affinity. Further the payload may be any molecule that binds to EGFR with high affinity.

Other examples of peptides/small molecule ligands which could be used as payloads to target receptors known to be strongly expressed in cancer cells include, but are not limited to, tumor-homing peptides: RGD peptides and their derivatives (iRGD, cilengitide, SFITGv6, CNGRC etc.), extracellular matrix-homing peptides (DAG, ZD2, CSG, PlGF-2, BT1718), tumor associated macrophages-targeting agents (RP-182, M2pep, mUNO), EGFR targeting peptide (GE11), Angiopep-2, peptides targeting aberrant cellular signaling pathways (LP4, NBD, H1), PSMA binders (urea-based or phosphoramidate-based binders).

Both a “ligand” and a “substrate” are defined herein as molecules that bind to a receptor with a certain affinity. However, it is to be understood that a “ligand” is typically a small molecule, while a “substrate” is typically a macromolecule, such as a peptide or protein. It is to be understood that the ligand or substrate comprised in the peptide linker according to the invention may be a naturally occurring ligand or substrate, a derivative of a naturally occurring ligand or substrate or a chemically modified version of a naturally occurring ligand or substrate.

In certain embodiments, the payload may be a vitamin. The vitamin may be selected from the group consisting of folates, including folic acid, folacin, and vitamin B9. The vitamin may be selected from the group consisting of biotin, vitamin B7.

In a particular embodiment, the invention relates to the peptide linker according to the invention, wherein the toxin is at least one selected from the group consisting of:
a pyrrolobenzodiazepine (e.g., PBD);
an auristatin (e.g., MMAE, MMAF);
a maytansinoid (e.g., maytansine, DM1, DM4, DM21);
a duocarmycin;
a nicotinamide phosphoribosyltransferase (NAMPT) inhibitor;
a tubulysin;
an enediyne (e.g., calicheamicin);
an anthracycline derivative (PNU) (e.g., doxorubicin);
a pyrrole-based kinesin spindle protein (KSP) inhibitor;
a cryptophycin;
a drug efflux pump inhibitor;
a sandramycin;
a thymidylate synthase inhibitor;
an amanitin (e.g., a-amanitin); and
a camptothecin (e.g., exatecans, deruxtecans).

That is, the peptide linkers of the invention preferably comprise a toxin payload. The term “toxin” as used herein relates to any compound that is poisonous to a cell or organism. Preferably, the toxin is produced by a cell or an organism. However, the toxin may also be a chemical derivative or analog of a toxin that is produced by a cell or an organism. Toxins can be, without limitation, small molecules, peptides, or proteins. Specific examples are neurotoxins, necrotoxins, hemotoxins and cytotoxins. In certain embodiments, the toxin is a toxin that is used in the treatment of neoplastic diseases. That is, the toxin may be conjugated to an antibody with the method of the invention and delivered to or into a malignant cell due to the target specificity of the antibody.

In certain embodiments, the toxin may be an auristatin. As used herein, the term “auristatin” refers to a family of anti-mitotic agents. Auristatin derivatives are also included within the definition of the term “auristatin”. Examples of auristatin include, but are not limited to, synthetic analogues of auristatin E (AE), monomethyl auristatin E (MMAE), monomethyl auristatin F (MMAF) and dolastatin.

In certain embodiments, the toxin may be a maytansinoid. In the context of the present invention, the term “maytansinoid” refers to a class of highly cytotoxic drugs originally isolated from the African shrub Maytenus ovatus and further maytansinol (Maytansinol) and C-3 ester of natural maytansinol (U.S. Pat. No. 4,151,042); C-3 ester analog of synthetic maytansinol (Kupchan et al., J. Med. Chem. 21: 31-37, 1978; Higashide et al., Nature 270: 721-722, 1977; Kawai et al., Chem. Farm. Bull. 32: 3441-3451; and U.S. Pat. No. 5,416,064); C-3 esters of simple carboxylic acids (U.S. Pat. Nos. 4,248,870; 4,265,814; 4,308,268; 4,308,269; 4,309,428; 4,317,821; 4,322,348; and 4,331,598); and C-3 esters with derivatives of N-methyl-L-alanine (U.S. Pat. Nos. 4,137,230; 4,260,608; and Kawai et al., Chem. Pharm Bull. 12: 3441, 1984). Exemplary maytansinoids that may be used in the method of the invention or that may be comprised in the antibody-payload conjugate of the invention are maytansine, DM1, DM3, DM4 and/or DM21.

In certain embodiments, the toxin may be a duocarmycin. Suitable duocarmycins may be e.g. duocarmycin A, duocarmycin BI, duocarmycin B2, duocarmycin CI, duocarmycin C2, duocarmycin D, duocarmycin SA, duocarmycin MA, and CC-1065. The term “duocarmycin” should be understood as referring also to synthetic analogs of duocarmycins, such as adozelesin, bizelesin, carzelesin, KW-2189 and CBI-TMI.

In certain embodiments, the toxin may be a NAMPT inhibitor. As used herein, the terms “NAMPT inhibitor” and “nicotinamide phosphoribosyl transferase inhibitor” refer to an inhibitor that reduces the activity of NAMPT. The term “NAMPT inhibitor” may also include prodrugs of a NAMPT inhibitor. Examples of NAMPT inhibitors include, without limitation, FK866 (also referred to as APO866), GPP 78 hydrochloride, ST 118804, STF31, pyridyl cyanoguanidine (also referred to as CH-828), GMX-1778, and P7C3. Additional NAMPT inhibitors are known in the art and may be suitable for use in the compositions and methods described herein. See, e.g., PCT Publication WO 2015/054060, U.S. Pat. Nos. 8,211,912, and 9,676,721, which are incorporated by reference herein in their entireties. In some embodiments, the NAMPT inhibitor is FK866. In some embodiments, the NAMPT inhibitor is GMX-1778.

In certain embodiments, the toxin may be a tubulysin. Tubulysins are cytotoxic peptides, which include 9 members (A-I). Tubulysin A has potential application as an anticancer agent. It arrests cells in the G2/M phase. Tubulysin A inhibits polymerization more efficiently than vinblastine and induces depolymerization of isolated microtubules. Tubulysin A has potent cytostatic effects on various tumor cell lines with IC50 in the picomolar range. Other tubulysins that may be used in the method of the invention may be tubulysin E.

In certain embodiments, the toxin may be an enediyne. The term “enediyne,” as used herein, refers to a class of bacterial natural products characterized by either nine- and ten-membered rings containing two triple bonds separated by a double bond (see, e.g., K. C. Nicolaou; A. L. Smith; E. W. Yue (1993). “Chemistry and biology of natural and designed enediynes”. PNAS 90 (13): 5881-5888; the entire contents of which are incorporated herein by reference). Some enediynes are capable of undergoing Bergman cyclization, and the resulting diradical, a 1,4-dehydrobenzene derivative, is capable of abstracting hydrogen atoms from the sugar backbone of DNA which results in DNA strand cleavage (see, e.g., S. Walker; R. Landovitz; W. D. Ding; G. A. Ellestad; D. Kahne (1992). “Cleavage behavior of calicheamicin gamma 1 and calicheamicin T”. Proc Natl Acad Sci U.S.A. 89 (10): 4608-12; the entire contents of which are incorporated herein by reference). Their reactivity with DNA confers an antibiotic character to many enediynes, and some enediynes are clinically investigated as anticancer antibiotics. Nonlimiting examples of enediynes are dynemicin, neocarzinostatin, calicheamicin, esperamicin (see, e.g., Adrian L. Smith and K. C. Bicolaou, “The Enediyne Antibiotics” J. Med. Chem., 1996, 39 (11), pp 2103-2117; and Donald Borders, “Enediyne antibiotics as antitumor agents,” Informa Healthcare; 1st edition (Nov. 23, 1994, ISBN-10: 0824789385; the entire contents of which are incorporated herein by reference). In a particular embodiment, the toxin may be calicheamicin.

In certain embodiments, the toxin may be a doxorubicin. “Doxorubicin” as used herein refers to members of the family of Anthracyclines derived from Streptomyces bacterium Streptomyces peucetius var. caesius, and includes doxorubicin, daunorubicin, epirubicin and idarubicin.

In certain embodiments, the toxin may be a kinesin spindle protein inhibitor. The term “kinesin spindle protein inhibitor” refers to a compound that inhibits the kinesin spindle protein, which involves in the assembly of the bipolar spindle during cell division. Kinesin spindle protein inhibitors are being investigated for the treatment of cancer. Examples of kinesin spindle protein inhibitor include ispinesib. Further, the term “kinesin spindle protein inhibitor” includes SB715992 or SB743921 from GlaxoSmithKline and pentamidine/chlorpromazine from CombinatoRx.

In certain embodiments, the toxin may a cryptophycin, or derivative, as described in US20180078656A1, US 20210163458 A1, US20210228726A1, which are incorporated by reference.

In certain embodiments, the toxin may be sandramycin. Sandramycin is a depsipeptide that has first been isolated from Nocardioides sp. (ATCC 39419) and has been shown to have cytotoxic and anti-tumor activity.

In certain embodiments, the toxin may be a thymidine synthase (or thymidylate synthase) inhibitor. Thymidylate synthase inhibitors are chemical agents which inhibit the enzyme thymidylate synthase and have potential as an anticancer chemotherapy. This inhibition prevents the methylation of C5 of deoxyuridine monophosphate (dUMP) thereby inhibiting the synthesis of deoxythymidine monophosphate (dTMP). The downstream effect is promotion of cell death because cells would not be able to properly undergo DNA synthesis if they are lacking dTMP, a necessary precursor to dTTP. Within the present invention, the thymidylate synthase inhibitor may be, without limitation, raltitrexed, pemetrexed, nolatrexed, ZD9331, GS7904L, fluorouracil, BGC-945 and OSI-7904L.

In certain embodiments, the toxin may be an amatoxin. Amatoxins (including alpha-amanitin, beta-amanitin and amanitin) are cyclic peptides composed of 8 amino acids. They can be isolated from Amanita phalloides mushrooms or prepared from the building blocks by synthesis. Amatoxins inhibit specifically the DNA-dependent RNA polymerase II of mammalian cells, and by this transcription and protein biosynthesis of the cells affected. Inhibition of transcription in a cell causes stop of growth and proliferation. Though not covalently bound, the complex between amanitin and RNA-polymerase II is very tight (KD=3 nM). Dissociation of amanitin from the enzyme is a very slow process what makes recovery of an affected cell unlikely. When in a cell the inhibition of transcription will last too long, the cell undergoes programmed cell death (apoptosis). In one preferred embodiment, term “Amatoxin” as used herein refers to an alpha-amanitin or variant thereof as described e.g. in WO2010/115630, WO2010/115629, WO2012/119787, WO2012/041504, and WO2014/135282.

In certain embodiments, the toxin may be a camptothecin. The term “camptothecin” as used herein is intended to mean a camptothecin or camptothecin derivative that functions as a topoisomerase I inhibitor. Exemplary camptothecins include, for example, topotecan, exatecan, deruxtecan, irinotecan, DX-8951f, SN38, BN 80915, lurtotecan, 9-nitrocamptothecin and aminocamptothesin. A variety of camptothecins have been described, including camptothecins used to treat human cancer patients. Several camptothecins are described, for example, in Kehrer et al., Anticancer Drugs, 12 (2): 89-105, (2001) or Li et al., ACS Med. Chem. Lett. 2019, 10, 10, 1386-1392). In certain embodiments the camptothecin is the exatecan derivative shown as compound 10 in Li et al., ACS Med. Chem. Lett. 2019, 10, 10, 1386-1392). In certain embodiments, the camptothecin derivative is a glycinated exatecan (G-Exa; FIGS. 13 and 16).

The toxin, in the sense of the present invention may also be an inhibitor of a drug efflux transporter. Antibody-payload conjugates comprising a toxin and an inhibitor of a drug efflux transporter may have the advantage that, when internalized into a cell, the inhibitor of the drug efflux transporter prevents efflux of the toxin out of the cell. Within the present invention, the drug efflux transporter may be P-glycoprotein. Some common pharmacological inhibitors of P-glycoprotein include: amiodarone, clarithromycin, ciclosporin, colchicine, diltiazem, erythromycin, felodipine, ketoconazole, lansoprazole, omeprazole and other proton-pump inhibitors, nifedipine, paroxetine, reserpine, saquinavir, sertraline, quinidine, tamoxifen, verapamil, and duloxetine. Elacridar and CP 100356 are other common P-gp inhibitors. Zosuquidar and tariquidar were also developed with this in mind. Lastly, valspodar and reversan are other examples of such agents.

The peptide linker according to the present invention comprises at least two payloads. These two or more payloads may be identical or may be different in structure.

That is, in a particular embodiment, the invention relates to the peptide linker according to the invention, wherein the two or more payloads are identical.

Coupling two or more identical payloads to a peptide linker allows increasing the concentration of the payload in the target tissue or cell of an antibody-payload conjugate. For example, if the peptide linker of an antibody-payload conjugate comprises two or more identical toxins (resulting in a DAR>4 ADC), the concentration of the toxin in the target tissue or cell will be higher compared to a conventional DAR2 ADC. With the peptide linkers of the present invention, ADCs comprising 4, 6 or 8 identical payload molecules may be obtained.

In a particular embodiment, the invention relates to the peptide linker according to the invention, wherein at least two of the two or more payloads differ from each other.

In certain embodiments, the peptide linker of the invention may allow conjugating two different payloads to an antibody. Using a second payload allows for the development of a completely new class of antibody-payload conjugates that go beyond current therapeutic approaches with respect to efficacy and potency. Also new application fields are envisioned, for example, dual-type imaging for imaging and therapy or intra-/postoperative surgery (cf. Azhdarinia A. et al., Dual-Labeling Strategies for Nuclear and Fluorescence Molecular Imaging: A Review and Analysis. Mol Imaging Biol. 2012 June; 14(3): 261-276). For example, dual-labeled antibodies encompassing a molecular imaging agent for preoperative positron emission tomography (PET) and a near-infrared fluorescent (NIRF)-dye for guided delineation of surgical margins could greatly enhance the diagnosis, staging, and resection of cancer (cf. Houghton JL. et al., Site-specifically labeled CA19.9-targeted immunoconjugates for the PET, NIRF, and multimodal PET/NIRF imaging of pancreatic cancer. Proc Natl Acad Sci USA. 2015 Dec. 29; 112(52):15850-5). PET and NIRF optical imaging offer complementary clinical applications, enabling the non-invasive whole-body imaging to localize disease and identification of tumor margins during surgery, respectively. However, the generation of such dual-labeled probes up to date has been difficult due to a lack of suitable site-specific methods; attaching two different probes by chemical means results in an almost impossible analysis and reproducibility due to the random conjugation of the probes.

In a particular embodiment, the invention relates to the peptide linker according to the invention, wherein one payload serves for imaging/detection purposes (fluorescent molecule or radioligand) and one payload serves for treatment purposes, affording a theragnostic agent (a strategy that combines therapeutics with diagnostics).

Furthermore, in a study of Levengood M. et al., (Orthogonal Cysteine Protection Enables Homogeneous Multi-Drug Antibody—Drug Conjugates. Angewandte Chemie, Volume 56, Issue 3, Jan. 16, 2017) a dual-drug labeled antibody, having attached two different auristatin toxins (having differing physiochemical properties and exerting complementary anti-cancer activities) imparted activity in cell line and xenograft models that were refractory to ADCs comprised of the individual auristatin components. This suggests that dual-labeled ADCs enable to address cancer heterogeneity and resistance more effectively than the single, conventional ADCs alone.

Thus, in certain embodiments, the peptide linker according to the invention comprises at least two different toxins. The at least two different toxins may be any of the toxins known in the art and/or disclosed herein. In particular the two or more toxins that are coupled to the peptide linker according to the invention may have different modes of action.

In certain embodiments, the peptide linker according to the invention comprises one or more auristatin and one or more camptothecin. In certain embodiments, the peptide linker according to the invention comprises one or more MMAE molecule and one or more Exatecan or Exatecan derivative (see FIG. 21).

In certain embodiments, the peptide linker according to the invention comprises two different auristatins. In certain embodiments, the peptide linker according to the invention comprises MMAE and MMAF.

In certain embodiments, the peptide linker according to the invention comprises a toxin and a hormone. In certain embodiments, the peptide linker according to the invention comprises a toxin and cortisol. In certain embodiments, the peptide linker according to the invention comprises an auristatin and cortisol. In certain embodiments, the peptide linker according to the invention comprises MMAE and cortisol (see FIG. 22). In certain embodiments, the peptide linker according to the invention comprises an auristatin, a maytansinoid and cortisol

Since one resistance mechanism towards ADCs include the active pumping-out of the cytotoxic moiety from the cancer cell, another dual-drug application may include the additional and simultaneous delivery of a drug that specifically blocks the efflux mechanism of the cytotoxic drug. Such a dual-labeled ADC could thus help to overcome cancer resistance to the ADC more effectively than conventional ADCs.

In certain embodiments, the peptide linker according to the invention may comprise at least on toxin and at least one ligand of a receptor, preferably wherein the receptor is a receptor expressed in cancer cells. In certain embodiments, the peptide linker according to the invention may comprise at least one toxin and a folate molecule.

In certain embodiments, the peptide linker according to the invention comprises three different payloads.

In a particular embodiment, the invention relates to the peptide linker according to the invention, wherein the linker is suitable to serve as substrate for a transglutaminase.

That is, the peptide linker according to the invention is designed to function as substrate for a transglutaminase. Transglutaminases are enzymes that in nature primarily catalyze the formation of an isopeptide bond between γ-carboxamide groups (—(C═O)NH₂) of glutamine residue side chains and the ε-amino groups (—NH₂) of lysine residue side chains with subsequent release of ammonia (NH₃). However, it is known in the art that the enzyme is rather promiscuous and accepts other primary amines than the ε-amino group of lysine.

It is preferred herein that the peptide linker is conjugated to a glutamine residue of an antibody by means of a transglutaminase. As such, the peptide linker has to comprise a primary amine to serve as substrate for a transglutaminase. The primary amine is preferably comprised in the side chain of a lysine, a lysine mimetic or a lysine derivative or in an amino acid residue having the structure NH₂—(Y)—COOH, as defined herein above. The transglutaminase may be any transglutaminase defined herein, preferably a microbial transglutaminase as defined herein.

In a particular embodiment, the invention relates to an antibody-payload conjugate comprising an antibody conjugated to the peptide linker according to the invention.

That is, the present invention further encompasses an antibody-linker conjugate comprising any of the peptide linkers defined herein. It is preferred herein that the amine comprising peptide linkers according to the invention are conjugated to a glutamine residue in an antibody.

As such, in a particular embodiment, the invention relates to the antibody-payload conjugate according to the invention, wherein the peptide linker is conjugated to the antibody via an isopeptide bond formed between a γ-carboxamide group of a glutamine residue comprised in the antibody and the primary amine comprised in an amino acid residue of the peptide linker.

The term “antibody” herein is used in the broadest sense and specifically covers monoclonal antibodies, polyclonal antibodies, multispecific antibodies (e.g., bispecific antibodies) formed from at least two intact antibodies, and antibody fragments so long as they exhibit the desired biological activity. The terms “antibody” and “antibodies” broadly encompass naturally occurring forms of antibodies (e.g., IgG, IgA, IgM, IgE).

The antibody is preferably a monoclonal antibody. The antibody can be of human origin, but likewise from mouse, rat, goat, donkey, hamster, or rabbit. In case the conjugate is for therapy, a murine or rabbit antibody may optionally be chimerized or humanized.

The antibody may also be bispecific (e.g., DVD-IgG, crossMab, appended IgG—HC fusion) or biparatopic. See Brinkmann and Kontermann; Bispecific antibodies; Drug Discov Today; 2015; 20(7); p. 838-47, for an overview.

The term “antibody” further encompasses antigen-binding fragments of antibodies. Preferably, the peptide linker according to the invention is conjugated to glutamine residue 295 (Q295) in the C_{H}2 domain of an IgG antibody. Thus, it is preferred herein that the antibody or antibody fragment of the invention comprises a C_{H}2 domain.

Fragments or recombinant variants of antibodies comprising the C_{H}2 domain may be, for example,
antibody formats comprising mere heavy chain domains (shark antibodies/IgNAR (V_{H}-C_{H}1-C_{H}2-C_{H}3-C_{H}4-C_{H}5)₂ or camelid antibodies/hcIgG (V_{H}—C_{H}2-C_{H}3)₂)
scFv-Fc (VH-VL-CH2-CH3)₂
Fc fusion peptides, comprising an Fc domain and one or more receptor domains.

In a particular embodiment, the invention relates to the antibody-payload conjugate according to the invention, wherein the antibody is an IgG antibody.

By “IgG” as used herein is meant a polypeptide belonging to the class of antibodies that are substantially encoded by a recognized immunoglobulin gamma gene. In humans, IgG comprises the subclasses or isotypes IgG1, IgG2, IgG3, and IgG4. In mice, IgG comprises IgG1, IgG2a, IgG2b, IgG3. Full-length IgGs consist of two identical pairs of two immunoglobulin chains, each pair having one light and one heavy chain, each light chain comprising immunoglobulin domains VL and CL, and each heavy chain comprising immunoglobulin domains VH, Cγ1 (also called C_{H}1), Cγ2 (also called C_{H}2), and Oγ3 (also called C_{H}3). In the context of human IgG1, “C_{H}1” refers to positions 118-215, C_{H}2 domain refers to positions 231-340 and C_{H}3 domain refers to positions 341-447 according to the EU index as in Kabat. IgG1 also comprises a hinge domain which refers to positions 216-230 in the case of IgG1.

In a preferred embodiment, the antibody is an IgG1 antibody. In a particularly preferred embodiment, the antibody is a human IgG1 antibody.

In a particular embodiment, the invention relates to the antibody-payload conjugate according to the invention, wherein the peptide linker is conjugated to a glutamine residue comprised in an Fc domain of the antibody.

That is, the peptide linker according to the invention is preferably conjugated to a glutamine residue comprised in an Fc domain of an antibody. The linkers of the invention may be conjugated to any Gln residue in the Fc domain of an antibody that can serve as a substrate for a transglutaminase. Typically, the term Fc domain as used herein refers to the last two constant region immunoglobulin domains of IgA, IgD and IgG (C_{H}2 and C_{H}3) and the last three constant region domains of IgE, IgY and IgM (C_{H}2, C_{H}3 and C_{H}4). That is, the linker according to the invention may be conjugated to the C_{H}2, C_{H}3 and, where applicable, C_{H}4 domains of the antibody.

For example, the peptide linker according to the invention may be conjugated to an endogenous glutamine residue (e.g., Q295 of an IgG1 antibody) or to a glutamine residue that has been introduced into the Fc domain of the antibody be genetic engineering.

That is, in a particular antibody, the invention relates to the antibody-payload conjugate according to the invention, wherein the glutamine residue to which the peptide linker is conjugated is glutamine residue Q295 (EU numbering) of the C_{H}2 domain of an IgG antibody.

It is important to understand that Q295 is an extremely conserved amino acid residue in IgG type antibodies. It is conserved in human IgG1, 2, 3, 4, as well as in rabbit and rat antibodies amongst others. Hence, being able to use Q295 is a considerable advantage for making therapeutic antibody-payload conjugates, or diagnostic conjugates where the antibody is often of non-human origin. The method according to the invention does hence provide an extremely versatile and broadly applicable tool. Even though residue Q295 is extremely conserved among IgG type antibodies, some IgG type antibodies do not possess this residue, such as mouse and rat IgG2a antibodies. Thus, it is to be understood that the antibody used in the method of the present invention is preferably an IgG type antibody comprising residue Q295 (EU numbering) of the C_{H}2 domain.

In the literature discussing the conjugation of linkers to a C_{H}2 Gln residue by means of a transglutaminase, the focus has been on small, low-molecular weight substrates. However, in the prior art literature, to accomplish such conjugation, deglycosylation of the asparagine residue at position N297, or the use of an aglycosylated antibody, has been described as necessary (WO 2015/015448; WO 2017/025179; WO 2013/092998).

Quite surprisingly, and against all expectations, however, site-specific conjugation to Q295 of glycosylated antibodies is indeed efficiently possible by using the above discussed peptide linker structure. In particular, coupling of peptide linkers comprising two or more payloads was achieved with, for most of them, a conjugation efficiency greater than 90%.

Even though Q295 is very close to N297, which is, in its native state, glycosylated, the method according to the invention, using the specified peptide linker, still allows efficient conjugation to Q295.

As shown, the method according to the invention does not require an upfront enzymatic deglycosylation of N297, nor the use of an aglycosylated antibody, nor a substitution of N297 against another amino acid, nor the introduction of a T299A mutation to prevent glycosylation.

These two points provide significant advantages under manufacturing aspects. An enzymatic deglycosylation step is undesired under GMP aspects, because it has to be made sure that the both the deglycosylation enzyme (e.g., PNGase F) as well as the cleaved glycan have to be removed from the medium.

Furthermore, no genetic engineering of the antibody for payload attachment is necessary, so that sequence insertions which may increase immunogenicity and decrease the overall stability of the antibody can be avoided.

The substitution of N297 against another amino acid may have unwanted effects, as it may affect the overall stability of the entire Fc domain (Subedi et al, The Structural Role of Antibody N-Glycosylation in Receptor Interactions. Structure 2015, 23 (9), 1573-1583), and the efficacy of the entire conjugate as a consequence that can lead to increased antibody aggregation and a decreased solubility (Zheng et al.; The impact of glycosylation on monoclonal antibody conformation and stability. Mabs-Austin 2011, 3 (6), 568-576). Further, the glycan that is present at N297 has important immunomodulatory effects, as it triggers antibody dependent cellular cytotoxicity (ADCC) and the like. These immunomodulatory effects would get lost upon deglycosylation or any of the other approaches discussed above to obtain an aglycosylated antibody. Further, any sequence modification of an established antibody can also lead to regulatory problems, which is problematic because very often an accepted and clinically validated antibody is used as a starting point for ADC conjugation.

Hence, the method according to the invention using the peptide linkers of the invention allows to easily and without disadvantages make stoichiometrically well-defined ADCs with site specific payload binding.

In view of the above, the method of the present invention is preferably used for the conjugation of an IgG antibody at residue Q295 (EU numbering) of the C_{H}2 domain of the antibody, wherein the antibody is glycosylated at residue N297 (EU numbering) of the C_{H}2 domain. However, it is expressly stated that the method of the invention also encompasses the conjugation of deglycosylated or aglycosylated antibodies at residue Q295 or any other suitable Gln residue of the antibody, wherein the Gln residue may be an endogenous Gln residue or a Gln residue that has been introduced by molecular engineering.

Thus, in a particular embodiment, the invention relates to the antibody-payload conjugate according to the invention, wherein the glutamine residue to which the peptide linker is conjugated has been introduced into the heavy or light chain of the antibody by molecular engineering.

The term “molecular engineering,” as used herein, refers to the use of molecular biology methods to manipulate nucleic acid sequences. Within the present invention, molecular engineering may be used to introduce Gln residues into the heavy or light chain of an antibody. In general, two different strategies to introduce Gln residues into the heavy or light chain of an antibody are envisioned within the present invention. First, single residues of the heavy or light chain of an antibody may be substituted with a Gln residue. Second, Gln-containing peptide tags consisting of two or more amino acid residues may be integrated into the heavy or light chain of an antibody. For that, the peptide tag may either be integrated into an internal position of the heavy or light chain, that is, between two existing amino acid residues of the heavy or light chain or by replacing them, or the peptide tag may be fused (appended) to the N- or C-terminal end of the heavy or light chain of the antibody.

For example, an amino residue of the heavy or light chain of an antibody may be substituted with a Gln residue, provided that the resulting antibody can be conjugated with the linkers of the invention by a microbial transglutaminase. In certain embodiments, the antibody is an antibody wherein amino acid residue N297 (EU numbering) of the C_{H}2 domain of an IgG antibody is substituted, in particular wherein the substitution is an N297Q substitution. Antibodies comprising an N297Q mutation may be conjugated to more than one linker per heavy chain of the antibody. For example, antibodies comprising an N297Q mutation may be conjugated to four linkers, wherein one linker is conjugated to residue Q295 of the first heavy chain of the antibody, one linker is conjugated to residue N297Q of the first heavy chain of the antibody, one linker is conjugated to residue Q295 of the second heavy chain of the antibody and one linker is conjugated to residue N297Q of the second heavy chain of the antibody. The skilled person is aware that replacement of residue N297 of an IgG antibody with a Gln residue results in an aglycosylated antibody.

That is, in a particular embodiment, the invention relates to the antibody-payload conjugate according to the invention, wherein the glutamine residue that has been introduced into the heavy or light chain of the antibody by molecular engineering is N297Q (EU numbering) of the CH2 domain of an aglycosylated IgG antibody.

In a particular embodiment, the invention relates to the antibody-payload conjugate according to the invention, wherein the glutamine residue that has been introduced into the heavy or light chain of the antibody by molecular engineering is comprised in a peptide that has been (a) integrated into the heavy or light chain of the antibody or (b) fused to the N- or C-terminal end of the heavy or light chain of the antibody.

Instead of substituting single amino acid residues of an antibody, peptide tags comprising a Gln residue that is accessible for a transglutaminase may be introduced into the heavy or light chain of the antibody. Such peptide tags may be fused to the N- or C-terminus of the heavy or light chain of the antibody. Alternatively, peptide tags may be inserted into the heavy or light chain of an antibody at a suitable position. Preferably, peptide tags comprising a transglutaminase-accessible Gln residue are fused to the C-terminus of the heavy chain of the antibody. Even more preferably, the peptide tags comprising a transglutaminase-accessible Gln residue are fused to the C-terminus of the heavy chain of an IgG antibody. Several peptide tags that may be fused to the C-terminus of the heavy chain of an antibody and serve as substrate for a microbial transglutaminase are described in WO 2012/059882 and WO 2016/144608.

Thus, in a particular embodiment, the invention relates to the antibody-payload conjugate according to the invention, wherein the peptide comprising the Gln residue has been fused to the C-terminal end of the heavy chain of the antibody.

Exemplary peptide tags that may be introduced into the heavy or light chain of an antibody, in particular fused to the C-terminus of the heavy chain of the antibody, are LLQGG (SEQ ID NO:70), LLQG (SEQ ID NO:37), LSLSQG (SEQ ID NO:38), GGGLLQGG (SEQ ID NO:39), GLLQG (SEQ ID NO:40), LLQ (SEQ ID NO:41), GSPLAQSHGG (SEQ ID NO:42), GLLQGGG (SEQ ID NO:43), GLLQGG (SEQ ID NO:44), GLLQ (SEQ ID NO:45), LLQLLQGA (SEQ ID NO:46), LLQGA (SEQ ID NO:47), LLQYQGA (SEQ ID NO:48), LLQGSG (SEQ ID NO:49), LLQYQG (SEQ ID NO:50), LLQLLQG (SEQ ID NO:51), SLLQG (SEQ ID NO:52), LLQLQ (SEQ ID NO:53), LLQLLQ (SEQ ID NO:54), LLQGR (SEQ ID NO:55), EEQYASTY (SEQ ID NO:56), EEQYQSTY (SEQ ID NO:57), EEQYNSTY (SEQ ID NO:58), EEQYQS (SEQ ID NO:59), EEQYQST (SEQ ID NO:60), EQYQSTY (SEQ ID NO:61), QYQS (SEQ ID NO:62), QYQSTY (SEQ ID NO:63), YRYRQ (SEQ ID NO:64), DYALQ (SEQ ID NO:65), FGLQRPY (SEQ ID NO:66), EQKLISEEDL (SEQ ID NO:67), LQR (SEQ ID NO:68) and YQR (SEQ ID N0:69).

The skilled person is aware of methods to substitute amino acid residues of antibodies or to introduce peptide tags into antibodies, for example by methods of molecular cloning as described in Sambrook, Joseph. (2001). Molecular cloning: a laboratory manual. Cold Spring Harbor, N.Y.: Cold Spring Harbor Laboratory Press.

In general, the skilled person is aware of methods to determine at which position of an antibody a peptide linker is conjugated. For example, the conjugation site may be determined by proteolytic digestion of the antibody-payload conjugate and LC-MS analysis of the resulting fragments. For example, samples may be deglycosylated with GlyciNATOR (Genovis) according to the instruction manual and subsequently digested with trypsin gold (mass spectrometry grade, Promega), respectively. Therefore, 1 μg of protein may be incubated with 50 ng trypsin at 37° C. overnight. LC-MS analysis may be performed using a nanoAcquity HPLC system coupled to a Synapt-G2 mass spectrometer (Waters). For that, 100 ng peptide solution may be loaded onto an Acquity UPLC Symmetry C18 trap column (Waters, part no. 186006527) and trapped with 5 μL/min flow rate at 1% buffer A (Water, 0.1% formic acid) and 99% buffer B (acetonitrile, 0.1% formic acid) for 3 min. Peptides may then be eluted with a linear gradient from 3% to 65% Buffer B within 25 min. Data may be acquired in resolution mode with positive polarity and in a mass range from 50 to 2000 m/z. Other instrument settings may be as follows: capillary voltage 3.2 kV, sampling cone 40 V, extraction cone 4.0 V, source temperature 130° C., cone gas 35 L/h, nano flow gas 0.1 bar, and purge gas 150 L/h. The mass spectrometer may be calibrated with [Glu1]-Fibrinopeptide.

Further, the skilled person is aware of methods to determine the drug-to-antibody (DAR) ratio or payload-to-antibody ratio of an antibody-payload construct. For example, the DAR may be determined by hydrophobic interaction chromatography (HIC) or LC-MS.

For hydrophobic interaction chromatography (HIC), samples may be adjusted to 0.5 M ammonium sulfate and assessed via a MAB PAK HIC Butyl column (5 μm, 4.6×100 mm, Thermo Scientific) using a full gradient from A (1.5 M ammonium sulfate, 25 mM Tris HCl, pH 7.5) to B (20% isopropanol, 25 mM Tris HCl, pH 7.5) over 20 min at 1 mL/min and 30° C. Typically, 40 μg sample may be used and signals may be recorded at 280 nm. Relative HIC retention times (HIC-RRT) may be calculated by dividing the absolute retention time of the ADC DAR 2 species by the retention time of the respective unconjugated mAb.

For LC-MS DAR determination, ADCs may be diluted with NH₄HCO₃ to a final concentration of 0.025 mg/mL. Subsequently, 40 μL of this solution may be reduced with 1 μL TCEP (500 mM) for 5 min at room temperature and then alkylated by adding 10 μL chloroacetamide (200 mM), followed by overnight incubation at 37° C. in the dark. For reversed phase chromatography, a Dionex U3000 system in combination with the software Chromeleon may be used. The system may be equipped with a RP-1000 column (1000 Å, 5 μm, 1.0×100 mm, Sepax) heated to 70° C., and an UV-detector set to a wavelength of 214 nm. Solvent A may consist of water with 0.1% formic acid and solvent B may comprise 85% acetonitrile with 0.1% formic acid. The reduced and alkylated sample may be loaded onto the column and separated by a gradient from 30-55% solvent B over the course of 14 min. The liquid chromatography system may be coupled to a Synapt-G2 mass spectrometer for identification of the DAR species. The capillary voltage of the mass spectrometer may be set to 3 kV, the sampling cone to 30 V and the extraction cone may add up to a value of 5 V. The source temperature may be set to 150° C., the desolvation temperature to 500° C., the cone gas to 20 l/h, the desolvation gas to 600 l/h, and the acquisition may be made in positive mode in a mass range from 600-5000 Da with 1 s scan time. The instrument may be calibrated with sodium iodide. Deconvolution of the spectra may be performed with the MaxEnt1 algorithm of MassLynx until convergence. After assignment of the DAR species to the chromatographic peaks, the DAR may be calculated based on the integrated peak areas of the reversed phase chromatogram.

In a particular embodiment, the invention relates to the antibody-payload conjugate according to the invention, wherein the IgG antibody is a glycosylated IgG antibody.

That is, it is preferred herein that the peptide linker according to the invention is conjugated to a glycosylated IgG antibody. It is particularly preferred that the peptide linker according to the invention is conjugated to a native glycosylated IgG antibody. Native IgG antibodies comprise a single conjugation site at glutamine residue 295 (Q295). Thus, it is particularly preferred herein that the peptide linker according to the invention is conjugated to residue Q295 of a native glycosylated antibody. The only glycosylation site of native IgG antibodies is asparagine residue 297 (N297).

Thus, in a particular embodiment, the invention relates to the antibody-payload conjugate according to the invention, wherein the IgG antibody is glycosylated at residue N297 (EU numbering) of the CH2 domain.

In a particularly preferred embodiment, the peptide linker according to the invention is conjugated to position Q295 of an IgG antibody that is glycosylated at position N297. More preferably, the antibody is an IgG1 antibody.

In a particular embodiment, the invention relates to the antibody-payload conjugate according to the invention, wherein the antibody is selected from the group consisting of: Brentuximab (anti-CD30), Trastuzumab (anti-Her2/neu), Gemtuzumab (anti-CD33), Inotuzumab (anti-CD22), Avelumab (anti-PD-L1), Cetuximab (anti-EGFR), Rituximab (anti-CD20), Daratumumab (anti-CD38), Pertuzumab (anti-HER2), Vedolizumab (anti-Integrin α4β7), Ocrelizumab (anti-CD20), Tocilizumab (anti-IL-6-R), Ustekinumab (anti-IL-12/23), Golimumab (anti-TNFα), Obinutuzumab (anti-CD20), Sacituzumab (anti-Trop-2), Belantamab (anti-BCMA), Polatuzumab (anti-CD79b), Enfortumab (anti-Nectin-4), Endrecolomab (anti-EpCAM), Gemtuzumab (anti-CD33), Loncastuximab (anti-CD19), Mecbotamab (anti-AXL), Adecatumumab (anti-EpCAM), D93 (anti-dn-collagen), Gatipotuzumab (anti-TA-MUC1), Labetuzumab (anti-carcinoembryonic cell adhesion molecule 5), Tusamitamab (anti-CEACAM5), Upifitamab (anti-NaPi2b), Lifastuzumab (anti-NaPi2b), Mirvetuximab (anti-FRα)), Sofituzumab (anti-MUC16), Anetumab (anti-mesothelin), Tisotumab (anti-TF), Cofituzumab (anti-Trop-2), Praluzatamab (anti-CD166), Ladriatuzumab (anti-LIV-1), Belantamab (anti-BCMA), Patritumab (anti-ERBB3), Cetuximab (anti-EGFR), Nimotuzumab (anti-EGFR), Matuzumab (anti-EGFR), Portuzumab (anti-HER2), Citatuzumab (anti-TACSTD1), Tucotuzumab (anti-EpCAM) and Endrecolomab (anti-EpCAM).

In a particular embodiment, the invention relates to the antibody-payload conjugate according to the invention, wherein the antibody is selected from the group consisting of: Brentuximab (anti-CD30), Gemtuzumab (anti-CD30), Trastuzumab (anti-Her2/neu), Inotuzumab (anti-CD22), Polatuzumab (anti-CD79b), Enfortumab (anti-Nectin-4), Sacituzumab (anti-Trop-2) and Belantamab (anti-BCMA).

In a particular embodiment, the invention relates to the antibody-payload conjugate according to the invention, wherein the antibody is Polatuzumab (anti-CD79b) or Trastuzumab (anti-Her2/neu) or Enfortumab (anti-Nectin-4).

In certain embodiments, the invention relates to the antibody-payload conjugate according to the invention, wherein the antibody specifically binds to an antigen selected from the group consisting of: CD30, Her2/neuCD33, CD22, PD-L1, EGFR, CD20, CD38, HER2, Integrin α4β7, CD20, IL-6-R, IL-12, IL-23, TNFα, CD20, Trop-2, BCMA, CD79b, Nectin-4, EpCAM, CD33, CD19, AXL, dn-collagen, TA-MUC1, carcinoembryonic cell adhesion molecule 5, CEACAM5, NaPi2b, FRα, MUC16, mesothelin, TF, CD166, LIV-1, ERBB3, EGFR, and TACSTD1, preferably, CD30, Her2/neu, CD22, CD79b, Nectin-4, Trop-2 and BCMA, more preferably, CD79b, Her2/neu, and Nectin-4.

In a particular embodiment, the invention relates to a method for the preparation of an antibody-payload conjugate comprising a step of conjugating a peptide linker according to the invention to an antibody.

That is, any of the peptide linkers comprising two or more payloads as disclosed herein may be conjugated to an antibody. In particular, any of the amine-comprising peptide linkers disclosed herein may be conjugated to a glutamine residue of an antibody via a transglutaminase. As disclosed elsewhere herein, the glutamine residue to which the peptide linker is conjugated may be an endogenous glutamine residue (e.g., Q295 of an IgG antibody) or may be a glutamine residue that has been introduced into the antibody by molecular engineering.

In a particular embodiment, the invention relates to a method for the conjugation of a peptide linker comprising two or more payloads to an antibody using a transglutaminase (TG), the method comprising (a) mixing the antibody, the peptide linker and the TG within a fluid, thereby conjugating the linker-payload to the antibody in one step under the catalyzing effect of the TG, and (b) extracting the conjugate obtained in step (a) from the fluid.

Accordingly, the present invention further encompasses methods for conjugating peptide linkers comprising two or more payloads to an antibody by means of a transglutaminase in a one-step reaction. For that, an antibody may be mixed with the peptide linker according to the invention and a transglutaminase within a fluid. A “fluid”, within the meaning of the present invention is a liquid. Preferably, the liquid is an aqueous solution, even more preferably a buffered aqueous solution.

The peptide linker according to the invention may be mixed with the antibody and the transglutaminase by mixing a solution comprising said peptide linker with a solution comprising an antibody and a solution comprising the transglutaminase. Alternatively, solutions individually comprising the peptide linker, the antibody and the transglutaminase may be added to an aqueous solution. In particular, each component may be added to the aqueous solution at a defined concentration. The peptide linker according to the invention is conjugated to the antibody under the catalyzing effect of the transglutaminase. That is, the individual components may be mixed under conditions that are suitable for an efficient conjugation of the peptide linker to the antibody. Such conditions are defined elsewhere herein.

In a second method step, the obtained antibody-payload conjugates have to be removed from the liquid. The skilled person is aware of methods to isolate antibody-payload conjugates from an aqueous solution. Further, the skilled person is aware of methods to separate antibody-payload conjugates from unconjugated antibodies or peptide linkers or from incompletely conjugated antibodies. For example, antibody payload conjugates according to the invention may be isolated from the mixture by HPLC.

It is to be understood that “extracting the conjugate from the fluid” is synonymous with “isolating the conjugate from the mixture”. That is, a conjugate may also be extracted by removing the transglutaminase and unconjugated antibody and peptide linker from the fluid.

The peptide linker that is used in the method according to the invention may be any one of the peptide linkers disclosed herein, in particular any peptide linker falling within the definition provided herein above or any peptide linker shown in the experimental examples.

Thus, in a particular embodiment, the invention relates to the method according to the invention, wherein the peptide linker is the peptide linker of the invention.

Further, the antibody may be an antibody as defined in more detail elsewhere herein, i.e., for the antibody-payload conjugate according to the invention.

In particular, the peptide linker may comprise an amino acid sequence as set forth in SEQ ID NOs:1-29 or 82-93. Furthermore, the linker may be any one of the linkers shown in FIGS. 1-40 or 42-43.

That is, in a particular embodiment, the invention relates to the method according to the invention, wherein the peptide linker is conjugated to a glutamine residue comprised in the antibody via a primary amine comprised in an amino acid residue of the peptide linker.

In a particular embodiment, the invention relates to the method according to the invention, wherein the antibody is an antibody fragment.

In a particular embodiment, the invention relates to the method according to the invention, wherein the antibody is an IgA, IgD, IgE, IgG or IgM antibody.

In a particular embodiment, the invention relates to the method according to the invention, wherein the peptide linker is conjugated to a glutamine residue comprised in an Fc domain of the antibody.

In a particular embodiment, the invention relates to the method according to the invention, wherein the glutamine residue to which the peptide linker is conjugated is glutamine residue Q295 (EU numbering) of the C_{H}2 domain of an IgG antibody.

In a particular embodiment, the invention relates to the method according to the invention, wherein the glutamine residue to which the peptide linker is conjugated has been introduced into the heavy or light chain of the antibody by molecular engineering.

In a particular embodiment, the invention relates to the method according to the invention, wherein the glutamine residue that has been introduced into the heavy or light chain of the antibody by molecular engineering is N297Q (EU numbering) of the C_{H}2 domain of an aglycosylated IgG antibody.

In a particular embodiment, the invention relates to the method according to the invention, wherein the glutamine residue that has been introduced into the heavy or light chain of the antibody by molecular engineering is comprised in a peptide that has been (a) integrated into the heavy or light chain of the antibody or (b) fused to the N- or C-terminal end of the heavy or light chain of the antibody.

In a particular embodiment, the invention relates to the method according to the invention, wherein the peptide comprising the Gln residue has been fused to the C-terminal end of the heavy chain of the antibody.

In a particular embodiment, the invention relates to the method according to the invention, wherein the antibody is a glycosylated IgG antibody.

In a particular embodiment, the invention relates to the method according to the invention, wherein the IgG antibody is glycosylated at residue N297 (EU numbering) of the C_{H}2 domain.

In a particular embodiment, the invention relates to the method according to the invention, wherein the antibody is selected from the group consisting of: Brentuximab (anti-CD30), Trastuzumab (anti-Her2/neu), Gemtuzumab (anti-CD33), Inotuzumab (anti-CD22), Avelumab (anti-PD-L1), Cetuximab (anti-EGFR), Rituximab (anti-CD20), Daratumumab (anti-CD38), Pertuzumab (anti-HER2), Vedolizumab (anti-Integrin α4β7), Ocrelizumab (anti-CD20), Tocilizumab (anti-IL-6-R), Ustekinumab (anti-IL-12/23), Golimumab (anti-TNFα), Obinutuzumab (anti-CD20), Sacituzumab (anti-Trop-2), Belantamab (anti-BCMA), Polatuzumab (anti-CD79b), Enfortumab (anti-Nectin-4), Endrecolomab (anti-EpCAM), Gemtuzumab (anti-CD33), Loncastuximab (anti-CD19), Mecbotamab (anti-AXL), Adecatumumab (anti-EpCAM), D93 (anti-dn-collagen), Gatipotuzumab (anti-TA-MUC1), Labetuzumab (anti-carcinoembryonic cell adhesion molecule 5), Tusamitamab (anti-CEACAM5), Upifitamab (anti-NaPi2b), Lifastuzumab (anti-NaPi2b), Mirvetuximab (anti-FRα)), Sofituzumab (anti-MUC16), Anetumab (anti-mesothelin), Tisotumab (anti-TF), Cofituzumab (anti-Trop-2), Praluzatamab (anti-CD166), Ladriatuzumab (anti-LIV-1), Belantamab (anti-BCMA), Patritumab (anti-ERBB3), Cetuximab (anti-EGFR), Nimotuzumab (anti-EGFR), Matuzumab (anti-EGFR), Portuzumab (anti-HER2), Citatuzumab (anti-TACSTD1), Tucotuzumab (anti-EpCAM) and Endrecolomab (anti-EpCAM).

In a particular embodiment, the invention relates to the method according to the invention, wherein the antibody is selected from the group consisting of: Brentuximab (anti-CD30), Gemtuzumab (anti-CD30), Trastuzumab (anti-Her2/neu), Inotuzumab (anti-CD22), Polatuzumab (anti-CD79b), Enfortumab (anti-Nectin-4), Sacituzumab (anti-Trop-2) and Belantamab (anti-BCMA).

In a particular embodiment, the invention relates to the method according to the invention, wherein the antibody is Polatuzumab (anti-CD79b) or Trastuzumab (anti-Her2/neu) or Enfortumab (anti-Nectin-4).

In certain embodiments, the invention relates to the method according to the invention, wherein the antibody specifically binds to an antigen selected from the group consisting of: CD30, Her2/neuCD33, CD22, PD-L1, EGFR, CD20, CD38, HER2, Integrin α4β7, CD20, IL-6-R, IL-12, IL-23, TNFα, CD20, Trop-2, BCMA, CD79b, Nectin-4, EpCAM, CD33, CD19, AXL, dn-collagen, TA-MUC1, carcinoembryonic cell adhesion molecule 5, CEACAM5, NaPi2b, FRα, MUC16, mesothelin, TF, CD166, LIV-1, ERBB3, EGFR, and TACSTD1, preferably, CD30, Her2/neu, CD22, CD79b, Nectin-4, Trop-2 and BCMA, more preferably, CD79b, Her2/neu, and Nectin-4.

In a particular embodiment, the invention relates to the method according to the invention, wherein the peptide linker is conjugated to a γ-carboxamide group of a Gln residue comprised in the antibody.

In a particular embodiment, the invention relates to the method according to the invention, wherein the peptide linker is suitable for conjugation to a glycosylated antibody with a conjugation efficiency of at least 20%, 30%, 40%, 50%, 60%, 70%, 75%, 80%, 85%, 90% or 95%.

That is, in certain embodiments, the peptide linker according to the invention may be conjugated to a glycosylated antibody with an efficiency of at least 20%, 30%, 40%, 50%, 60%, 70%, 75%, 80%, 85%, 90% or 95%. In a preferred embodiment, the peptide linker according to the invention may be conjugated to a glycosylated antibody with an efficiency of at least 70%. In another preferred embodiment, the peptide linker according to the invention may be conjugated to a glycosylated antibody with an efficiency of at least 75%. In another preferred embodiment, the peptide linker according to the invention may be conjugated to a glycosylated antibody with an efficiency of at least 80%. In another preferred embodiment, the peptide linker according to the invention may be conjugated to a glycosylated antibody with an efficiency of at least 85%. In another preferred embodiment, the peptide linker according to the invention may be conjugated to a glycosylated antibody with an efficiency of at least 90%. In another preferred embodiment, the peptide linker according to the invention may be conjugated to a glycosylated antibody with an efficiency of at least 95%. Preferably, the glycosylated antibody is a glycosylated IgG antibody, more preferably an IgG antibody that is glycosylated at residue N297 (EU numbering).

The skilled person is aware of methods to determine the conjugation efficiency of an antibody with a specific peptide linker. For example, the conjugation efficiency may be determined as described herein. That is, an antibody, in particular an IgG1 antibody, may be incubated at a concentration of 1-5 mg/mL with 5-20 eq molar equivalents of a linker and 3-6 U of a microbial transglutaminase per mg of antibody in a suitable buffer for 20-48 hours at 37° C. or as described in Example 1. After the incubation period, the conjugation efficiency may be determined by LC-MS analysis under reducing conditions. The microbial transglutaminase may be an MTG from Streptomyces mobaraensis that is, for example, available from Zedira (Germany). A suitable buffer may be a Tris, MOPS, HEPES, PBS or BisTris buffer. However, it is to be understood that the choice of the buffer system may vary and depend to a large extent on the chemical properties of the linker. However, the skilled person is capable of identifying the optimal buffer conditions based on the disclosure of the present invention. Alternatively, the conjugation efficiency may be determined as described in Spycher et al. (Dual, Site-Specific Modification of Antibodies by Using Solid-Phase Immobilized Microbial Transglutaminase, ChemBioChem 2019 18(19):1923-1927) and analyzed as in Benjamin et al. (Thiolation of Q295: Site-Specific Conjugation of Hydrophobic Payloads without the Need for Genetic Engineering, Mol. Pharmaceutics 2019, 16: 2795-2807).

In certain embodiments, antibodies may be conjugated by incubating 5 mg/ml of native, glycosylated monoclonal antibody for 24 hours at 37° C. in 50 mM Tris pH 7.6 with a microbial transglutaminase (MTG, Zedira) at a concentration of 5-10 U/mg antibody and 5 molar equivalents of the indicated linker-payload in a rotating thermomixer. However, it is to be understood that the conditions, in particular the buffer conditions and the peptide linker concentration may be adjusted depending on the properties of the payload(s). However, the skilled person is able to identify the optimal reaction conditions based on the teaching provided herein.

In a particular embodiment, the invention relates to the method according to the invention, wherein the transglutaminase is a microbial transglutaminase (MTG).

The transglutaminase for use in the method of the present invention may be any transglutaminase that is suitable for conjugating the peptide linker of the invention to an antibody. The transglutaminase may be of any origin, e.g., the transglutaminase may be of bacterial, archaeal or eukaryotic origin.

In certain embodiments, the transglutaminase may be a mammalian transglutaminase, including human transglutaminases. In certain embodiments, the transglutaminase may be a microbial transglutaminase, including bacterial and fungal transglutaminases.

In a particular embodiment, the invention relates to the method according to the invention, wherein the microbial transglutaminase is derived from a Streptomyces species, in particular Streptomyces mobaraensis.

That is, the microbial transglutaminase used in the method of the invention may be derived from a Streptomyces species, in particular from Streptomyces mobaraensis, preferentially with a sequence identity of 80% to the native enzyme. Accordingly, the MTG may be a native enzyme or may be an engineered variant of a native enzyme.

One such microbial transglutaminase is commercially available from Zedira (Germany). It is recombinantly produced in E. coli. Streptomyces mobaraensis transglutaminase has an amino acid sequence as disclosed in SEQ ID NO:78. S. mobaraensis MTG variants with other amino acid sequences have been reported and are also encompassed by this invention (SEQ ID NO:79 and 80).

One such microbial transglutaminase could also be the MTG-TX variant from S. mobaraensis described in Jn et al. 2016, Journal of Molecular Catalysis B: Enzymatic, which exhibits high-salt-resistance and a broad range of pH and temperature stability.

In another embodiment, a microbial transglutaminase from Streptomyces ladakanum (formerly known as Streptoverticillium ladakanum) may be used. Streptomyces ladakanum transglutaminase (U.S. Pat. No. 6,660,510 B2) has an amino acid sequence as disclosed in SEQ ID NO:81.

Both of the above transglutaminases may be sequence modified. In several embodiments, transglutaminases may be used which have 80%, 85%, 90% or 95% or more sequence identity with any one of SEQ ID NO:78-81.

Another suitable microbial transglutaminase is commercially from Ajinomoto, called ACTIVA TG. In comparison to the transglutaminase from Zedira, ACTIVA TG lacks 4 N-terminal amino acids, but has similar activity.

Further microbial transglutaminases which may be used in the context of the present invention are disclosed in Kieliszek and Misiewicz (Folia Microbiol (Praha). 2014; 59(3): 241-250), WO 2015/191883 A1, WO 2008/102007 A1 and US 2010/0143970, the contents of which is fully incorporated herein by reference.

In certain embodiments, a mutant variant of a microbial transglutaminase may be used for the conjugation of a linker to an antibody. That is, the microbial transglutaminase that is used in the method of the present invention may be a variant of S. mobaraensis transglutaminase as set forth in SEQ ID NOs: 78 or 79. In certain embodiments, the recombinant S. mobaraensis transglutaminase as set forth in SEQ ID NO:78 may comprise the mutation G254D. In certain embodiments, the recombinant S. mobaraensis transglutaminase as set forth in SEQ ID NO:78 may comprise the mutations G254D and E304D. In certain embodiments, the recombinant S. mobaraensis transglutaminase as set forth in SEQ ID NO:78 may comprise the mutations D8E and G254D. In certain embodiments, the recombinant S. mobaraensis transglutaminase as set forth in SEQ ID NO:78 may comprise the mutations E124A and G254D. In certain embodiments, the recombinant S. mobaraensis transglutaminase as set forth in SEQ ID NO:78 may comprise the mutations A216D and G254D. In certain embodiments, the recombinant S. mobaraensis transglutaminase as set forth in SEQ ID NO:78 may comprise the mutations G254D and K331T.

In a particular embodiment, the invention relates to the method according to the invention, wherein the transglutaminase is added to the conjugation reaction at a concentration of less than 200 U/mg antibody.

Microbial transglutaminase may be added to the conjugation reaction at any concentration that allows efficient conjugation of an antibody with a linker. In certain embodiments, the concentration of microbial transglutaminase in a conjugation reaction may depend on the amount of antibody used in the same reaction. For example, a microbial transglutaminase may be added to the conjugation reaction at a concentration of less than 200 U/mg antibody, 150 U/mg antibody 100 U/mg antibody, 90 U/mg antibody, 80 U/mg antibody, 70 U/mg antibody, 60 U/mg antibody, 50 U/mg antibody, 40 U/mg antibody, 30 U/mg antibody, 20 U/mg antibody 10 U/mg antibody or 6 U/mg antibody.

In certain embodiments a microbial transglutaminase may be added to the conjugation reaction at a concentration of 1 U/mg antibody. In certain embodiments a microbial transglutaminase may be added to the conjugation reaction at a concentration of 3 U/mg antibody. In certain embodiments a microbial transglutaminase may be added to the conjugation reaction at a concentration of 5 U/mg antibody. In certain embodiments a microbial transglutaminase may be added to the conjugation reaction at a concentration of 6 U/mg antibody. In certain embodiments a microbial transglutaminase may be added to the conjugation reaction at a concentration of 7.5 U/mg antibody. In certain embodiments a microbial transglutaminase may be added to the conjugation reaction at a concentration of 10 U/mg antibody.

In certain embodiments a microbial transglutaminase may be added to the conjugation reaction at a concentration of 1-100 U/mg antibody. In certain embodiments a microbial transglutaminase may be added to the conjugation reaction at a concentration of 3-50 U/mg antibody. In certain embodiments a microbial transglutaminase may be added to the conjugation reaction at a concentration of 5-25 U/mg antibody.

In certain embodiments a microbial transglutaminase may be added to the conjugation reaction at a concentration of 1-20 U/mg antibody, preferably at a concentration of 3-15 U/mg antibody, more preferably at a concentration of 5-10 U/mg antibody.

Preferably, the transglutaminase for use in the method of the invention is a microbial transglutaminase. However, it is to be noted that an equivalent reaction may be carried out by an enzyme comprising transglutaminase activity that is of a non-microbial origin. Accordingly, also the antibody-payload conjugates according to the invention may be generated with an enzyme comprising transglutaminase activity that is of a non-microbial origin.

In a particular embodiment, the invention relates to the method according to the invention, wherein the antibody is added to the conjugation reaction at a concentration of 0.1-50 mg/mL.

The antibody may be added to the conjugation reaction at any concentration that is suitable for obtaining efficient conjugation of the antibody. However, it is preferred that the antibody is added to the conjugation reaction at a concertation ranging from 0.1-50 mg/ml. That is, in a particular embodiment, the invention relates to the method according to the invention, wherein the antibody is added to the conjugation reaction at a concentration of 0.1-50 mg/mL, preferably 0.25-25 mg/mL, more preferably 0.5-12.5 mg/mL, even more preferably 1-10 mg/mL, even more preferably 2-7.5 mg/mL, most preferably about 5 mg/mL.

Alternatively, the antibody may be added to the conjugation reaction at a concertation ranging from 1-20 mg/ml, preferably from 2.5-20 mg/mL, more preferably from 5-20 mg/mL, most preferably from 5-17 mg/mL.

In a particular embodiment, the invention relates to the method according to the invention, wherein the antibody is contacted with 2-100 molar equivalents of peptide linker.

To obtain efficient conjugation, it is preferred that the linker is added to the antibody in molar excess. That is, in certain embodiments, the antibody is mixed with at least 2, 5, 10, 20, 30, 40, 50, 60, 70, 80, 90 or 100 molar equivalents of a linker.

That is, in a particular embodiment, the invention relates to the method according to the invention, wherein the antibody is contacted with 2-100 molar equivalents of linker, preferably 2-80 molar equivalents of linker, more preferably 2-70 molar equivalents of linker, even more preferably 2-60 molar equivalents of linker, even more preferably 2-50 molar equivalents of linker, even more preferably 2-40 molar equivalents of linker, even more preferably 2-30 molar equivalents of linker, even more preferably 2 to 25 molar equivalents of linker, even more preferably 2-20 molar equivalents of linker, even more preferably 2-15 molar equivalents of linker, most preferably 2-10 molar equivalents of linker.

Alternatively, the antibody may be contacted with 2.5-100 molar equivalents of linker, preferably 2.5-80 molar equivalents of linker, more preferably 2.5-70 molar equivalents of linker, even more preferably 2.5-60 molar equivalents of linker, even more preferably 2.5-50 molar equivalents of linker, even more preferably 2.5-40 molar equivalents of linker, even more preferably 2.5-30 molar equivalents of linker, even more preferably 2.5-20 molar equivalents of linker, even more preferably 2.5-15 molar equivalents of linker, even more preferably 2.5-10 molar equivalents of linker, most preferably 2.5-8 molar equivalents of linker.

Alternatively, the antibody may be contacted with 5-100 molar equivalents of linker, preferably 5-80 molar equivalents of linker, more preferably 5-70 molar equivalents of linker, even more preferably 5-60 molar equivalents of linker, even more preferably 5-50 molar equivalents of linker, even more preferably 5-40 molar equivalents of linker, even more preferably 5-30 molar equivalents of linker, even more preferably 5-20 molar equivalents of linker, even more preferably 5-15 molar equivalents of linker, most preferably 5-10 molar equivalents of linker.

In a particular embodiment, the invention relates to the method according to the invention, wherein the conjugation reaction is carried out in a buffered solution.

The method according to the invention is preferably carried out at a pH ranging from 5 to 10. Thus, in a preferred embodiment, the invention relates to a method according to the invention, wherein the conjugation of the linker to the antibody is achieved at a pH ranging from 5 to 10, preferably at a pH ranging from 6 to 9, more preferably at a pH ranging from 6 to 8.5, even more preferably at a pH ranging from 6.5 to 8, most preferably at a pH ranging from 6.6 to 7.6.

In certain embodiments, the invention relates to a method according to the invention, wherein the conjugation of the linker to the antibody is achieved at pH 6.6.

In certain embodiments, the invention relates to a method according to the invention, wherein the conjugation of the linker to the antibody is achieved at pH 7.6.

The method of the invention may be carried out in any buffer that is suitable for the conjugation of the payload to the linker. Buffers that are suitable for the method of the invention include, without limitation, Tris, MOPS, HEPES, PBS or BisTris buffer. The concentration of the buffer depends, amongst others, on the concentration of the antibody and/or the linker and may range from 10-1000 mM, 10-500 mM, 10-400 mM, 10 to 250 mM, 10 to 150 mM or 10 to 100 mM. Further, the buffer may comprise any salt concentration that is suitable for carrying out the method of the invention. For example, the buffer used in the method of the invention may have a salt concentration 250 mM, ≤200 mM, ≤150 mM, ≤140 mM, ≤130 mM, ≤120 mM, ≤110 mM, ≤100 mM, ≤90 mM, ≤80 mM, ≤70 mM, ≤60 mM, ≤50 mM, ≤40 mM, ≤30 mM, ≤20 mM or 10 mM or no salts.

That is, in a particular embodiment, the invention relates to the method according to the invention, wherein the buffered solution comprises
a) a pH ranging from 5 to 10; and/or
b) a buffer concentration ranging from 10 to 1000 mM; and/or
c) a salt concentration below 250 mM.

In a preferred embodiment, the invention relates to the method according to the invention, wherein the buffered solution comprises
a) a pH ranging from 6 to 9; and/or
b) a buffer concentration ranging from 10 to 1000 mM; and/or
c) a salt concentration below 250 mM.

In a more preferred embodiment, the invention relates to the method according to the invention, wherein the buffered solution comprises
a) a pH ranging from 6 to 8; and/or
b) a buffer concentration ranging from 10 to 500 mM; and/or
c) a salt concentration below 150 mM.

In an even more preferred embodiment, the invention relates to the method according to the invention, wherein the buffered solution comprises
a) a pH ranging from 6 to 8; and/or
b) a buffer concentration ranging from 10 to 200 mM; and/or
c) a salt concentration below 50 mM.

In a preferred embodiment, the method of the invention is carried out in 50 mM Tris (pH 7.6), preferably without salts.

In another preferred embodiment, the method of the invention is carried out in 50 mM BisTris (pH 6.6), preferably without salts.

In another preferred embodiment, the method of the invention is carried out in 50 mM BisTris (pH 7.5), preferably without salts.

It has to be noted that the optimal reaction conditions (e.g. pH, buffer, salt concentration) may vary between payloads and to some degree depend on the physicochemical properties of the linkers and/or payloads. However, no undue experimentation is required by the skilled person to identify reaction conditions that are suitable for carrying out the method of the invention.

It is to be understood that the application encompasses any combination of the above-disclosed linker, antibody, MTG and/or buffer concentrations.

In certain embodiments, the invention relates to the methods according to the invention, wherein the antibody is contacted with 2-80 molar equivalents of the linker; and/or wherein the microbial transglutaminase is added to the conjugation reaction at a concentration ranging from 1-20 U/mg antibody and, optionally, wherein the antibody is added to the conjugation reaction at a concentration ranging from 0.1-20 mg/mL.

In a preferred embodiment, the invention relates to the methods according to the invention, wherein the antibody is contacted with 2-50 molar equivalents of the linker; and/or wherein the microbial transglutaminase is added to the conjugation reaction at a concentration ranging from 1-15 U/mg antibody and, optionally, wherein the antibody is added to the conjugation reaction at a concentration ranging from 1-20 mg/mL.

In a more preferred embodiment, the invention relates to the methods according to the invention, wherein the antibody is contacted with 2-30 molar equivalents of the linker; and/or wherein the microbial transglutaminase is added to the conjugation reaction at a concentration ranging from 2-15 U/mg antibody and, optionally, wherein the antibody is added to the conjugation reaction at a concentration ranging from 2.5-20 mg/mL.

In an even more preferred embodiment, the invention relates to the methods according to the invention, wherein the antibody is contacted with 2-20 molar equivalents of the linker; and/or wherein the microbial transglutaminase is added to the conjugation reaction at a concentration ranging from 5-15 U/mg antibody and, optionally, wherein the antibody is added to the conjugation reaction at a concentration ranging from 2.5-20 mg/mL.

In an even more preferred embodiment, the invention relates to the methods according to the invention, wherein the antibody is contacted with 2-15 molar equivalents of the linker; and/or wherein the microbial transglutaminase is added to the conjugation reaction at a concentration ranging from 5-15 U/mg antibody and, optionally, wherein the antibody is added to the conjugation reaction at a concentration ranging from 5-20 mg/mL.

In a most preferred embodiment, the invention relates to the methods according to the invention, wherein the antibody is contacted with 2.5-12.5 molar equivalents of the linker; and/or wherein the microbial transglutaminase is added to the conjugation reaction at a concentration ranging from 5-15 U/mg antibody and, optionally, wherein the antibody is added to the conjugation reaction at a concentration ranging from 5-20 mg/mL.

In another preferred embodiment, the invention relates to the methods according to the invention, wherein the antibody is contacted with 2-20 molar equivalents of the linker; and/or wherein the microbial transglutaminase is added to the conjugation reaction at a concentration ranging from 5-15 U/mg antibody and, optionally, wherein the antibody is added to the conjugation reaction at a concentration ranging from 2.5-20 mg/mL.

It is to be noted that the specific reaction mixtures disclosed above may be freely combined with any of the buffer conditions disclosed herein. However, it is preferred that the specific components as defined above are mixed at a pH ranging from 6 to 8.

In a particular embodiment, the invention relates to an antibody-payload conjugate which has been produced with the method according to the invention.

That is, the invention further relates to an antibody-linker conjugate which has been generated with any of the aforementioned method steps.

In a particular embodiment, the invention relates to a pharmaceutical composition comprising the antibody-payload conjugate according to the invention and at least one pharmaceutically acceptable ingredient.

That is, the invention further relates to pharmaceutical compositions comprising the antibody-payload conjugate according to the invention.

The term “pharmaceutical composition” as used herein refers to any composition comprising a chemical substance or active ingredient which composition is intended for use in the medical cure, treatment, or prevention of disease and which is in such a form as to permit the active ingredient to be effective. In particular, a pharmaceutical composition does not contain excipients which are unacceptably toxic to a subject to which the composition is to be administered. The pharmaceutical compositions are sterile, i.e. aseptic and free from all living microorganisms and their spores. The pharmaceutical composition of the present invention is preferably liquid.

The type of payload that is comprised in the antibody-payload construct comprised in the pharmaceutical composition depends on the intended use of the pharmaceutical composition. In embodiments where the pharmaceutical composition is used for the treatment of a disease, the payload is preferably a drug. If the disease is a neoplastic disease, the payload is preferably a toxin. In embodiments where the pharmaceutical composition is used in diagnostics, the payload is preferably an imaging agent.

The pharmaceutical composition according to the invention may comprise an antibody-drug conjugate as disclosed herein. Pharmaceutical compositions comprising an antibody-drug conjugate are preferably used for the treatment of diseases.

The pharmaceutical composition according to the invention may comprise at least one pharmaceutically acceptable ingredient.

A pharmaceutically acceptable ingredient refers to an ingredient in a pharmaceutical formulation, other than an active ingredient, which is nontoxic to a subject. A pharmaceutically acceptable ingredient includes, but is not limited to, a buffer, excipient, stabilizer, or preservative.

Pharmaceutical formulations of the antibody-payload conjugates described herein are prepared by mixing such conjugates having the desired degree of purity with one or more optional pharmaceutically acceptable ingredients (Flemington's Pharmaceutical Sciences 16th edition, Osol, A. Ed. (1980)), in the form of lyophilized formulations or aqueous solutions. Pharmaceutically acceptable ingredients are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn protein complexes); and/or non-ionic surfactants such as polyethylene glycol (PEG). Exemplary pharmaceutically acceptable ingredients herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. For example, a sHASEGP may be combined with one or more additional glycosaminoglycanases such as chondroitinases.

In a particular embodiment, the invention relates to a pharmaceutical composition according to the invention comprising at least one additional therapeutically active agent.

That is, the pharmaceutical composition comprising the antibody-payload conjugate may comprise one or more additional therapeutically active agents. It is to be understood that the antibody-payload conjugates may be used in various therapeutic areas. As such, the additional therapeutically active agent in the pharmaceutical composition may vary depending on the use of the pharmaceutical composition.

In certain embodiments, a pharmaceutical composition comprising an antibody-payload conjugate according to the invention may be used in the treatment of cancer. In such embodiment, the pharmaceutical composition may comprise one or more additional anti-cancer drugs. The term “anti-cancer” drug is used herein to refer to one or a combination of drugs conventionally used to treat cancer.

For example, a pharmaceutical composition comprising an antibody-payload conjugate according to the invention may further comprise one or more chemotherapeutic agents. As used herein, the term “chemotherapeutic agent” or “chemotherapy agent” or “chemotherapeutic drug” refer to an agent that reduces, prevents, mitigates, limits, and/or delays the growth of metastases or neoplasms, or kills neoplastic cells directly by necrosis or apoptosis of neoplasms or any other mechanism, or that can be otherwise used, in a pharmaceutically-effective amount, to reduce, prevent, mitigate, limit, and/or delay the growth of metastases or neoplasms in a subject with neoplastic disease. Chemotherapeutic agents include, for example, fluoropyrimidines; pyrimidine nucleosides; purine nucleosides; anti-folates, platinum agents; anthracyclines/anthracenediones; epipodophyllotoxins; camptothecins; hormones; hormonal complexes; antihormonals; enzymes, proteins, peptides and polyclonal and/or monoclonal antibodies; vinca alkaloids; taxanes; epothilones; antimicrotubule agents; alkylating agents; antimetabolites; topoisomerase inhibitors; antivirals; and various other cytotoxic and cytostatic agents.

In a particular embodiment, the invention relates to the antibody-payload conjugate according to the invention, or the pharmaceutical composition according to the invention for use in therapy and/or diagnostics.

That is, the antibody-payload conjugate or the pharmaceutical composition according to the invention may be used in the treatment of a subject or in diagnosing a disease or condition in a subject. An individual or subject is preferably a mammal. Mammals include, but are not limited to, domesticated animals (e.g., cows, sheep, cats, dogs, and horses), primates (e.g., humans and non-human primates such as macaques), rabbits, and rodents (e.g., mice and rats). In certain embodiments, the individual or subject is a human. When an antibody-payload conjugate or a pharmaceutical composition comprising an antibody-payload conjugate according to the invention is used in therapy, it is preferred that the payload is a drug. When an antibody-payload conjugate or a pharmaceutical composition comprising an antibody-payload conjugate according to the invention is used in diagnostics, it is preferred that the linker comprises at least one imaging agent as payload.

In a particular embodiment, the invention relates to the antibody-payload conjugate according to the invention, or the pharmaceutical composition according to the invention for use in the treatment of a patient
suffering from,
being at risk of developing, and/or
being diagnosed for
a neoplastic disease, a neurological disease, an autoimmune disease, an inflammatory disease or an infectious disease.

In a particular embodiment, the invention relates to the antibody-payload conjugate according to the invention or the pharmaceutical composition according to the invention for use in treatment of a patient suffering from a neoplastic disease.

The patient suffering from cancer may be a patient who has not been previously treated with any anti-cancer therapy. However, the patient suffering from cancer may also be a patient who was refractory to a previous anti-cancer treatment.

The term “neoplastic disease” as used herein refers to a condition characterized by uncontrolled, abnormal growth of cells. Neoplastic diseases include cancer. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, kidney cancer, vulval cancer, thyroid cancer, hepatic carcinoma, skin cancer, melanoma, brain cancer, ovarian cancer, neuroblastoma, myeloma, various types of head and neck cancer, acute lymphoblastic leukemia, acute myeloid leukemia, Ewing sarcoma and peripheral neuroepithelioma. Preferred cancers include liver cancer, lymphoma, acute lymphoblastic leukemia, acute myeloid leukemia, Ewing sarcoma and peripheral neuroepithelioma.

That is, the antibody-payload conjugates according to the invention are preferably used for the treatment of cancer. As such, in certain embodiments, the antibody-payload conjugates according to invention comprise an antibody that specifically binds to an antigen that is present on a tumor cell. In certain embodiments, the antigen may be an antigen on the surface of a tumor cell. In certain embodiments, the antigen on the surface of the tumor cell may be internalized into the cell together with the antibody-payload conjugate upon binding of the antibody-payload conjugate to the antigen.

If the antibody-payload conjugate according to the invention is used in the treatment of cancer, it is preferred that the antibody-payload conjugate comprises at least one payload that has the potential to kill or inhibit the proliferation of the tumor cell to which the antibody-payload conjugate binds. In certain embodiments, the at least one payload exhibits its cytotoxic activity after the antibody-payload conjugate has been internalized into the tumor cell. In certain embodiments, the at least one payload is a toxin.

In a particular embodiment, the invention relates to the antibody-payload conjugate according to the invention or the pharmaceutical composition according to the invention for use in treatment of a patient suffering from an autoimmune disease.

In a particular embodiment, the invention relates to the antibody-payload conjugate according to the invention or the pharmaceutical composition according to the invention for use in treatment of a patient suffering from a bacterial infection or a viral infection.

In certain embodiments, the antibody-payload conjugate and/or the pharmaceutical composition according to the invention may be used in the treatment of B-cell-associated cancer.

Thus, in a particular embodiment, the invention relates to the antibody-payload conjugate or the pharmaceutical composition for use according to the invention, wherein the antibody-payload conjugate comprises Polatuzumab and wherein the neoplastic disease is a B-cell associated cancer.

For this, it is preferred that the antibody-payload conjugate comprises an anti-CD79b antibody as disclosed herein, preferably wherein the anti-CD79b antibody is internalized into a target cell upon binding to CD79b. In certain embodiments, the anti-CD79b antibody is Polatuzumab with a heavy chain as set forth in SEQ ID NO:71 and a light chain as set forth in SEQ ID NO:72. Further, it is preferred that the antibody-payload conjugate comprises at least one toxin.

In certain embodiments, the anti-CD79b antibody comprised in the antibody-payload conjugate or the pharmaceutical composition may be conjugated to any one of the linkers shown in FIGS. 1-40 or any one of the linkers disclosed herein.

A B-cell associated cancer may be any one selected from a group consisting of: high, intermediate and low grade lymphomas (including B cell lymphoma such as, for example, mucosa-associated lymphoid tissue B cell lymphoma and non-Hodgkin's lymphoma (NHL), mantle cell lymphoma, Burkitt's lymphoma, small lymphocytic lymphoma, marginal Zone lymphoma, diffuse large B cell lymphoma, follicular lymphoma, and Hodgkin's lymphoma and T cell lymphomas) and leukemias (including secondary leukemia, chronic lymphocytic leukemia (CLL), such as B cell leukemia (CD5+ B lymphocytes), myeloid leukemia, such as acute myeloid leukemia, chronic myeloid leukemia, lymphoid leukemia, such as acute lymphoblastic leukemia (ALL) and myelodysplasia), and other hematological and/or B cell—or T-cell-associated cancers, including cancers of additional hematopoietic cells, including polymorphonuclear leukocytes, such as basophils, eosinophils, neutrophils and monocytes, dendritic cells, platelets, erythrocytes and natural killer cells. Also included are cancerous B cell proliferative disorders selected from the following: lymphoma, non-Hodgkins lymphoma (NHL) aggressive NHL, relapsed aggressive NHL, relapsed indolent NHL, refractory NHL, refractory indolent NHL, chronic lymphocytic leukemia (CLL), Small lymphocytic lymphoma, leukemia, hairy cell leukemia (HCL), acute lymphocytic leukemia (ALL), and mantle cell lymphoma.

In a particular embodiment, the invention relates to the antibody-payload conjugate or the pharmaceutical composition for use according to the invention, wherein the B-cell associated cancer is non-Hodgkin lymphoma, in particular wherein the B-cell associated cancer is diffuse large B-cell lymphoma.

Further, the anti-CD79b antibody-payload conjugate and/or the pharmaceutical composition comprising an anti-CD79b antibody-payload conjugate may be used in conjunction with other therapies that are suitable for the treatment of B-cell-associated cancer.

Thus, in a particular embodiment the invention relates to the antibody-payload conjugate or the pharmaceutical composition for use according to the invention, wherein the antibody-payload conjugate or the pharmaceutical composition is administered in combination with bendamustine and/or rituximab.

It is to be understood that the antibody-payload conjugate or the pharmaceutical composition does not necessarily have to be administered at the same time as the additional therapeutic agent, such as bendamustine and/or rituximab. Instead the antibody-payload conjugate or the pharmaceutical composition may be administered with a different administration schedule and, consequently, on different days as other therapeutic agents that are used for the treatment of the same disease.

In certain embodiments, the antibody-payload conjugate and/or the pharmaceutical composition according to the invention may be used in the treatment of HER2-positive cancers.

That is, in a particular embodiment, the invention relates to the antibody-payload conjugate or the pharmaceutical composition for use according to the invention, wherein the antibody-payload conjugate comprises Trastuzumab and wherein the neoplastic disease is a HER2-positive cancer, in particular HER2-positive breast, gastric, ovarian or lung cancer.

For this, it is preferred that the antibody-payload conjugate comprises an anti-HER2/neu antibody as disclosed herein, preferably wherein the anti-HER2/neu antibody is internalized into a target cell upon binding to HER2/neu. In certain embodiments, the anti-HER2/neu antibody is Trastuzumab with a heavy chain as set forth in SEQ ID NO:73 and a light chain as set forth in SEQ ID NO:74. Further, it is preferred that the antibody-payload conjugate comprises at least one toxin.

In certain embodiments, the anti-HER2/neu antibody comprised in the antibody-payload conjugate or the pharmaceutical composition may be conjugated to any one of the linkers shown in FIGS. 1-40 or any one of the linkers disclosed herein.

A HER2-positive cancer, as used herein, may be, without limitation HER2-positive breast, gastric, ovarian or lung cancer. The skilled person is able to determine whether a cancer is a HER2-positive cancer. For example, tumor cells may be isolated in a biopsy and the presence of HER2/neu may be determined with any method known in the art.

Further, the anti-HER2/neu antibody-payload conjugate and/or the pharmaceutical composition comprising an anti-HER2/neu antibody-payload conjugate may be used in conjunction with other therapies that are suitable for the treatment of HER2-positive cancers.

Thus, in a particular embodiment, the invention relates to the antibody-payload conjugate or the pharmaceutical composition for use according to the invention, wherein the antibody-payload conjugate or the pharmaceutical composition is administered in combination with lapatinib, capecitabine and/or a taxane.

It is to be understood that the antibody-payload conjugate or the pharmaceutical composition does not necessarily have to be administered at the same time as the additional therapeutic agent, such as lapatinib, capecitabine and/or a taxane. Instead the antibody-payload conjugate or the pharmaceutical composition may be administered with a different administration schedule and, consequently, on different days as other therapeutic agents that are used for the treatment of the same disease.

In certain embodiments, the antibody-payload conjugate and/or the pharmaceutical composition according to the invention may be used in the treatment of Nectin-4-positive cancers.

That is, in a particular embodiment, the invention relates to the antibody-payload conjugate or the pharmaceutical composition for use according to the invention, wherein the antibody-payload conjugate comprises Enfortumab or an Enfortumab variant and wherein the neoplastic disease is a Nectin-4 positive cancer, in particular Nectin-4 positive pancreatic cancer, lung cancer, bladder cancer or breast cancer.

For this, it is preferred that the antibody-payload conjugate comprises an anti-Nectin-4 antibody as disclosed herein, preferably wherein the anti-Nectin-4 antibody is internalized into a target cell upon binding to Nectin-4. In certain embodiments, the anti-Nectin-4 antibody is Enfortumab with a heavy chain as set forth in SEQ ID NO:75 and a light chain as set forth in SEQ ID NO:76 or 77. Further, it is preferred that the antibody-payload conjugate comprises at least one toxin.

In certain embodiments, the anti-Nectin-4 antibody comprised in the antibody-payload conjugate or the pharmaceutical composition may be conjugated to any one of the linkers shown in FIGS. 1-40 or any one of the linkers disclosed herein.

A Nectin-4-positive cancer, as used herein, may be, without limitation Nectin-4-positive pancreatic cancer, lung cancer, bladder cancer or breast cancer. The skilled person is able do determine whether a cancer is a Nectin-4-positive cancer. For example, tumor cells may be isolated in a biopsy and the presence of Nectin-4 may be determined with any method known in the art.

The anti-Nectin-4 antibody-payload conjugate and/or the pharmaceutical composition comprising an anti-Nectin-4 antibody-payload conjugate according to the invention may be administered alone in patients who have previously received a PD-1 or PD-L1 inhibitor in combination with a platinum-based chemotherapeutic agent before or after surgery.

Further, the anti-Nectin-4 antibody-payload conjugate and/or the pharmaceutical composition comprising an anti-Nectin-4 antibody-payload conjugate may be used in conjunction with other therapies that are suitable for the treatment of Nectin-4-positive cancers.

Thus, in a particular embodiment, the invention relates to the antibody-payload conjugate or the pharmaceutical composition for use according to the invention, wherein the antibody-payload conjugate or the pharmaceutical composition is administered in combination with a platinum-based chemotherapeutic agent and/or Pembrolizumab.

It is to be understood that the antibody-payload conjugate or the pharmaceutical composition does not necessarily have to be administered at the same time as the additional therapeutic agent, such as the cisplatin-based chemotherapeutic agent and/or Pembrolizumab. Instead the antibody-payload conjugate or the pharmaceutical composition may be administered with a different schedule and, consequently, on different days as other therapeutic agents that are used for the treatment of the same disease

In a particular embodiment, the invention relates to a use of the antibody-payload conjugate according to the invention, or the pharmaceutical composition according to the invention for the manufacture of a medicament for the treatment of a patient
suffering from,
being at risk of developing, and/or
being diagnosed for
a neoplastic disease, neurological disease, an autoimmune disease, an inflammatory disease or an infectious disease.

In a particular embodiment, the invention relates to a method of treating or preventing a neoplastic disease, said method comprising administering to a patient in need thereof the antibody-payload conjugate according to the invention, or the pharmaceutical composition according to the invention.

In a particular embodiment, the invention relates to the antibody-payload conjugate according to the invention or the pharmaceutical composition according to the invention for use in pre-, intra- or post-operative imaging.

That is, the antibody-payload conjugate according to the invention may be used in medical imaging. For that, the antibody-payload conjugate may be visualized while binding to a specific target molecule, cell or tissue. Different techniques are known in the art to visualize particular payloads. For example, if the payload is a radionuclide, the molecules, cells, or tissues to which the antibody-payload conjugate binds may be visualized by PET or SPECT. If the payload is a fluorescent dye, the molecules, cells, or tissues to which the antibody-payload conjugate binds may be visualized by fluorescence imaging. In certain embodiments, the antibody-payload conjugate according to the invention comprises two different payloads, for example a radionuclide and a fluorescent dye. In this case, the molecule, cell or tissue to which the antibody-payload conjugate binds may be visualized using two different and/or complementary imaging techniques, for example PET/SPECT and fluorescence imaging.

The antibody-payload conjugate may be used for pre- intra- and/or post-operative imaging.

Pre-operative imaging encompasses all imaging techniques that may be performed before a surgery to make specific target molecules, cells or tissues visible when diagnosing a certain disease or condition and, optionally, to provide guidance for a surgery. Preoperative imaging may comprise a step of making a tumor visible by PET or SPECT before a surgery is performed by using an antibody-linker conjugate that comprises an antibody that specifically binds to an antigen on the tumor and is conjugated to a payload that comprises a radionuclide.

Intra-operative imaging encompasses all imaging techniques that may be performed during a surgery to make specific target molecules, cells or tissues visible and thus provide guidance for the surgeon. In certain embodiments, an antibody-linker conjugate comprising a near-infrared fluorescent dye may be used to visualize a tumor during surgery by near-infrared fluorescent imaging. Intraoperative imaging allows the surgeon to identify specific tissues, for example tumor tissue, during surgery and thus may allow complete removal of tumor tissue.

Post-operative imaging encompasses all imaging techniques that may be performed after a surgery to make specific target molecules, cells or tissues visible and to evaluate the result of the surgery. Post-operative imaging may be performed similarly as pre-operative surgery.

In particular, the invention relates to antibody-payload conjugates comprising two or more different payloads. For example, the antibody-linker conjugate may comprise a radionuclide and a near-infrared fluorescent dye. Such an antibody-payload conjugate may be used for imaging by PET/SPECT and near-infrared fluorescent imaging. The advantage of such an antibody is that it may be used to visualize the target tissue, for example a tumor before and after a surgery by PET or SPECT. At the same time, the tumor may be visualized during the surgery by near-fluorescent infrared imaging.

In a particular embodiment, the invention relates to the antibody-payload conjugate or the pharmaceutical composition according to the invention, in particular wherein the antibody-payload conjugate comprises two payloads, for use in intraoperative imaging-guided cancer surgery.

As mentioned above, the antibody-payload conjugate of the invention may be used to visualize a target molecule, cell or tissue and to guide a surgeon or robot during a surgery. That is, the antibody-payload conjugate may be used to visualize tumor tissue during a surgery, for example by near-infrared imaging and to allow complete removal of the tumor tissue.

The antibody-payload conjugate or the pharmaceutical composition according to the invention may be administered to the human or animal subject in an amount or dosage that efficiently treats a disease or is sufficient for diagnostic purposes.

The antibody-payload conjugate or the pharmaceutical composition according to the invention may be administered by any suitable means, including parenteral, intrapulmonary, and intranasal, and, if desired for local treatment, intralesional, intrauterine or intravesical administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The antibody-payload conjugate or the pharmaceutical composition according to the invention may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody-payload conjugate or the pharmaceutical composition according to the invention need not be, but is optionally formulated with one or more agents currently used to prevent or treat the disorder in question. The effective amount of such other agents depends on the amount of antibody-payload conjugate present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of the antibody-payload conjugate or the pharmaceutical composition according to the invention (when used alone or in combination with one or more other additional therapeutic agents) will depend on the type of disease to be treated, the type of antibody-payload conjugate, the severity and course of the disease, whether the antibody-linker conjugate is administered for preventive or therapeutic purposes, previous therapy, the patient's clinical history and response to the antibody-linker conjugate, and the discretion of the attending physician. The antibody-payload conjugate or the pharmaceutical composition according to the invention is suitably administered to the patient at one time or over a series of treatments.

### BRIEF DESCRIPTION OF THE DRAWINGS

FIG. 1: Chemical structure of the linker Ac-RKAA-PABC-(MMAE)₂.

FIG. 2: Chemical structure of the linker Ac-RKAA-PABC-PABC-(MMAE)₂.

FIG. 3: Chemical structure of the linker MMAE-PABC-AA-C2-RKAA-PABC-MMAE.

FIG. 4: Chemical structure of the linker Ac-RKAA-PABC-(Exa)₂.

FIG. 5: Chemical structure of the linker Ac-ARK-PABC-(Exa)₂.

FIG. 6: Chemical structure of the linker Ac-RKARA-PABC-(Exa)₂.

FIG. 7: Chemical structure of the linker Ac-RKAAAA-PABC-(Exa)₂.

FIG. 8: Chemical structure of the linker Ac-RKAAAAAA-PABC-(Exa)₂.

FIG. 9: Chemical structure of the linker Ac-RKAASGSG-PABC-(Exa)₂.

FIG. 10: Chemical structure of the linker Ac-RKHA-PABC-(Exa)₂.

FIG. 11: Chemical structure of the linker Ac-RKHAAA-PABC-(Exa)₂.

FIG. 12: Chemical structure of the linker Ac-HKA-PABC-(Exa)₂.

FIG. 13: Chemical structure of the linker Ac-RKAA-PABC-(G-Exa)₂.

FIG. 14: Chemical structure of the linker Exa-PABC-AA-C2-RKAA-PABC-Exa.

FIG. 15: Chemical structure of the linker GGR-PABC-(Exa)₂.

FIG. 16: Chemical structure of the linker GGRG-PABC-(G-Exa)₂.

FIG. 17: Chemical structure of the linker Ac-RKAA-PABC-(G-Exa′)₂.

FIG. 18: Chemical structure of the linker GGRG-PABC-(G-Exa′)₂.

FIG. 19: Chemical structure of the linker Ac-E(A-PABC-MMAE)ARKAA-PABC-(MMAE)₂.

FIG. 20: Chemical structure of the linker (MMAE)₂-PABC-AA-C2-RKAA-PABC-(MMAE)₂.

FIG. 21: Chemical structure of the linker Exa-PABC-AA-C2-RKAA-PABC-(MMAE)₂.

FIG. 22: Chemical structure of the linker May-C5-RKAE(A-PABC-MMAE)A-EDA-Cortisol.

FIG. 23: Chemical structure of the linker RhKAA-PABC-(MMAE)₂.

FIG. 24: Chemical structure of the linker NH₂-C5-GRG-PABC-(MMAE)₂.

FIG. 25: Chemical structure of the linker RKVCit-PABC-PABC-(MMAE)₂.

FIG. 26: Chemical structure of the linker Exa-PABC-RA-C3-RKAR-PABC-MMAE.

FIG. 27: Chemical structure of the linker Cryptophycin-AA-C2-RKVA-Cryptophycin.

FIG. 28: Chemical structure of the linker KAR-PABC-EDA-BHMC-(MMAF)₂.

FIG. 29: Chemical structure of the linker RK-E(PEG12-FA)AA-PABC-MMAE.

FIG. 30: Chemical structure of the linker E(AA-AM-Dxd)RKAA-AM-Dxd.

FIG. 31: Chemical structure of the linker cRGD-PEG4-RKAH-PABC-EDA-PNU.

FIG. 32: Chemical structure of the linker Biotin-RKAN-PABQ-Rifalog.

FIG. 33: Chemical structure of the linker May-RKGGFG-PABC-AMP-AE.

FIG. 34: Chemical structure of the linker Resiquimod-CitV-C2-RKGP-STING.

FIG. 35: Chemical structure of the linker C(May)-RKAA-AM-May.

FIG. 36: Chemical structure of the linker K(SMCC-May)-RKAA-(ValCit-PABC-MMAE)₂.

FIG. 37: Chemical structure of the linker S(Glyco)-RKAA-(AA-PABC-MMAE)₂.

FIG. 38: Chemical structure of the linker Exa-gluc-C3-RK-C3-gluc-Exa.

FIG. 39: Chemical structure of the linker D(AA-AM-Dxd)-D(AA-AM-Dxd)-RKAA-AM-Dxd.

FIG. 40: Chemical structure of the linker (E(AA-PABC-G-Dxd))₂RKVCit-PABC-PABC-(G-Dxd)₂.

FIG. 41: Chemical structure of the linker NH₂-PEG2-PABC-(MMAE)₂.

FIG. 42: Chemical structure of the linker MMAE-PABC-AA-C2-KAR-PABC-MMAE.

FIG. 43: Chemical structure of the linker RKN(PABC-MMAE)A-PABC-MMAE.

FIG. 44: Anti-tumor efficacy of two DAR4 linkers of the invention in comparison to the benchmark antibody Enfortumab vedotin in a Nectin-4 positive solid tumor model.

FIG. 45: Anti-tumor efficacy of two DAR4 linkers of the invention in a CD79b-positive liquid tumor model.

### EXAMPLES

### General Methods

The antibody Trastuzumab was commercially available (Herceptin®, Roche, bought from a pharmacy), as well as all the peptides-linkers and linker-payloads (custom synthesized by LifeTein and Levena Biopharma, respectively). DNA constructs encoding Polatuzumab with heavy and light chain consisting of the sequences of SEQ ID NOs: 71 and 72 as well as Enfortumab with heavy and light chain consisting of the sequences of SEQ ID NO: 75 and 76 were transiently transfected into suspension-adapted CHO-K1 cells and expressed in serum-free/animal component-free media. The proteins were purified from the supernatants by Protein A affinity chromatography (Mab Select Sure column; GE Healthcare).

Conjugation reactions were performed by mixing 5 mg/ml of native, glycosylated monoclonal antibody, microbial transglutaminase (MTG, Zedira) at a concentration of 5-10 U/mg, and 5-20 molar equivalents of the indicated linker-payload, in Tris 50 mM pH 7.6, or BisTris pH 6.0-6.8 for 24 hours at 37° C. in a rotating thermomixer. Conjugation efficiency was assessed by LC-MS, or RPLC, under DTT reduced conditions. Reduction of samples was achieved by incubation of the samples for 15 min at 37° C. in 50 mM DTT (final) and 50 mM Tris buffer.

LCMS: after reduction, samples were analyzed on a Xevo G2-XS QTOF (Waters) coupled to an Acquity UPLC H-Class System (Waters) and an ACQUITY UPLC BEH C18 Column. Conjugation efficiency (CE) was calculated from deconvoluted spectra and presented in %. Intensities resulting from both glycoforms (G1F and G0F) were taken into account for the calculation, according to the formula:

$$CE \% = \frac{\sum ( {( Int ( G 0 F + G 1 F ) )}_{cj} )}{\sum {( Int ( G 0 F + G 1 F ) )}_{cj , ncj}}$$

With cj=conjugated and ncj=non-conjugated

RPLC: after reduction, samples were analyzed on a UHPLC Dionex UltiMate 3000 (Thermo Fisher) using BioResolve RP mAb Polyphenyl column. Conjugation efficiency (CE) was calculated using relative peak area extracted from the RPLC chromatogram according to the formula and presented in %:

$$CE \% = \frac{\sum ( {( Int ( relative peak area ) )}_{cj} )}{\sum {( Int ( relative peak area ) )}_{cj , ncj}}$$

With cj=conjugated and ncj=non-conjugated

### Example 1: Conjugation of Various MMAE Linker-Payload Constructs for Preparation of Trastuzumab DAR 4 ADC

### Method

Reaction conditions: 5 mg/ml of native, pharmacy-bought, fully glycosylated, Trastuzumab antibody (Herceptin® bought at Pharmacy), MTG at a concentration of 5 U/mg, and 5 molar equivalents of the indicated linker-payload, in Tris 50 mM pH 7.6 for 24 hours at 37° C. in a rotating thermomixer. Conjugation efficiency was assessed by LCMS as described above.

### Results

Surprisingly, excellent conjugation efficiencies were obtained using various MMAE-linker-payload constructs according to this invention (Table 1), to native, fully glycosylated Trastuzumab, for preparation of DAR 4 ADCs.

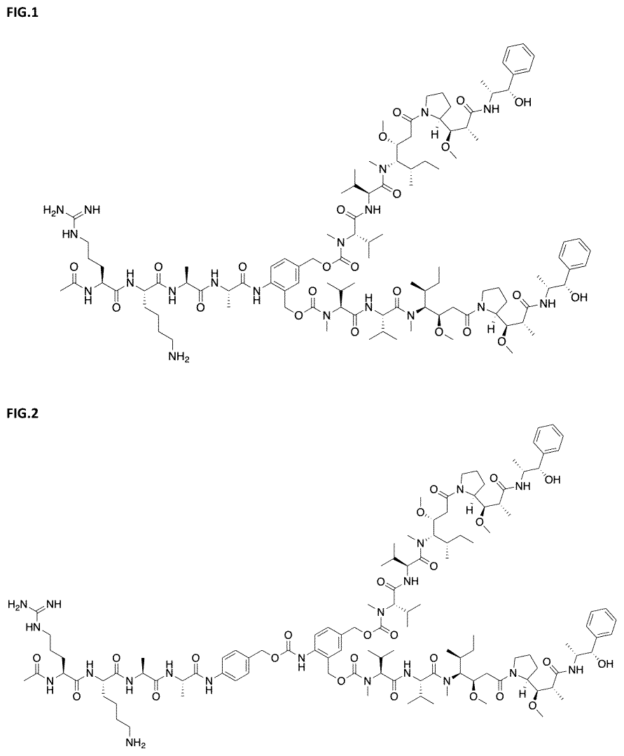

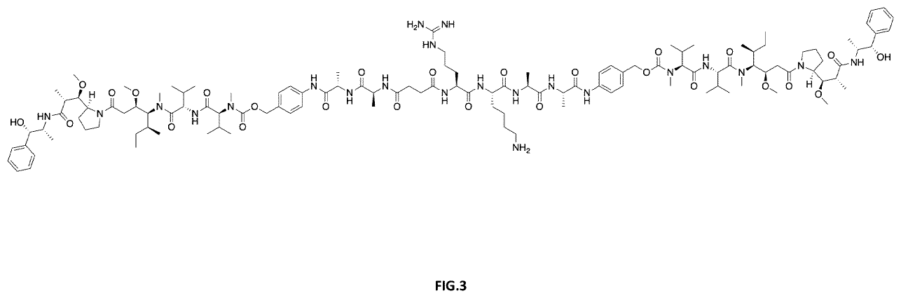

TABLE 1
Conjugation efficiencies (%) obtained using various MMAE linker-payload constructs for
preparation of Trastuzumab DAR 4 ADC

| | Conjugation efficiency (%) |
|---|---|
| Linker-payload with MMAE for preparation of DAR 4 ADCs | to Trastuzumab |
| RKAA-PABC-(MMAE)₂ (SEQ ID NO: 1; FIG. 1) | 90% |
| RKAA-PABC-PABC-(MMAE)₂ (SEQ ID NO: 1; FIG. 2) | 90% |
| MMAE-PABC-AA-C2-RKAA-PABC-MMAE (SEQ ID NO: 1; FIG. 3) | 95% |

### Example 2: Conjugation of Various MMAE Linker-Payload Constructs for Preparation of DAR 4 ADC with Two Other Different Antibodies

To demonstrate the versatility of the invention, various MMAE linker-payloads were used to generate DAR 4 MMAE ADCs using two different parental antibodies: Polatuzumab and Enfortumab.

### Method

Conjugation reactions were performed by mixing 5 mg/ml of the indicated native, glycosylated antibody, MTG at a concentration of 6-7.5 U/mg, and 5 molar equivalents of the indicated linker-payload, in Tris 50 mM pH 7.6 for 24 hours at 37° C. in a rotating thermomixer. Conjugation efficiency was assessed by LCMS as described above.

### Results

Surprisingly, excellent conjugation efficiencies were obtained using various MMAE-linker-payload constructs according to this invention for conjugation to native, fully glycosylated Polatuzumab (Table 2a) and Enfortumab (Table 2b) antibodies, respectively, for preparation of DAR 4 ADCs.

TABLE 2a
Conjugation efficiencies (%) obtained using various MMAE linker-payload constructs for
preparation of Polatuzumab DAR 4 ADCs

| Linker-payload with MMAE for preparation of DAR 4 | Conjugation efficiency (%) |
|---|---|
| Polatuzumab ADCs | to Polatuzumab |
| RKAA-PABC-(MMAE)₂ (SEQ ID NO: 1; FIG. 1) | 91% |
| RKAA-PABC-PABC-(MMAE)₂ (SEQ ID NO: 1; FIG. 2) | 89% |
| MMAE-PABC-AA-C2-RKAA-PABC-MMAE (SEQ ID NO: 1; FIG. 3) | 96% |

TABLE 2b
Conjugation efficiencies (%) obtained using various MMAE linker-payload constructs for
preparation of Enfortumab DAR 4 ADCs

| Linker-payload with MMAE for preparation of DAR4 | Conjugation efficiency (%) |
|---|---|
| Enfortumab ADCs | to Enfortumab |
| RKAA-PABC-(MMAE)₂ (SEQ ID NO: 1; FIG. 1) | 90% |
| RKAA-PABC-PABC-(MMAE)₂ (SEQ ID NO: 1; FIG. 2) | 92% |
| MMAE-PABC-AA-C2-RKAA-PABC-MMAE (SEQ ID NO: 1; FIG. 3) | 96% |

### Example 3: Conjugation of Various Exatecan Linker-Payload Constructs for Preparation of Trastuzumab DAR 4 ADC

To demonstrate the broad applicability of the invention, linker-payloads with Exatecan (a Topoisomerase I inhibitor) were designed and evaluated for the preparation of Trastuzumab DAR 4 Exatecan ADCs.

### Method

Reaction conditions: 5 mg/ml of native, glycosylated Trastuzumab antibody, MTG at a concentration of 10 U/mg, and 7.5-12.5 molar equivalents of the indicated linker-payload, in BisTris pH 6.6 for 24 hours at 37° C. in a rotating thermomixer. Conjugation efficiency was assessed by LCMS or RPLC as described above.

### Results

Surprisingly, excellent conjugation efficiencies were obtained using various Exatecan linker-payload constructs according to this invention (Table 3), to native, fully glycosylated Trastuzumab, for preparation of DAR 4 ADCs.

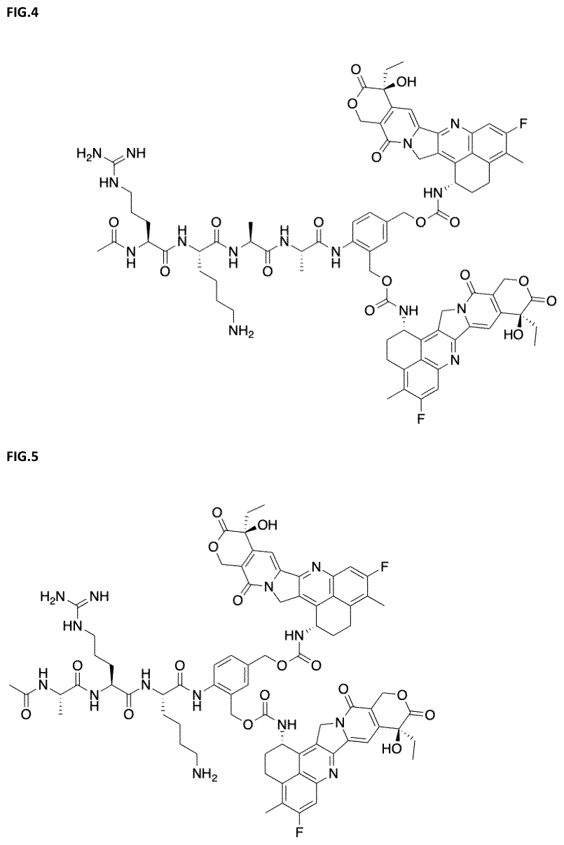

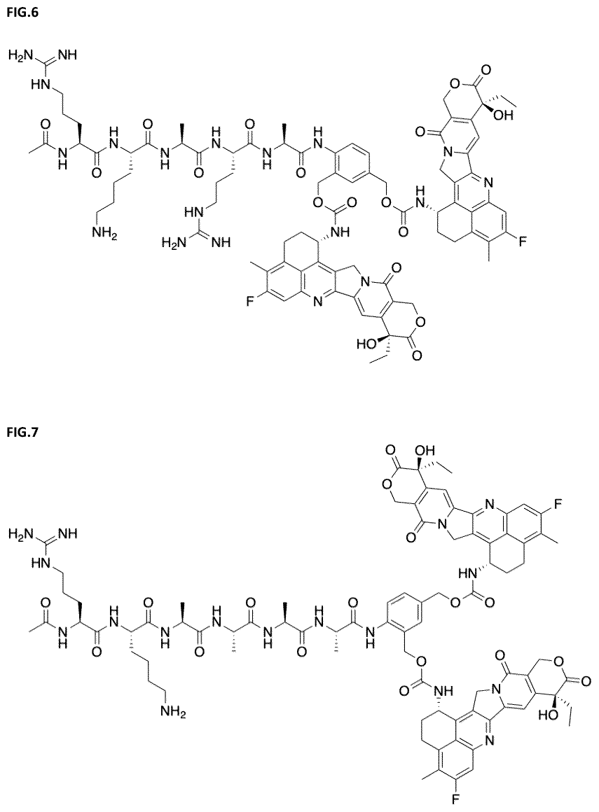

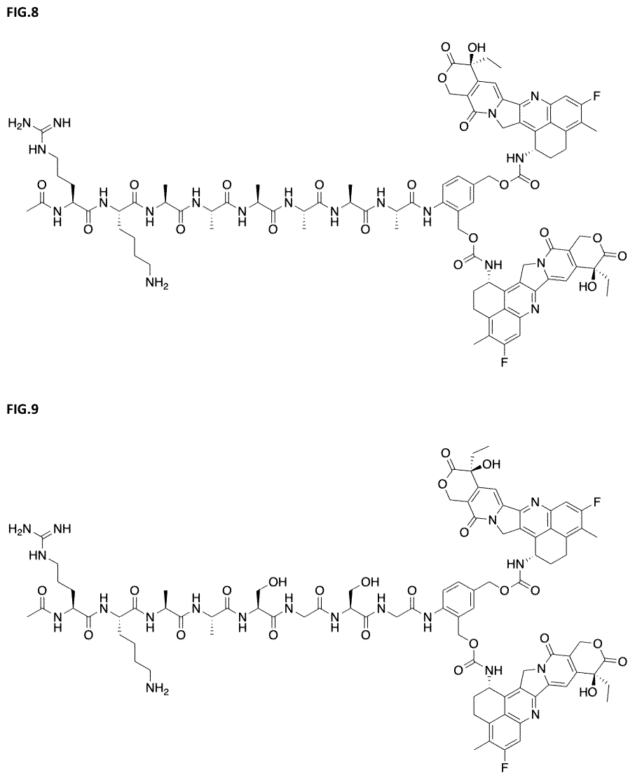

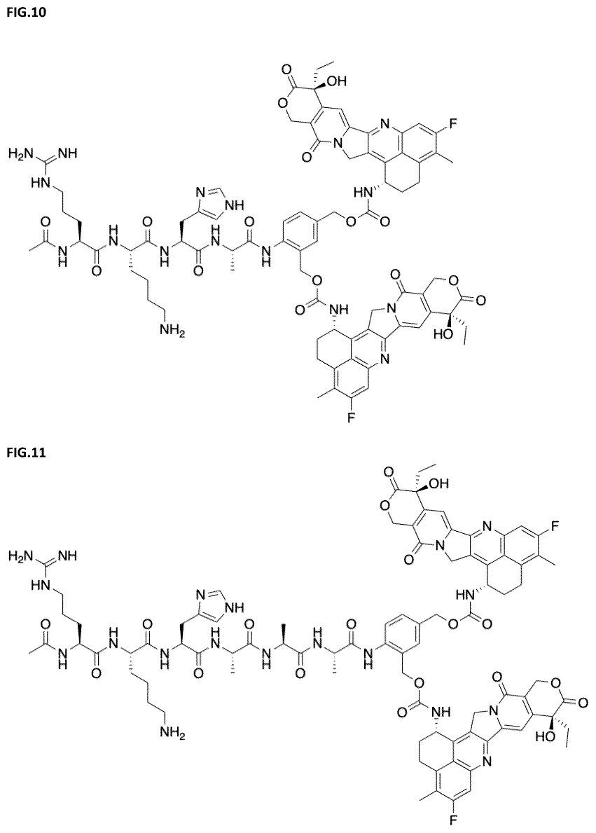

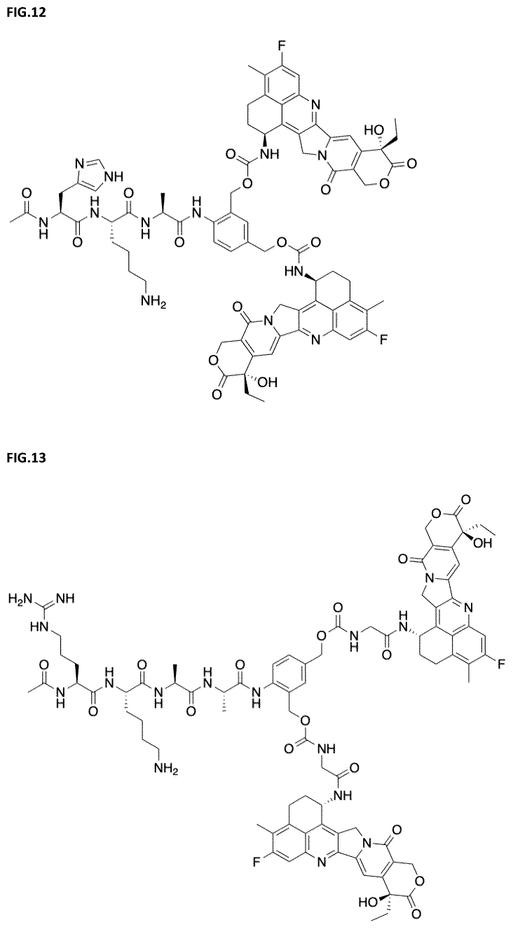

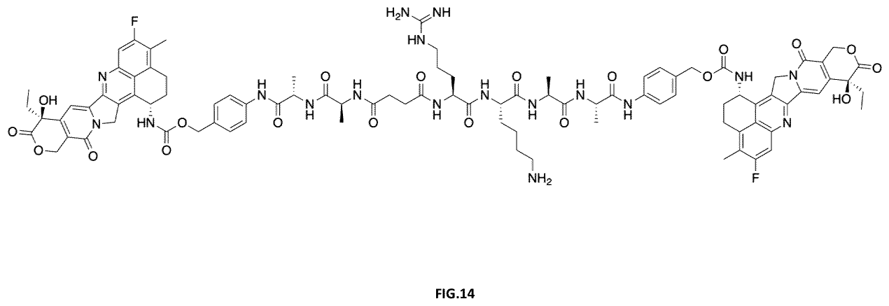

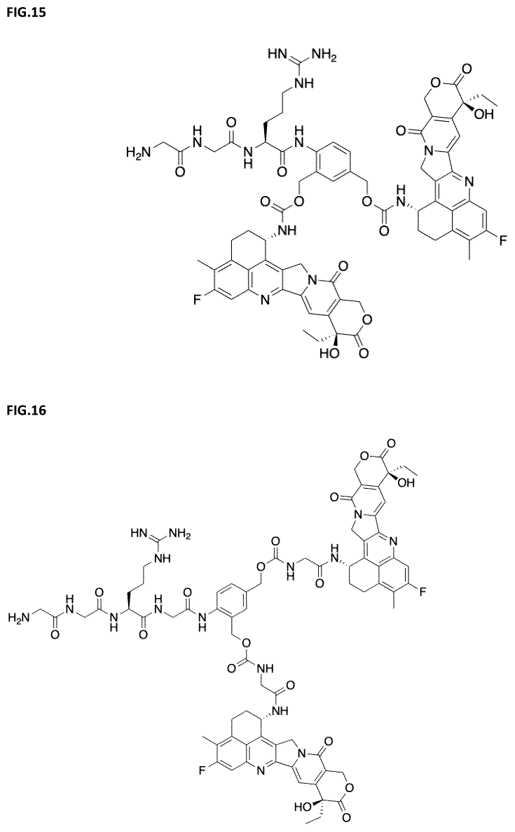

TABLE 3
Conjugation efficiencies (%) obtained using various Exatecan linker-payload
constructs for preparation of Trastuzumab DAR 4 ADCs

| Linker-payload with Exatecan for preparation | Conjugation efficiency (%) |
|---|---|
| of DAR4 ADCs | to Trastuzumab |
| RKAA-PABC-(Exa)₂ (SEQ ID NO: 1; FIG. 4) | 92% |
| ARK-PABC-(Exa)₂ (SEQ ID NO: 2; FIG. 5) | 91% |
| RKARA-PABC-(Exa)₂ (SEQ ID NO: 3; FIG. 6) | 90% |
| RKAAAA-PABC-(Exa)₂ (SEQ ID NO: 4; FIG. 7) | 99% |
| RKAAAAAA PABC-(Exa)₂ (SEQ ID NO: 5; FIG. 8) | 95% |
| RKAASGSG-PABC-(Exa)₂ (SEQ ID NO: 6; FIG. 9) | 92% |
| RKHA-PABC-(Exa)₂ (SEQ ID NO: 7; FIG. 10) | 94% |
| RKHAAA-PABC-(Exa)₂ (SEQ ID NO: 8; FIG. 11) | 98% |
| HKA-PABC-(Exa)₂ (SEQ ID NO: 13; FIG. 12) | 80% |
| RKAA PABC-(G-Exa)₂ (SEQ ID NO: 1; FIG. 13) | 91% |
| Exa-PABC-AA-C2-RKAA-PABC-Exa (SEQ ID NO: 1; FIG. 14) | 88% |
| GGR-PABC-(Exa)₂ (SEQ ID NO: 9; FIG. 15) | 98% |
| GGRG-PABC-(G-Exa)₂ (SEQ ID NO: 10; FIG. 16) | 99% |

### Example 4: Conjugation of Various Exatecan Analog Linker-Payload Constructs for Preparation of DAR 4 ADC to Trastuzumab

To demonstrate the high tolerability of the reaction, linker-payload constructs for the preparation of DAR 4 ADCs were assessed with an analog of Exatecan described in Li et al 2019 (compound 10). This analog is called Exatecan′ (or Exa′) in the frame of Example 4 and Example 5.

### Method

Reaction conditions: 5 mg/ml of native, glycosylated Trastuzumab antibody, MTG at a concentration of 10 U/mg, and 7.5 molar equivalents of the indicated linker-payload, in BisTris pH 6.6 for 24 hours at 37° C. in a rotating thermomixer. Conjugation efficiency was assessed by LCMS as described above.

### Results

Surprisingly, excellent conjugation efficiencies were obtained using various Exatecan′ linker-payload constructs according to this invention (Table 4), to native, fully glycosylated Trastuzumab, for preparation of DAR 4 ADCs.

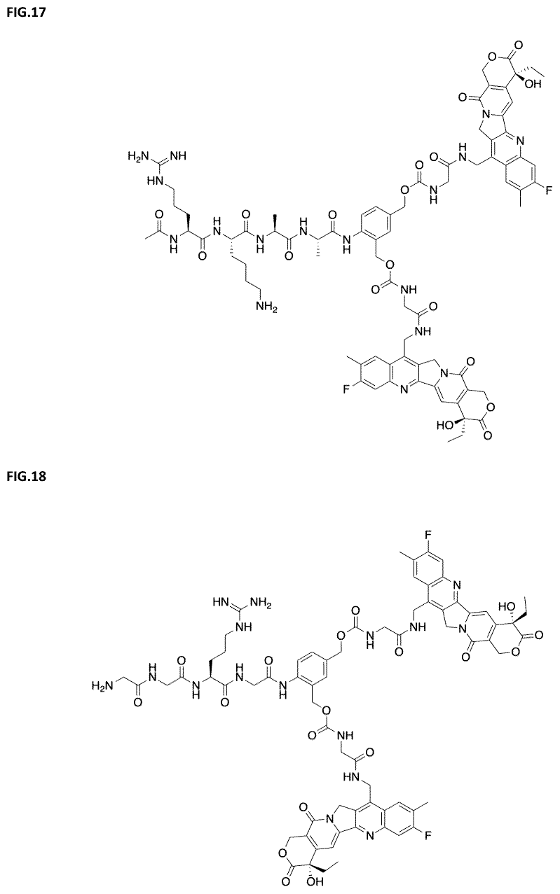

TABLE 4
Conjugation efficiencies (%) obtained using various Exatecan′ linker-payload
constructs for preparation of Trastuzumab DAR 4 ADCs

| Linker-payload with Exatecan analog for | Conjugation efficiency (%) |
|---|---|
| ADCspreparation of DAR4 | to Trastuzumab |
| RKAA-PABC-(G-Exa′)₂ (SEQ ID NO: 1; FIG. 17) | 99% |
| GGRG-PABC-(G-Exa′)₂ (SEQ ID NO: 10; FIG. 18) | 99% |

### Example 5: Conjugation of Various Exatecan (or Exatecan-Analog) Linker-Payload Constructs for Preparation of DAR 4 ADC with Two Other Different Antibodies

To demonstrate the versatility of the reaction, various Exatecan (Exa) or Exatecan-analog (Exa′) linker-payload constructs were conjugated to two additional antibodies: Polatuzumab and Enfortumab to generate Polatuzumab DAR 4 Exa or Polatuzumab DAR 4 Exa′ and Enfortumab DAR 4 Exa or Enfortumab DAR 4 Exa′ ADCs.

### Method

Conjugation reactions were performed by mixing 5 mg/ml of the indicated native, glycosylated antibody, MTG at a concentration of 7.5 U/mg, and 10 molar equivalents of the indicated linker-payload, in Tris 50 mM pH 7.6 for 24 hours at 37° C. in a rotating thermomixer. Conjugation efficiency was assessed by RPLC as described above.

### Results

Surprisingly, excellent conjugation efficiencies were obtained using various Exatecan (or analog) linker-payload constructs according to this invention for conjugation to native, fully glycosylated Polatuzumab (Table 5a) and Enfortumab (Table 5b) antibodies, respectively, for preparation of DAR 4 ADCs.

TABLE 5a
Conjugation efficiencies (%) obtained using various Exatecan (Exa) or Exatecan-analog
(Exa′) linker-payload constructs to generate Polatuzumab DAR 4 ADCs

| Linker-payload with Exatecan (or analog) for preparation | Conjugation efficiency (%) |
|---|---|
| of Polatuzumab DAR 4 ADCs | to Polatuzumab |
| RKAA-PABC-(G-Exa)₂ (SEQ ID NO: 1; FIG. 13) | 95% |
| RKAA-PABC-(G-Exa′)₂ (SEQ ID NO: 1; FIG. 17) | 100% |
| GGRG-PABC-(G-Exa)₂ (SEQ ID NO: 10; FIG. 16) | 98% |
| GGRG-PABC-(G-Exa′)₂ (SEQ ID NO: 10; FIG. 18) | 100% |

TABLE 5b
Conjugation efficiencies (%) obtained using various Exatecan (Exa) or Exatecan-analog
(Exa′) linker-payload constructs to generate Enfortumab DAR 4 ADCs

| Linker-payload with Exatecan (or analog) for preparation | Conjugation efficiency (%) |
|---|---|
| of Enfortumab DAR 4 ADCs | to Enfortumab |
| RKAA-PABC-(G-Exa)₂ (SEQ ID NO: 1; FIG. 13) | 95% |
| RKAA-PABC-(G-Exa′)₂ (SEQ ID NO: 1; FIG. 17) | 98% |
| GGRG-PABC-(G-Exa)₂ (SEQ ID NO: 10; FIG. 16) | 100% |
| GGRG-PABC-(G-Exa′)₂ (SEQ ID NO: 10; FIG. 18) | 100% |

### Example 6: Conjugation of Various MMAE Linker-Payload Constructs for Preparation of Trastuzumab DAR 6 or DAR 8 ADC

To demonstrate the wide applicability of the invention, we aimed at generating DAR>4 ADCs. For that, various MMAE linker-payload constructs were designed and evaluated to generate DAR 6 or DAR 8 ADCs.

### Method

Reaction conditions: 5 mg/ml of native, glycosylated Trastuzumab antibody, MTG at a concentration of 8 U/mg, and 5 molar equivalents of the indicated linker-payload, in BisTris 50 mM pH 7.5 for 22 hours at 37° C. in a rotating thermomixer. Conjugation efficiency was assessed by LCMS as described above.

### Results

Strikingly, structures according to this invention containing 3 or 4 MMAE moiety per linker-payload construct led to good or excellent conjugation efficiencies (Table 6) to native, fully glycosylated Trastuzumab resulting in Trastuzumab-DAR 6 MMAE or DAR 8 MMAE ADCs. The conjugation efficiencies were >67% surprisingly significantly higher as historically shown for a lysine-based linker yielding a DAR2 ADC (see Example 5 of WO 2015/191883, where conjugation efficiency of only 40% could be achieved).

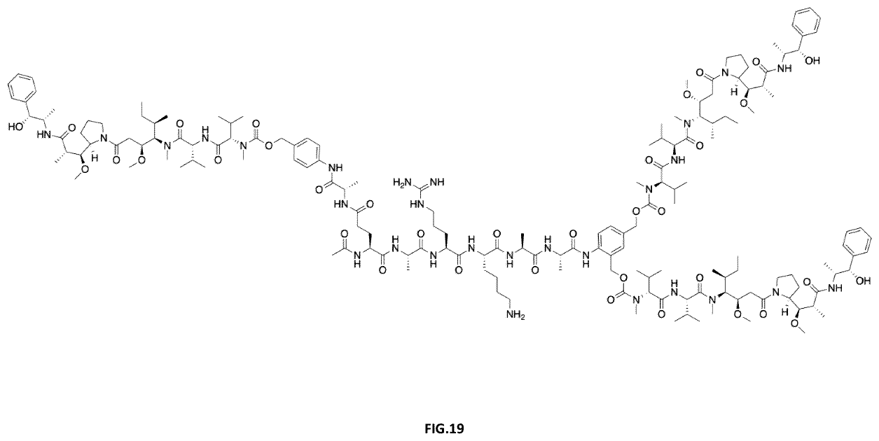

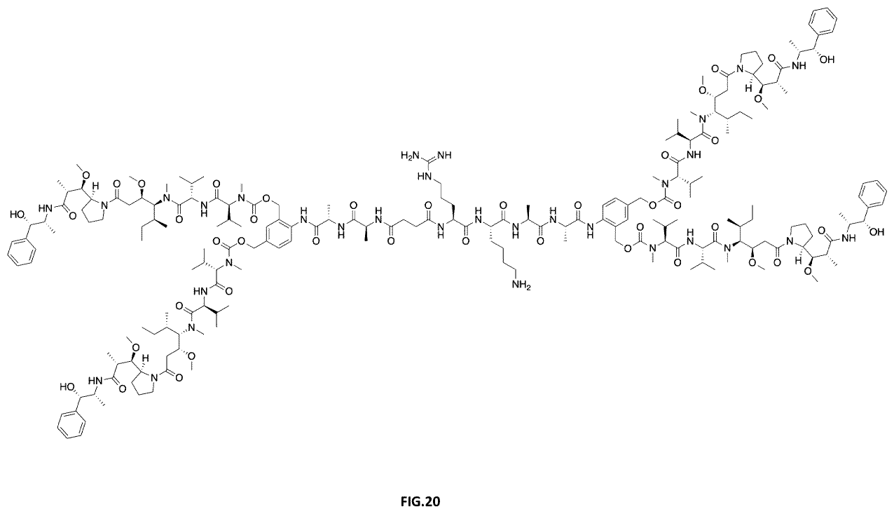

TABLE 6
Conjugation efficiencies (%) obtained using various MMAE linker-payload constructs to
generate Trastuzumab DAR 6-MMAE or DAR 8-MMAE ADCs

| Linker-payload with MMAE for preparation of | Conjugation efficiency (%) |
|---|---|
| DAR 6 or DAR 8 ADCs | to rastuzumab |
| E(A-PABC-MMAE)-ARKAA-PABC-(MMAE)₂ (SEQ ID NO: 11; FIG. 19)) | 99% |
| (MMAE)₂-PABC-AA-RKAA-PABC-(MMAE)₂ (SEQ ID NO: 1; FIG. 20) | 67% |

### Example 7: Conjugation of MMAE-Exatecan Linker-Payload Construct for Preparation of DAR 6 Trastuzumab Dual-Payload ADC Comprising 4 MMAE and 2 Exatecan

To demonstrate that the invention may also include a linker-payload comprising different drug types in one linker-payload, a structure containing two different drugs (MMAE and Exatecan) was designed and conjugated to Trastuzumab resulting in a DAR6 Trastuzumab-dual-payload ADC comprising 4 MMAE and 2 Exatecan.

### Method

Reaction conditions: 5 mg/ml of native, glycosylated Trastuzumab antibody, MTG at a concentration of 8 U/mg, and 5 molar equivalents of the indicated linker-payload, in BisTris 50 mM pH 7.5 for 22 hours at 37° C. in a rotating thermomixer. Conjugation efficiency was assessed by LCMS as described above.

### Results

Surprisingly, a structure according to this invention containing two different drug types per linker-payload construct (i.e. 2×MMAE and 1x Exatecan) led to a good conjugation efficiency (Table 7) to native, fully glycosylated Trastuzumab, resulting in a DAR6 Trastuzumab-dual-payload ADC comprising 4 MMAE and 2 Exatecan.

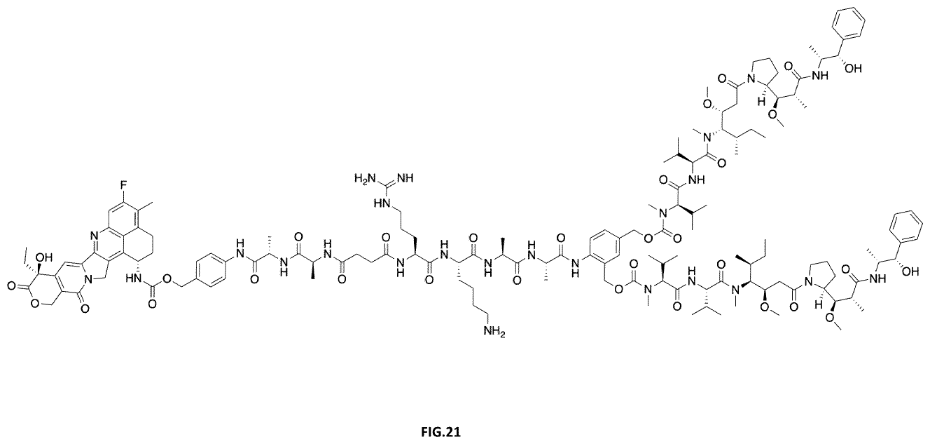

TABLE 7
Conjugation efficiency (%) obtained using MMAE-Exatecan linker-payload to generate a DAR6
Trastuzumab dual-payload ADC comprising 4 MMAE and 2 Exatecan

| Linker-payload containing dual-payload MMAE-Exatecan for | Conjugation efficiency (%) |
|---|---|
| preparation of DAR 6 ADC comprising 4 MMAE and 2 Exatecan | to Trastuzumab |
| Exa-PABC-AA-RKAA-PABC-(MMAE)₂ (SEQ ID NO: 1; FIG. 21) | 79% |

### Example 8: Conjugation of MMAE-Maytansine-Cortisol Linker-Payload Construct for Preparation of DAR6 Trastuzumab Tri-Payload ADC Comprising 2 MMAE, 2 Maytansine and 2 Cortisol

To demonstrate that the invention may also include three different drug types and payload classes on one linker-payload construct, a structure containing three different payloads (MMAE, Maytansine and Cortisol CS) was designed and conjugated to Trastuzumab resulting in DAR6 Trastuzumab tri-payload ADC comprising 2 MMAE, 2 Maytansine and 2 Cortisol.

### Method

Reaction conditions: 5 mg/ml of native, glycosylated Trastuzumab antibody, MTG at a concentration of 8 U/mg, and 5 molar equivalents of the indicated linker-payload, in BisTris 50 mM pH 7.5 for 22 hours at 37° C. in a rotating thermomixer. Conjugation efficiency was assessed by LCMS as described above.

### Results

Surprisingly, a structure according to this invention containing three different payload types per linker-payload construct (i.e. 1×MMAE, 1×May and 1×CS) led to an excellent conjugation efficiency (Table 8) to native, fully glycosylated Trastuzumab, resulting in a in DAR6 Trastuzumab tri-payload ADC comprising 2 MMAE, 2 Maytansine and 2 Cortisol.

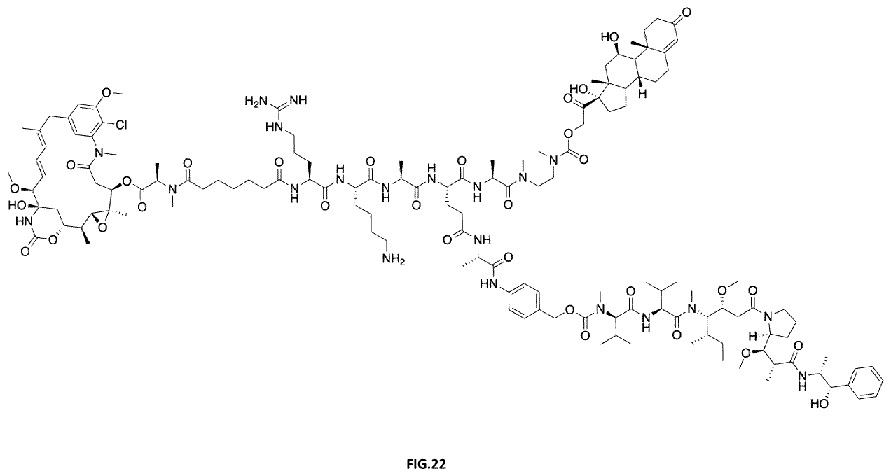

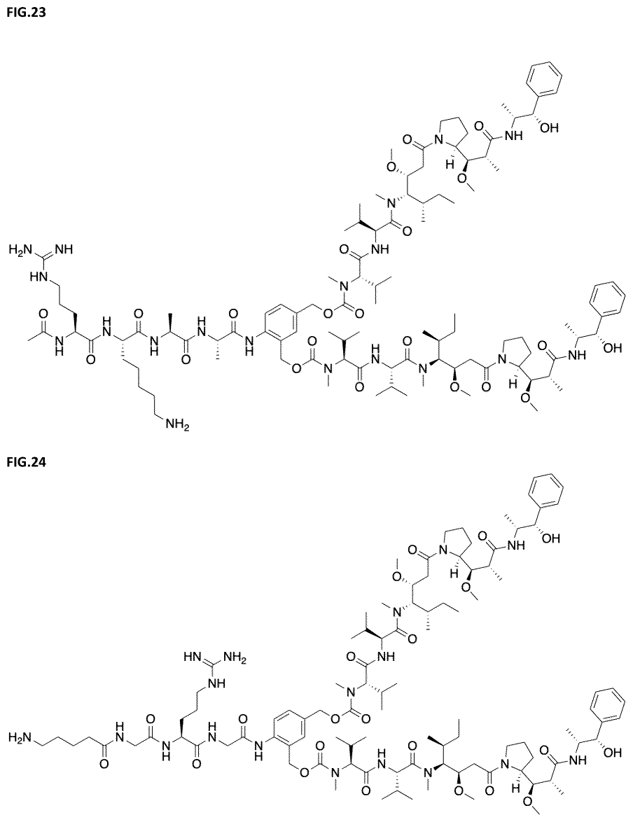

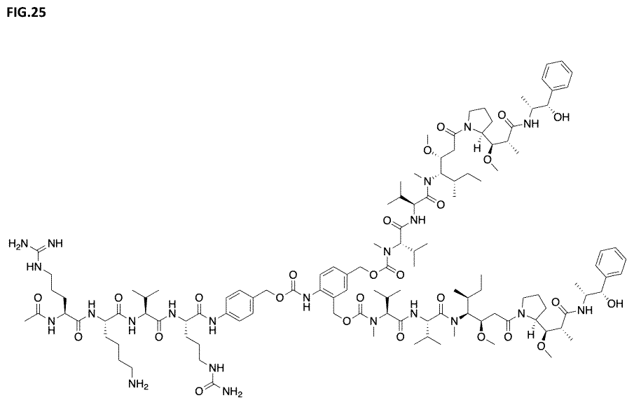

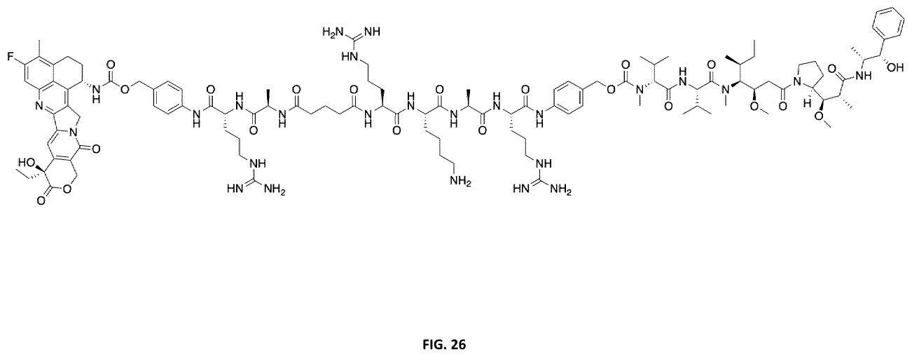

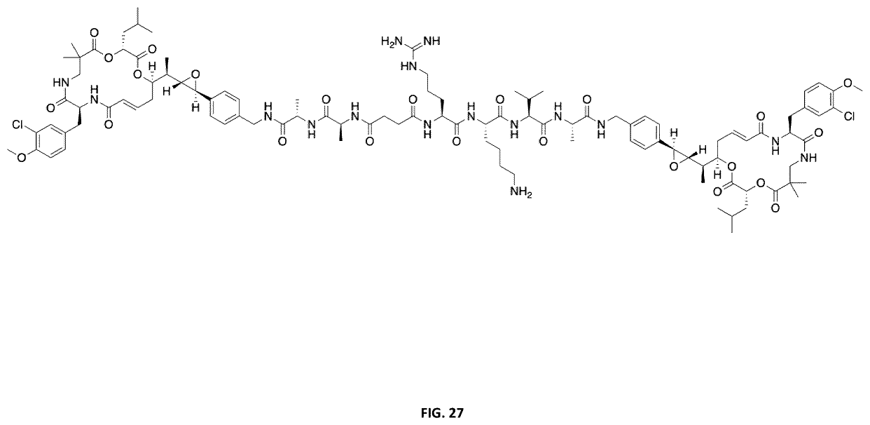

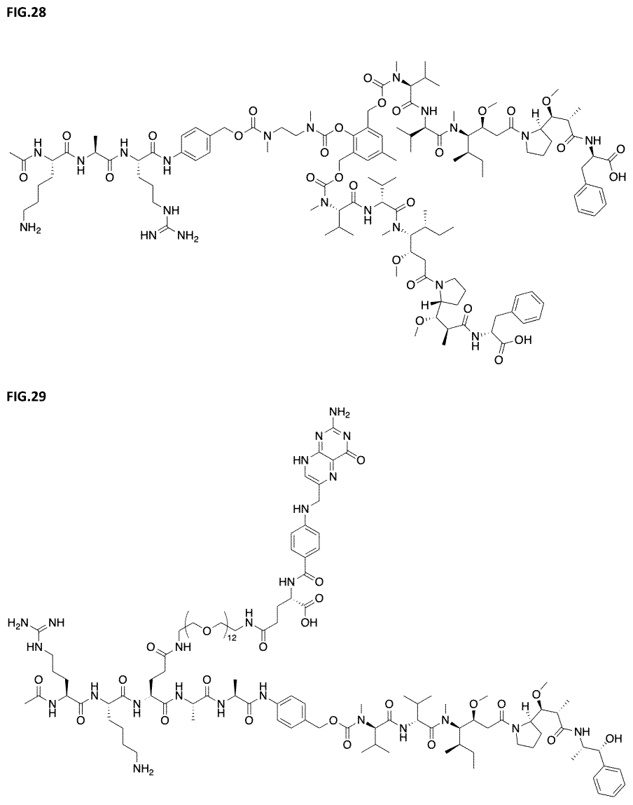

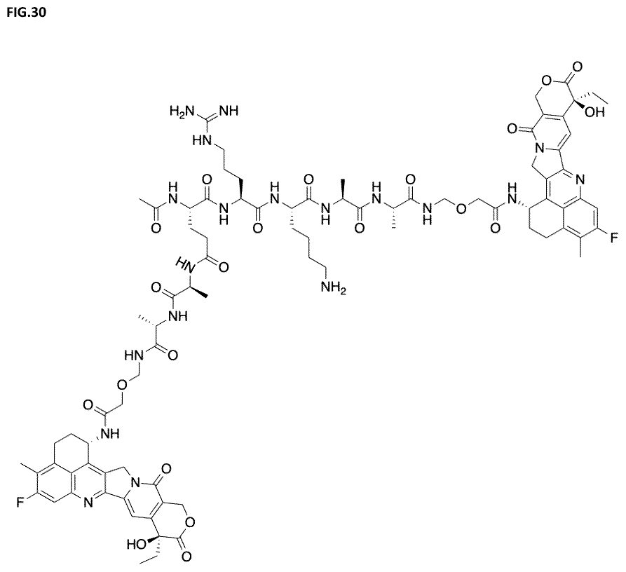

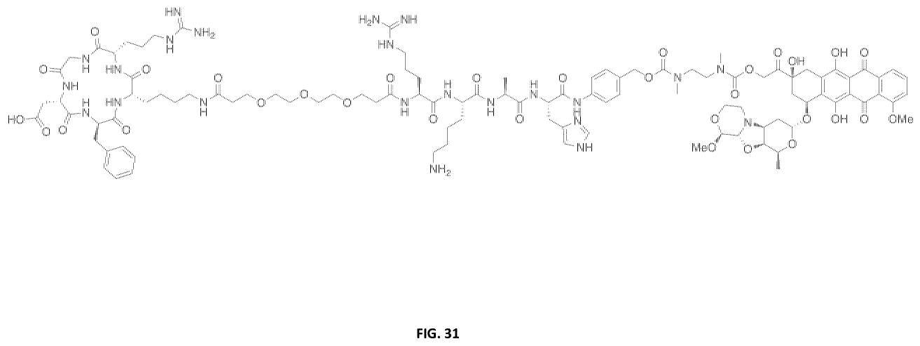

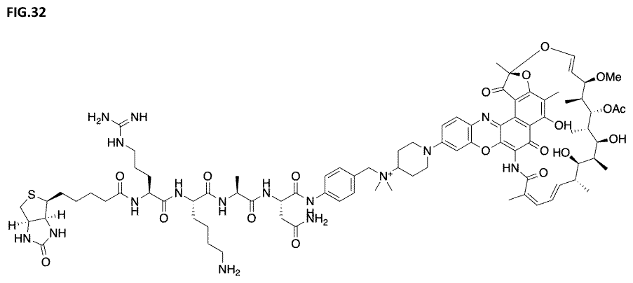

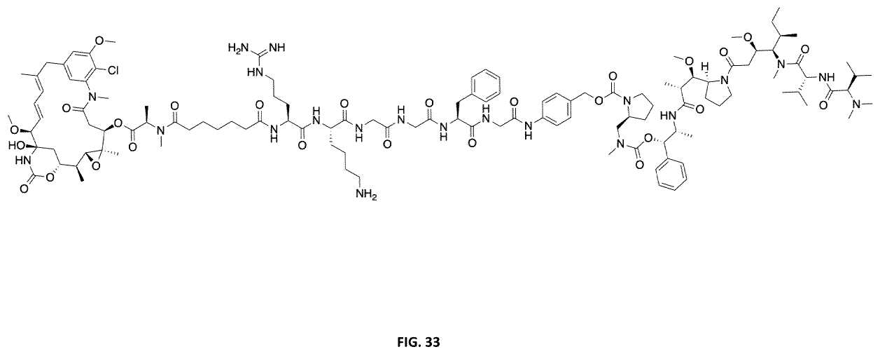

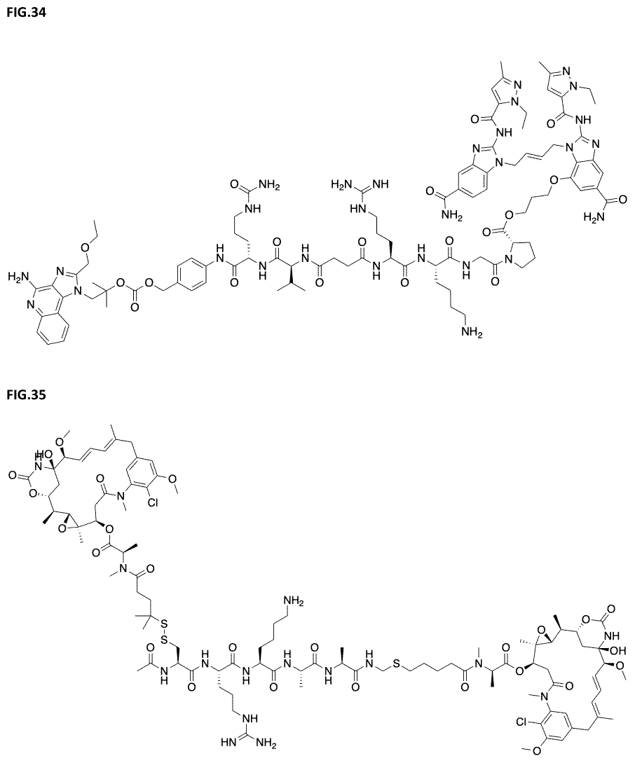

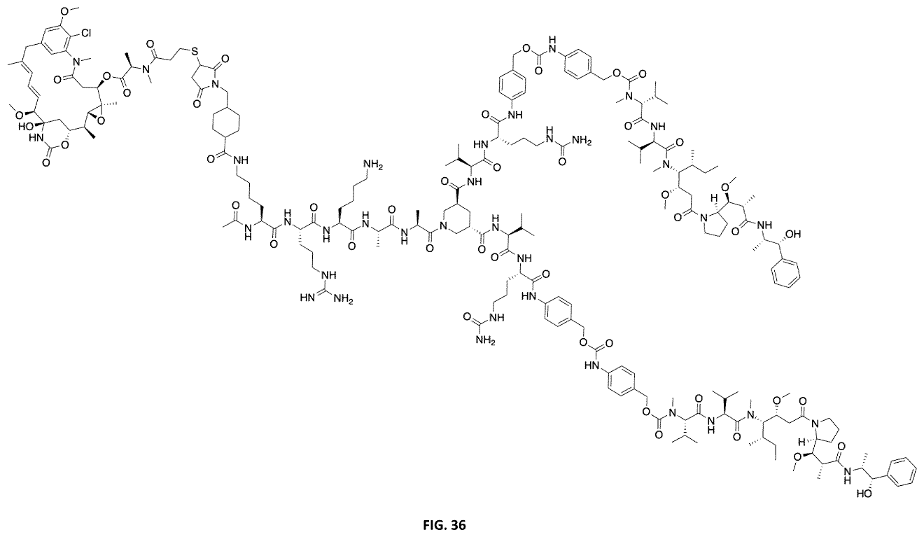

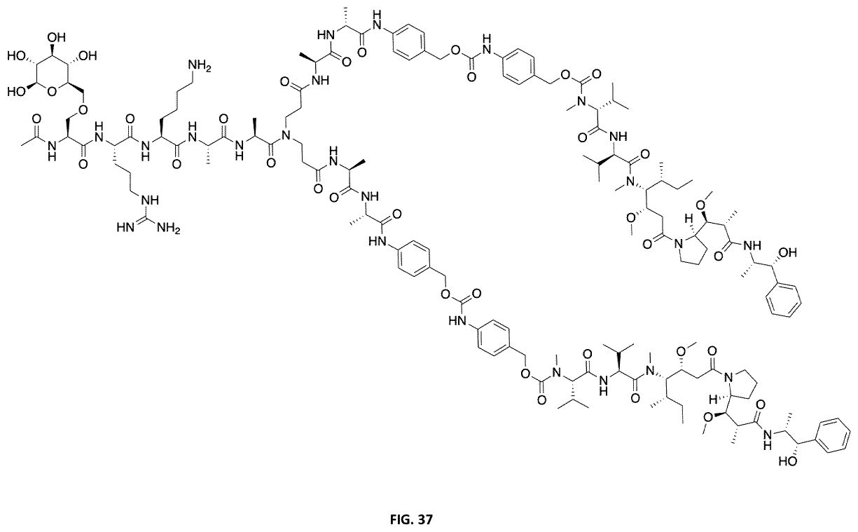

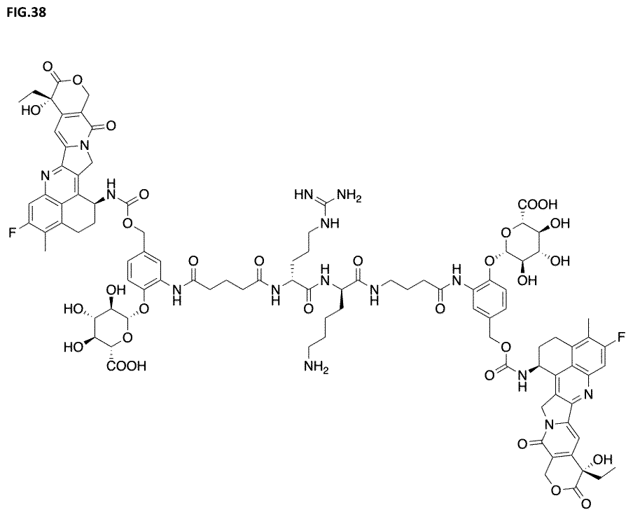

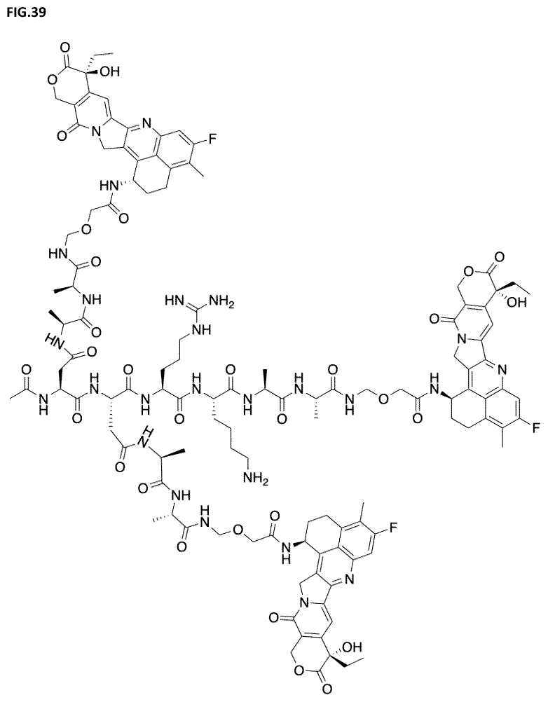

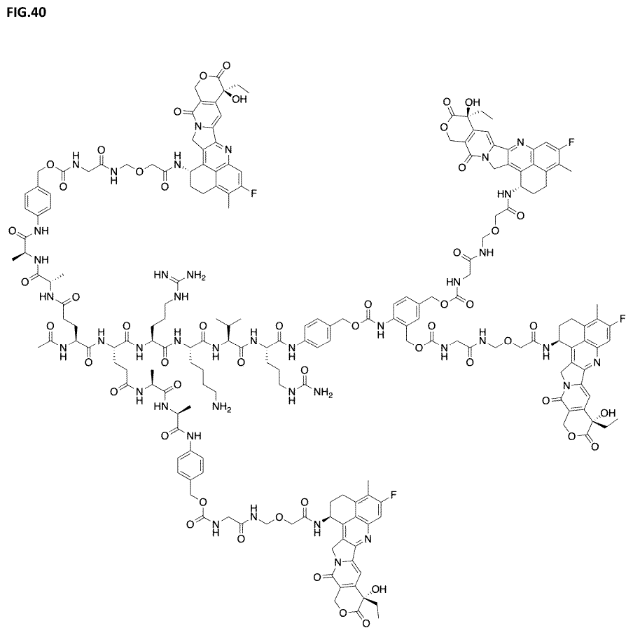

TABLE 8
Conjugation efficiency (%) obtained using MMAE-Maytansine-Cortisol linker-payload to
generate Trastuzumab DAR6 Trastuzumab tri-payload ADC comprising 2 MMAE, 2 Maytansine and 2
Cortisol

| Linker-payload containing tri-payload MMAE-May-CS for | |
|---|---|
| preparation of DAR 6 ADC comprising 2 MMAE, 2 Maytansine | Conjugation efficiency (%) |
| and 2 Cortisol | to Trastuzumab |
| May-RKAE(A-PABC-MMAE)A-Cortisol (SEQ ID NO: 12; FIG. 22) | 99% |

### Example 9: Conjugation of MMAE Linker-Payload Construct Containing a Peptide (According to this Invention) or without a Peptide (NOT According to this Invention) for Preparation of Trastuzumab DAR 4 ADC

### Method

Reaction conditions: 5 mg/ml of native, glycosylated Trastuzumab antibody, MTG at a concentration of 8 U/mg, and 5 molar equivalents of the indicated linker-payload, in BisTris 50 mM pH 7.5 for 22 hours at 37° C. in a rotating thermomixer. Conjugation efficiency was assessed by LCMS as described above.

### Results

A linker according to this invention, comprising a peptide and two MMAE, provided excellent conjugation efficiency (Table 9) to native, fully glycosylated Trastuzumab resulting in a Trastuzumab-DAR 4 MMAE ADC, while in a striking contrast, a structure with an amino-PEG and two MMAE (but without peptide therefore NOT according to this invention) conjugated very poorly to native, fully glycosylated Trastuzumab.

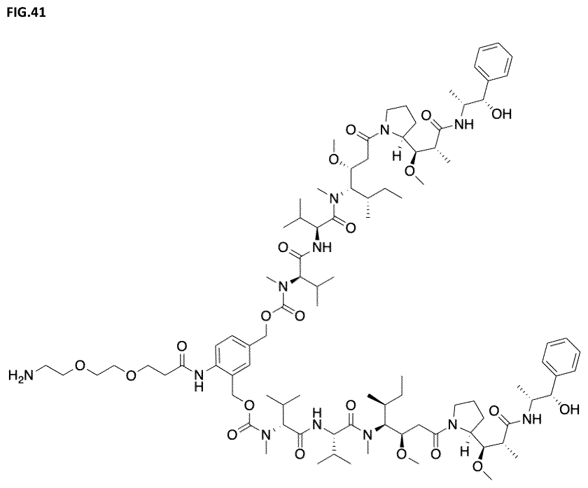

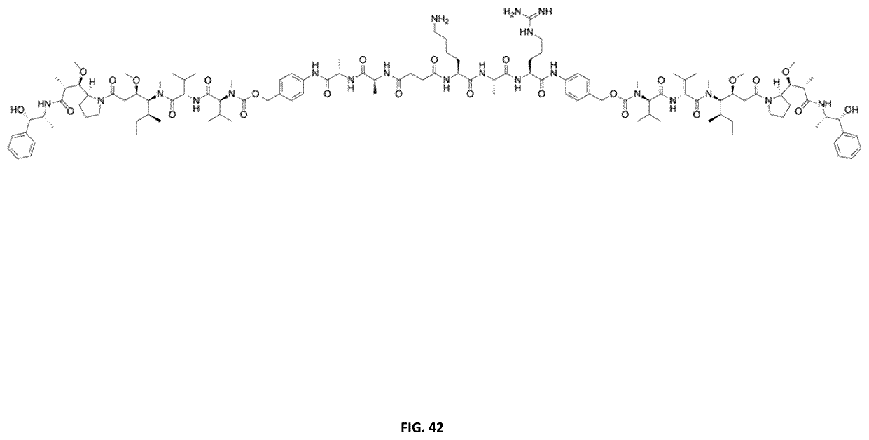

TABLE 6
Conjugation efficiencies (%) obtained using MMAE linker-payload constructs to generate
Trastuzumab DAR 4 ADCs

| Linker-payload with MMAE for preparation of | Conjugation efficiency (%) to |
|---|---|
| DAR 4 ADCs | Trastuzumab |
| RKAA-PABC-(MMAE)₂ (SEQ ID NO: 1; FIG.1) | 95% |
| NH2-PEG2-PABC-(MMAE)₂ (FIG. 41)* | 26% |

*NOT according to this invention

### Example 10: Identification of Optimal Reaction Conditions

To demonstrate the importance of the linker-payload concentration to reach optimal conjugation efficiency, various linker sequences and linker-payload concentrations were tested for conjugation to Trastuzumab.

### Method

As standard conjugation protocol, the following parameters were used: 3.5 mg/ml of native, glycosylated Trastuzumab antibody and MTG at a concentration of 5 U/mg, in Tris 50 mM, pH 7.6, for 24 hours at 37° C. in a rotating thermomixer. The linker-payloads MMAE-PABC-AA-C2-KAR-PABC-MMAE, RKN(PABC-MMAE)A-PABC-MMAE, and RKAA-PABC-(MMAE)₂ were added to the reaction mix at varying concentrations ranging from 2-80 molar equivalents. The parameters are shown in Table 7. Conjugation efficiency was assessed by LCMS as described above.

### Results

Surprisingly, exceptionally high conjugation efficiencies were obtained when 5-20 equivalents of linker-payload were added to the reaction irrespective of the tested linker-payload sequence.

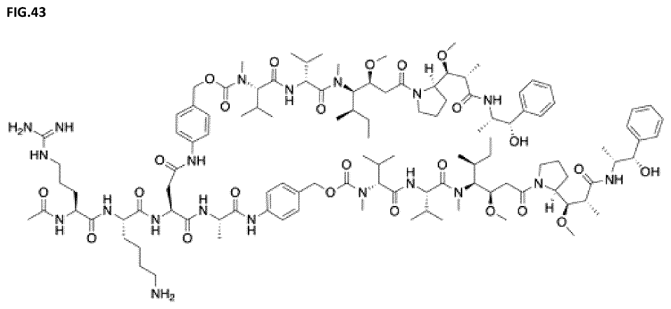

TABLE 7
Conjugation efficiencies of MMAE-PABC-AA-C2-KAR-PABC-MMAE, RKN(PABC-MMAE)A-
PABC-MMAE and RKAA-PABC-(MMAE)₂ to Trastuzumab under different reaction conditions
Effects on reaction parameters on conjugation efficiency

| Parameters | 2 | 5 | 10 | 20 | 40 | 80 |
|---|---|---|---|---|---|---|
| | Molar equivalent of MMAE-PABC-AA-C2-KAR-PABC-MMAE (FIG. 42) vs Trastuzumab | | | | | |
| CE (%) | 54% | 94% | 99% | 96% | 75% | 31% |
| | Molar equivalent of RKN(PABC-MMAE)A-PABC-MMAE (SEQ ID NO: 83; FIG. 43) vs Trastuzumab | | | | | |
| CE (%) | 50% | 82% | 84% | 56% | 19% | 2% |
| | Molar equivalent of RKAA-PABC-(MMAE)₂ (SEQ ID NO: 1; FIG. 1) vs Trastuzumab | | | | | |
| CE (%) | 69% | 97% | 100% | 99% | 91% | 68% |

### Example 11: Anti-Nectin-4 DAR4 ADCs Show Efficient Tumor Growth Inhibition In Vivo in Nectin-4 Positive Solid Tumor Models

The anti-Nectin-4 ADCs according to this invention, ARA-04-MMAE-PABC-AA-C2-RKAA-PABC-MMAE (DAR 4.0) and ARA-04-RKAA-PABC-(MMAE)₂ (DAR 3.8) were investigated in vivo for tumor growth inhibition in a SUM190PT (Nectin-4-positive, solid tumor) xenograft model.

### Method

For SUM190PT xenografts, 2×10⁶ cells were injected into the mammary fat pad of CB17 SCID mice (Janvier). Tumor dimensions and body weights were recorded three times weekly. The tumor volume was calculated according to the formula volume=(width)²×length×0.5. When the average tumor size reached about 200 mm³, mice were allocated using a non-random stratification protocol into the treatment groups comprising six mice each. ADCs were intravenously injected once on the day of randomization. The ADCs according to this invention were produced in-house as described in Example 2. Enfortumab vedotin (DAR 4) was commercially available, bought from a pharmacy. ARA-04-MMAE-PABC-AA-C2-RKAA-PABC-MMAE (DAR 4.0) and ARA-04-RKAA-PABC-(MMAE)₂ (DAR 3.8) were injected at ADC doses corresponding to 10 μg payload dose per kg mouse weight (10 μg/kg). Enfortumab vedotin (DAR 4) was injected 15 μg payload dose per kg mouse weight (15 μg/kg). Mice in the control group were injected with PBS. All mouse experiments were performed in accordance with Swiss guidelines and were approved by the Veterinarian Office of Zurich, Switzerland.

### Results

ADCs according to this invention ARA-04-MMAE-PABC-AA-C2-RKAA-PABC-MMAE and ARA-04-RKAA-PABC-(MMAE)₂ were compared to Enfortumab vedotin in a SUM190PT xenograft model.

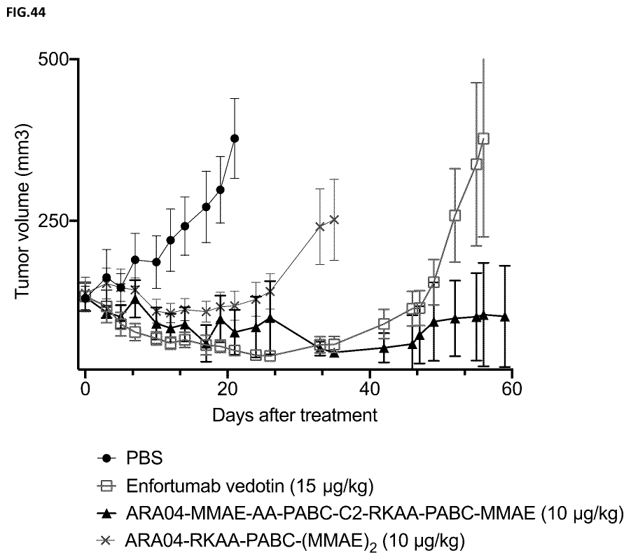

A single i.v. injection of 10 μg/kg weight resulted in efficient anti-tumor responses in all mice for the ADCs according to this invention (FIG. 44). Enfortumab vedotin dosed at 15 μg/kg, showed transient tumor regression with no tumor-free individuals at 20 days after treatment and marked regrowth in 6/6 animals after day 40. Most surprisingly, ARA-04-MMAE-PABC-AA-C2-RKAA-PABC-MMAE showed better efficacy in 4/6 mice, leading to complete long-lasting responses, as opposed to ARA-04-RKAA-PABC-(MMAE)₂ injected at the same dose which resulted in tumor control for approximately 20 days in 6/6 mice followed by re-growth of tumors. Therefore, and very surprisingly, these data show a more efficient anti-tumor activity of ARA-04-MMAE-PABC-AA-C2-RKAA-PABC-MMAE, where payloads are coupled to each N- and C-terminal end of a peptide linker, compared to ARA-04-RKAA-PABC-(MMAE)₂, where the payloads are only coupled to the C-terminal end of a peptide linker.

It is summarized that anti-Nectin-4 ADCs, ARA-04-MMAE-PABC-AA-C2-RKAA-PABC-MMAE and ARA-04-RKAA-PABC-(MMAE)₂, according to this invention, consisting of the same antibody and payload as their respective benchmark ADC (Enfortumab vedotin) are active in vivo. Surprisingly, only ARA-04-MMAE-PABC-AA-C2-RKAA-PABC-MMAE, where the payloads are coupled to each N- and C-terminal end of a peptide linker showed superior efficacy providing survival advantage.

### Example 12: Anti-CD79b DAR4 ADCs Show Efficient Tumor Growth Inhibition In Vivo in CD79b-Positive Liquid Tumor Models

The anti-CD79b ADCs according to this invention, ARA-01-MMAE-PABC-AA-C2-RKAA-PABC-MMAE (DAR 4.0) and ARA-01-RKAA-PABC-(MMAE)₂ (DAR 3.9) were investigated in vivo tumor growth inhibition in a Ramos (CD79b-positive, liquid tumor) xenograft model.

### Method

For Ramos xenografts, 20×10⁶ cells were injected subcutaneously into CB17 SCID mice (Janvier). Tumor dimensions and body weights were recorded three times weekly. The tumor volume was calculated according to the formula volume=(width)²×length×0.5. When the average tumor size reached about 200 mm³, mice were allocated using a non-random stratification protocol into the treatment groups comprising six mice each. ADCs were intravenously injected once on the day of randomization. The ADCs according to this invention were produced in-house as described in Example 2. ARA-01-MMAE-PABC-AA-C2-RKAA-PABC-MMAE (DAR 4.0) and ARA-01-RKAA-PABC-(MMAE)₂ (DAR 3.9) were once intravenously injected at ADC doses corresponding to 25 μg payload dose per kg mouse weight (25 μg/kg). Mice in the control group were injected with PBS. All mouse experiments were performed in accordance with Swiss guidelines and were approved by the Veterinarian Office of Zurich, Switzerland.

### Results

ADCs according to this invention ARA-01-MMAE-PABC-AA-C2-RKAA-PABC-MMAE and ARA-01-RKAA-PABC-(MMAE)₂ were compared to each other in a Ramos xenograft model.

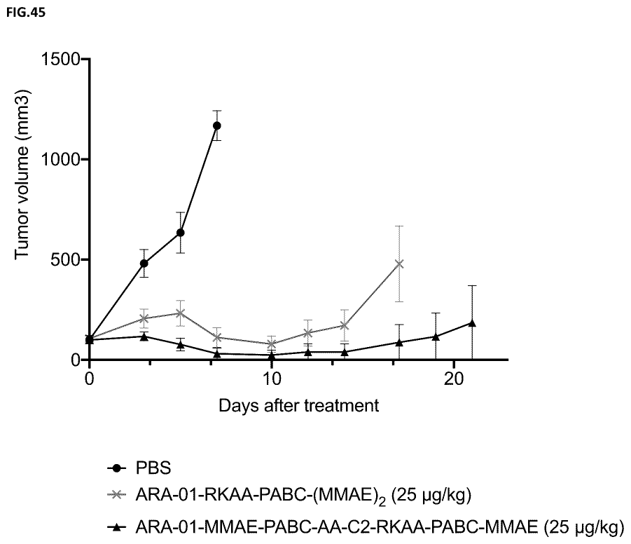

A single i.v. injection of 25 μg/kg resulted in efficient anti-tumor responses in all mice for the ADCs according to this invention (FIG. 45) compared to PBS-treated control group. Surprisingly, ARA-01-MMAE-PABC-AA-C2-RKAA-PABC-MMAE showed superior efficacy, with complete tumor elimination in 5/6 mice at day 21 after treatment, compared to ARA-01-RKAA-PABC-(MMAE)₂ injected at the same dose, which resulted in a transient decrease of the tumors volumes, followed by tumor re-growth in all animals. These data corroborate the results from Example 11 and substantiate the finding that ADCs where the payloads are coupled to each N- and C-terminal end of a peptide linker surprisingly show more efficient anti-tumor activity than the comparator ADCs where the payloads are only coupled to the C-terminal end.

It is summarized that anti-CD79b ADCs, ARA-01-MMAE-PABC-AA-C2-RKAA-PABC-MMAE and ARA-01-RKAA-PABC-(MMAE)₂, according to this invention, are active in vivo. Surprisingly, ARA-01-MMAE-PABC-AA-C2-RKAA-PABC-MMAE, where the payloads are coupled to each N- and C-terminal end of a peptide linker, showed superior efficacy providing survival advantage compared to ARA-01-RKAA-PABC-(MMAE)₂, where the payloads are only coupled to the C-terminal end of a peptide linker, corroborating data shown in FIG. 11 where it is shown that MMAE-PABC-AA-C2-RKAA-PABC-MMAE linker resulted in superior anti-tumor efficacy as compared to RKAA-PABC-(MMAE)₂.

1. A peptide linker comprising
a) an amino acid residue comprising a primary amine; and
b) two or more payloads;
wherein each of the two or more payloads can be independently attached to:
i) an N-terminal end of the peptide linker,
ii) a C-terminal end of the peptide linker, or
iii) a side chain of an amino acid residue comprised in the peptide linker.

2. The peptide linker according to claim 1, wherein the primary amine comprised in the amino acid residue is
a) a primary amine in a side chain of a lysine, a lysine derivative or a lysine mimetic; or
b) a primary amine comprised in an N-terminal amino acid residue having the structure NH₂—(Y)—COOH.

3. The peptide linker according to claim 2, wherein Y is (R₂C)ₙ and wherein n is an integer ranging from 1 to 20, from 1 to 15, from 1 to 10.

4. The peptide linker according to claim 3, wherein at least one R moiety of each —(R₂C)— monomer is hydrogen or wherein both R moieties of each —(R₂C)— monomer are hydrogen.

5. The peptide linker according to claim 1, wherein the linker comprises not more than 25 amino acid residues.

6. The peptide linker according to claim 1, wherein the linker comprises at least one arginine and/or histidine residue.

7. The peptide linker according to claim 1, wherein the linker comprises the sequence motif RK.

8. The peptide linker according to claim 1, wherein the linker comprises any one of the amino acid sequences set forth in SEQ ID NO:1-29 or 82-93.

9. The peptide linker according to claim 1, wherein the linker comprises between 2 and 4 payloads.

10. The peptide linker according to claim 1, wherein the linker consists of or comprises the following structure (in N->C direction):
[payload1]-[(Aa)ₘ-(Lys)-(Aa)ₙ-(Arg/His)-(Aa)ₒ]-[payload2]; wherein
[payload 1] and [payload 2] are payloads,
(Aa) may be any amino acid residue;
m, n and o may be integers ranging from 0 to 10;
(Arg) may be an arginine residue, an arginine mimetic or an arginine derivative;
(His) may be a histidine residue, a histidine mimetic or a histidine derivative
(Lys) is a lysine residue, a lysine mimetic or a lysine derivative,
wherein [payload 1] is directly or indirectly attached to an N-terminal end of an (Aa) or (Lys) residue, and
wherein [payload 2] is directly or indirectly attached to a C-terminal end of an (Aa) or (Arg/His) residue.

11-19. (canceled)

20. The peptide linker according claim 1, wherein at least one payload is attached to a side chain of an amino acid residue comprised in the peptide linker.

21. (canceled)

22. The peptide linker according to claim 1, wherein the peptide linker comprises two peptide moieties, and wherein the two peptide moieties are connected via their N-terminal amino acid residues with a dicarboxylic acid linker, or an activated version thereof.

23. The peptide linker according to claim 22, wherein the linker comprises the structure:
[payload1]-[peptide1]-[dicarboxylic acid]-[peptide2]-[payload2]; wherein
[payload 1] and [payload 2] are payloads,
[peptide 1] is a first peptide moiety,
[peptide 2] is a second peptide moiety, and
[dicarboxylic acid] is a dicarboxylic acid;
wherein at least one of [peptide 1] and/or [peptide 2] comprises a free amine,
wherein the N-terminal end of [peptide 1] and the N-terminal end of [peptide 2] are connected via the dicarboxylic acid,
wherein [payload 1] is attached to the C-terminal end of [peptide 1], and
wherein [payload 2] is attached to the C-terminal end of [peptide 2] linker.

24-28. (canceled)

29. An antibody-payload conjugate comprising an antibody conjugated to a peptide linker comprising
a) an amino acid residue comprising a primary amine; and
b) two or more payloads;
wherein each of the two or more payloads can be independently attached to:
i) an N-terminal end of the peptide linker,
ii) a C-terminal end of the peptide linker, or
iii) a side chain of an amino acid residue comprised in the peptide linker.

30. The antibody-payload conjugate according to claim 29, wherein the peptide linker is conjugated to the antibody via an isopeptide bond formed between a γ-carboxamide group of a glutamine residue comprised in the antibody and the primary amine comprised in an amino acid residue of the peptide linker.

31. (canceled)

32. The antibody-payload conjugate according to claim 29, wherein the peptide linker is conjugated to a glutamine residue comprised in an Fc domain of the antibody.

33. The antibody-payload conjugate according to claim 32, wherein the glutamine residue to which the peptide linker is conjugated is glutamine residue Q295 (EU numbering) of the C_{H}2 domain of an IgG antibody.

34. (canceled)

35. The antibody-payload conjugate according to claim 32, wherein the glutamine residue to which the peptide linker is conjugated is glutamine residue N297Q (EU numbering) of the CH2 domain of an aglycosylated IgG antibody.

36-37. (canceled)

38. The antibody-payload conjugate according to claim 29, wherein the antibody is a glycosylated IgG antibody.

39. The antibody-payload conjugate according to claim 38, wherein the IgG antibody is glycosylated at residue N297 (EU numbering) of the CH2 domain.

40-42. (canceled)

43. A method for the preparation of an antibody-payload conjugate comprising a step of conjugating a peptide linker to an antibody, wherein the peptide linker comprises
a) an amino acid residue comprising a primary amine; and
b) two or more payloads;
wherein each of the two or more payloads can be independently attached to:
i) an N-terminal end of the peptide linker,
ii) a C-terminal end of the peptide linker, or
iii) a side chain of an amino acid residue comprised in the peptide linker.

44. A method for the conjugation of a peptide linker comprising two or more payloads to an antibody using a transglutaminase (TG), the method comprising a) mixing the antibody, the peptide linker and the TG within a fluid, thereby conjugating the linker-payload to the antibody in one step under the catalyzing effect of the TG, and b) extracting the conjugate obtained in step a) from the fluid.

45-69. (canceled)

70. A pharmaceutical composition comprising the antibody-payload conjugate according to claim 29 and at least one pharmaceutically acceptable ingredient.

71.-81. (canceled)

82. A method of treating or preventing a neoplastic disease, said method comprising administering to a patient in need thereof an antibody-payload conjugate comprising an antibody conjugated to a peptide linker comprising
a) an amino acid residue comprising a primary amine; and
b) two or more payloads;
wherein each of the two or more payloads can be independently attached to:
i) an N-terminal end of the peptide linker,
ii) a C-terminal end of the peptide linker, or
iii) a side chain of an amino acid residue comprised in the peptide linker.


SEQUENCE LISTING (93 sequences)

SEQ ID NO: 1 - protein, 4 aa, synthetic construct
  REGION 1..4: note = Linker Sequence 1
RKAA

SEQ ID NO: 2 - skipped

SEQ ID NO: 3 - protein, 5 aa, synthetic construct
  REGION 1..5: note = Linker Sequence 3
RKARA

SEQ ID NO: 4 - protein, 6 aa, synthetic construct
  REGION 1..6: note = Linker Sequence 4
RKAAAA

SEQ ID NO: 5 - protein, 8 aa, synthetic construct
  REGION 1..8: note = Linker Sequence 5
RKAAAAAA

SEQ ID NO: 6 - protein, 8 aa, synthetic construct
  REGION 1..8: note = Linker Sequence 6
RKAASGSG

SEQ ID NO: 7 - protein, 4 aa, synthetic construct
  REGION 1..4: note = Linker Sequence 7
RKHA

SEQ ID NO: 8 - protein, 6 aa, synthetic construct
  REGION 1..6: note = Linker Sequence 8
RKHAAA

SEQ ID NO: 9 - skipped

SEQ ID NO: 10 - protein, 4 aa, synthetic construct
  REGION 1..4: note = Linker Sequence 10
GGRG

SEQ ID NO: 11 - protein, 6 aa, synthetic construct
  REGION 1..6: note = Linker Sequence 11
EARKAA

SEQ ID NO: 12 - protein, 5 aa, synthetic construct
  REGION 1..5: note = Linker Sequence 12
RKAEA

SEQ ID NO: 13-16 - skipped (4 entries)

SEQ ID NO: 17 - protein, 4 aa, synthetic construct
  REGION 1..4: note = Linker Sequence 17
RKAR

SEQ ID NO: 18 - protein, 4 aa, synthetic construct
  REGION 1..4: note = Linker Sequence 18
RKVA

SEQ ID NO: 19 - skipped

SEQ ID NO: 20 - protein, 5 aa, synthetic construct
  REGION 1..5: note = Linker Sequence 20
RKEAA

SEQ ID NO: 21 - protein, 5 aa, synthetic construct
  REGION 1..5: note = Linker Sequence 21
ERKAA

SEQ ID NO: 22 - protein, 4 aa, synthetic construct
  REGION 1..4: note = Linker Sequence 22
RKAH

SEQ ID NO: 23 - protein, 4 aa, synthetic construct
  REGION 1..4: note = Linker Sequence 23
RKAN

SEQ ID NO: 24 - protein, 6 aa, synthetic construct
  REGION 1..6: note = Linker Sequence 24
RKGGFG

SEQ ID NO: 25 - protein, 4 aa, synthetic construct
  REGION 1..4: note = Linker Sequence 25
RKGP

SEQ ID NO: 26 - protein, 5 aa, synthetic construct
  REGION 1..5: note = Linker Sequence 26
KRKAA

SEQ ID NO: 27 - protein, 5 aa, synthetic construct
  REGION 1..5: note = Linker Sequence 27
SRKAA

SEQ ID NO: 28 - protein, 6 aa, synthetic construct
  REGION 1..6: note = Linker Sequence 28
DDRKAA

SEQ ID NO: 29 - protein, 6 aa, synthetic construct
  REGION 1..6: note = Linker Sequence 29
  NON_STD 6: note = X is Citrulline
EERKVX

SEQ ID NO: 30 - protein, 4 aa, synthetic construct
  REGION 1..4: note = cleavable peptide 1
GFLG

SEQ ID NO: 31 - protein, 4 aa, synthetic construct
  REGION 1..4: note = Cleavable peptide 2
ALAL

SEQ ID NO: 32 - protein, 4 aa, synthetic construct
  REGION 1..4: note = Cleavable peptide 3
GGFG

SEQ ID NO: 33 - protein, 4 aa, synthetic construct
  REGION 1..4: note = N-terminal linker 1
AAAN

SEQ ID NO: 34-36 - skipped (3 entries)

SEQ ID NO: 37 - protein, 4 aa, synthetic construct
  REGION 1..4: note = Q-Tag 1
LLQG

SEQ ID NO: 38 - protein, 6 aa, synthetic construct
  REGION 1..6: note = Q-Tag 2
LSLSQG

SEQ ID NO: 39 - protein, 8 aa, synthetic construct
  REGION 1..8: note = Q-tag 3
GGGLLQGG

SEQ ID NO: 40 - protein, 5 aa, synthetic construct
  REGION 1..5: note = Q-Tag 4
GLLQG

SEQ ID NO: 41 - skipped

SEQ ID NO: 42 - protein, 10 aa, synthetic construct
  REGION 1..10: note = Q-Tag 6
GSPLAQSHGG

SEQ ID NO: 43 - protein, 7 aa, synthetic construct
  REGION 1..7: note = Q-Tag 7
GLLQGGG

SEQ ID NO: 44 - protein, 6 aa, synthetic construct
  REGION 1..6: note = Q-Tag 8
GLLQGG

SEQ ID NO: 45 - protein, 4 aa, synthetic construct
  REGION 1..4: note = Q-Tag 9
GLLQ

SEQ ID NO: 46 - protein, 8 aa, synthetic construct
  REGION 1..8: note = Q-Tag 10
LLQLLQGA

SEQ ID NO: 47 - protein, 5 aa, synthetic construct
  REGION 1..5: note = Q-Tag 11
LLQGA

SEQ ID NO: 48 - protein, 7 aa, synthetic construct
  REGION 1..7: note = Q-Tag 12
LLQYQGA

SEQ ID NO: 49 - protein, 6 aa, synthetic construct
  REGION 1..6: note = Q-Tag 13
LLQGSG

SEQ ID NO: 50 - protein, 6 aa, synthetic construct
  REGION 1..6: note = Q-Tag 14
LLQYQG

SEQ ID NO: 51 - protein, 7 aa, synthetic construct
  REGION 1..7: note = Q-Tag 15
LLQLLQG

SEQ ID NO: 52 - protein, 5 aa, synthetic construct
  REGION 1..5: note = Q-Tag 16
SLLQG

SEQ ID NO: 53 - protein, 5 aa, synthetic construct
  REGION 1..5: note = Q-Tag 17
LLQLQ

SEQ ID NO: 54 - protein, 6 aa, synthetic construct
  REGION 1..6: note = Q-Tag 18
LLQLLQ

SEQ ID NO: 55 - protein, 5 aa, synthetic construct
  REGION 1..5: note = Q-Tag 19
LLQGR

SEQ ID NO: 56 - protein, 8 aa, synthetic construct
  REGION 1..8: note = Q-Tag 20
EEQYASTY

SEQ ID NO: 57 - protein, 8 aa, synthetic construct
  REGION 1..8: note = Q-Tag 21
EEQYQSTY

SEQ ID NO: 58 - protein, 8 aa, synthetic construct
  REGION 1..8: note = Q-Tag 22
EEQYNSTY

SEQ ID NO: 59 - protein, 6 aa, synthetic construct
  REGION 1..6: note = Q-Tag 23
EEQYQS

SEQ ID NO: 60 - protein, 7 aa, synthetic construct
  REGION 1..7: note = Q-Tag 24
EEQYQST

SEQ ID NO: 61 - protein, 7 aa, synthetic construct
  REGION 1..7: note = Q-Tag 25
EQYQSTY

SEQ ID NO: 62 - protein, 4 aa, synthetic construct
  REGION 1..4: note = Q-Tag 26
QYQS

SEQ ID NO: 63 - protein, 6 aa, synthetic construct
  REGION 1..6: note = Q-Tag 27
QYQSTY

SEQ ID NO: 64 - protein, 5 aa, synthetic construct
  REGION 1..5: note = Q-Tag 28
YRYRQ

SEQ ID NO: 65 - protein, 5 aa, synthetic construct
  REGION 1..5: note = Q-Tag 29
DYALQ

SEQ ID NO: 66 - protein, 7 aa, synthetic construct
  REGION 1..7: note = Q-Tag 30
FGLQRPY

SEQ ID NO: 67 - protein, 10 aa, synthetic construct
  REGION 1..10: note = Q-Tag 31
EQKLISEEDL

SEQ ID NO: 68-69 - skipped (2 entries)

SEQ ID NO: 70 - protein, 5 aa, synthetic construct
  REGION 1..5: note = Q-Tag 34
LLQGG

SEQ ID NO: 71 - protein, 446 aa, synthetic construct
  REGION 1..446: note = Polatuzumab heavy chain
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 218 aa, synthetic construct
  REGION 1..218: note = Polatuzumab light chain
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 450 aa, synthetic construct
  REGION 1..450: note = Trastuzumab heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 214 aa, synthetic construct
  REGION 1..214: note = Trastuzumab light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 447 aa, synthetic construct
  REGION 1..447: note = Enfortumab heavy chain
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 214 aa, synthetic construct
  REGION 1..214: note = Enfortumab light chain 1
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 214 aa, synthetic construct
  REGION 1..214: note = Enfortumab light chain 2
DIQMTQSPSSVSASVGDRVTITCRASQGISGWLAWYQQKPGKAPKFLIYAASTLESGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 335 aa, synthetic construct
  REGION 1..335: note = S. mobarensis MTG 1
FRAPDSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 79 - protein, 407 aa, synthetic construct [sequence omitted]

SEQ ID NO: 80 - protein, 395 aa, synthetic construct
  REGION 1..395: note = S. mobarensis MTG 3
MHRRIHAVGQARPPPTMARGKETKSYAETYRLTADDVANINALNESAPAASSAGPSFRAPDSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRSAPGTGLVDMSRDRNIPRSPTSPGEGFVNFDYGWFGAQTEADADKTVWTHGNHYHAPNGSLGCHACLTRASSATGSEGYSDFDRGEPYVVSPSPSPRMLEHRPRQGKAGLA

SEQ ID NO: 81 - protein, 395 aa, synthetic construct; identical to SEQ ID NO: 80
  REGION 1..395: note = Streptoverticillium ladakanum MTG

SEQ ID NO: 82 - protein, 4 aa, synthetic construct
  REGION 1..4: note = Linker sequence 30
RKDA

SEQ ID NO: 83 - protein, 4 aa, synthetic construct
  REGION 1..4: note = Linker sequence 31
RKNA

SEQ ID NO: 84 - protein, 6 aa, synthetic construct
  REGION 1..6: note = Linker Sequence 32
QARKAA

SEQ ID NO: 85 - protein, 5 aa, synthetic construct
  REGION 1..5: note = Linker sequence 33
RKAQA

SEQ ID NO: 86 - protein, 5 aa, synthetic construct
  REGION 1..5: note = Linker sequence 34
RKQAA

SEQ ID NO: 87 - protein, 5 aa, synthetic construct
  REGION 1..5: note = Linker sequence 35
QRKAA

SEQ ID NO: 88 - protein, 6 aa, synthetic construct
  REGION 1..6: note = Linker sequence 36
DNRKAA

SEQ ID NO: 89 - protein, 6 aa, synthetic construct
  REGION 1..6: note = Linker sequence 37
NDRKAA

SEQ ID NO: 90 - protein, 6 aa, synthetic construct
  REGION 1..6: note = Linker sequence 38
NNRKAA

SEQ ID NO: 91 - protein, 6 aa, synthetic construct
  REGION 1..6: note = Linker sequence 39
  MOD_RES 6: note = X is Citrulline
EQRKVX

SEQ ID NO: 92 - protein, 6 aa, synthetic construct
  REGION 1..6: note = Linker sequence 40
  MOD_RES 6: note = X is Citrulline
QERKVX

SEQ ID NO: 93 - protein, 6 aa, synthetic construct
  REGION 1..6: note = Linker sequence 41
  MOD_RES 6: note = X is Citrulline
QQRKVX